(19) United States
(12) Patent Application Publication
(10) Pub. No.: US 2012/0220560 A1
(43) Pub. Date: Aug. 30, 2012

(54) STEROID TETROL SOLID STATE FORMS

(76) Inventors: Steven K. White, San Diego, CA (US); Erin E. Jansen, San Diego, CA (US)

(21) Appl. No.: 13/328,374

(22) Filed: Dec. 16, 2011

Related U.S. Application Data
(60) Provisional application No. 61/424,156, filed on Dec. 17, 2010.

(51) Int. Cl. A61K 31/565; A61P 29/00; A61P 3/08; A61P 3/00; A61P 3/10; A61P 3/04; C07J 1/00; A61P 37/00

(52) U.S. Cl. 514/182; 552/615

(57) ABSTRACT

The invention relates to solid state forms of androst-5-ene-3β,7β,16α,17β-tetrol, formulations containing or prepared from such solid state forms and use of these materials for modulating unwanted inflammation including acute and chronic non-productive inflammation. The formulations can be used to prevent, treat or slow the progression of conditions related to autoimmunity and metabolic disorders such as arthritis, multiple sclerosis, ulcerative colitis, Type 1 diabetes and Type 2 diabetes.

### CROSS REFERENCE TO RELATED APPLICATIONS

This nonprovisional U.S. patent application claims priority under 35 USC §119(e) from pending U.S. provisional application No. 61/424,156, filed on Dec. 17, 2010, which is incorporated herein by reference in its entirety.

### FIELD OF THE INVENTION

The field of the invention relates to solid state forms, including crystalline forms, of androst-5-ene-3β,7β,16α,17β-tetrol. The invention further relates to solid formulations comprising one or more crystalline forms of androst-5-ene-3β,7β,16α,17β-tetrol and to methods for using the crystalline forms in preparing solid and liquid formulations and uses of these formulations for the treatment of inflammation-based or inflammation-driven diseases or conditions including autoimmune diseases, lung inflammation conditions and inflammatory bowel diseases. Other conditions that can be treated with formulations comprised or prepared from crystalline forms of androst-5-ene-3β,7β,16α,17β-tetrol include metabolic and cardiovascular conditions, neurodegenerative diseases and hyperproliferation conditions such as cancer. Unit dosage forms for the solid and liquid formulations are also included.

### BACKGROUND OF THE INVENTION

The ability of a substance to exist in more than one crystalline form is generally referred to as polymorphism and these different crystalline forms are typically named “polymorphs” and may be referred to by certain analytical properties such their X-ray powder diffraction (XRPD) patterns. In general, polymorphism reflects the ability of a molecule to change its conformation or to form different intermolecular and intramolecular interactions. This can result in different atom arrangements that are reflected in the crystal lattices of different polymorphs. However, polymorphism is not a universal feature of solids, since some molecules can exist in one or more crystal forms while other molecules do not. Therefore, the existence or extent of polymorphism for a given compound is unpredictable.

The different polymorphs of a substance posses different crystal lattice energies and thus each such crystalline form typically shows one or more different physical properties in the solid state, such as density, melting point, color, stability, dissolution rate, flowability, compatibility with milling, granulation and compacting and/or uniformity of distribution [See, e.g., P. DiMartino, et al., J. Thermal Anal. 48:447-458 (1997)]. The capacity of any given compound to occur in one or more crystalline forms is unpredictable as are the physical properties of any single crystalline form. The physical properties of a polymorphic form may affect its suitability in pharmaceutical formulations. For example, those properties can affect positively or negatively the stability, dissolution and bioavailability of a solid-state formulation, which subsequently affects suitability or efficacy of such formulations in treating disease.

An individual crystalline form (i.e., a polymorphic form) having one or more desirable properties can be suitable for the development of a pharmaceutical formulation having desired property(ies). Existence of a compound with another specific crystalline form(s) that has an undesirable property(ies) can impede or prevent development of a desired polymorphic form of the compound as a pharmaceutical agent.

In the case of a chemical substance that exists in more than one polymorphic form, the less thermodynamically stable forms can occasionally convert to the more thermodynamically stable form at a given temperature after a sufficient period of time. When this transformation is rapid, such a thermodynamically unstable form is referred to as a “metastable” form. In some instances, such as in a suitable formulation, a metastable form may exhibit sufficient chemical and physical stability under normal storage conditions to permit its use in a commercial form.

### SUMMARY OF THE INVENTION

In one principal embodiment the invention provides new crystalline forms of 10R,13S-dimethyl 2,3,4,7,8R,9S,10,11,12,13,14S,15,16,17-hexadecahydro-1H-cyclopenta[a]phenanthrene-3R,7R,16R,17S-tetrol, which is represented by Formula 1B.

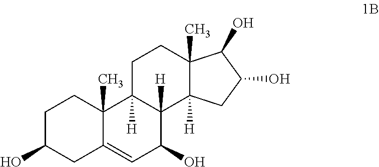

The Formula 1B compound (hereafter also referred to as androst-5-ene-3β,7β,16α,17β-tetrol or 3β-tetrol) has been prepared in various solid state forms and in particular crystalline forms referred herein as Form Iβ, Form IIβ, Form IIIβ, Form IVβ, Form Vβ, Form VIβ, Form VIIβ, Form VIIIβ, Form IXβ and Form Xβ. Solid state forms of 3β-tetrol are suitable for treating acute or chronic conditions related to or associated with unwanted inflammation, including lung inflammation conditions, bowel inflammation conditions, liver inflammation conditions, metabolic and cardiovascular conditions, autoimmune conditions, hyperproliferation conditions, neurodegenerative conditions, ischemia-reperfusion injury and related conditions.

Formulations comprising or prepared from a solid state form of 3β-tetrol, wherein the solid state from is a crystalline form, include Form Iβ substantially free or essentially free of one or more solid state forms optionally selected from the group consisting of Form IIβ, Form IIIβ, Form IVβ, Form Vβ, Form VIβ, Form VIIβ, Form VIIIβ, Form IXβ and Form Xβ.

Other formulations comprising or prepared from a solid state form of 3β-tetrol, wherein the solid state from is a crystalline form include Form IIβ, Form IIIβ, Form IVβ, Form Vβ, Form VIβ, Form VIIβ, Form VIIIβ, Form IXβ and Form Xβ substantially free or essentially free of Form Iβ or a mixture of two or more crystalline forms, preferably two, selected from the group consisting of Form IIβ, Form IIIβ, Form IVβ, Form Vβ, Form VIβ or from the group consisting of Form VIIβ, Form VIIIβ, Form IXβ and Form Xβ wherein Form Iβ is absent or is a minor component relative to the total crystalline content of 3β-tetrol.

A preferred crystalline form of 3β-tetrol is Form IVβ, substantially free or essentially free of one or more solid state forms optionally selected from the group consisting of Form Iβ, Form IIβ, Form IIIβ, Form Vβ and Form VIβ.

Another preferred crystalline form of 3β-tetrol is Form VIIβ, substantially free or essentially free of one or more solid state forms optionally selected from the group consisting of Form Iβ, Form IIβ, Form IIIβ, Form Vβ and Form VIβ or optionally selected from the group consisting of Form VIIIβ, Form Iβ and Form Xβ.

Yet another preferred crystalline form of 3β-tetrol is Form VIIIβ, substantially free or essentially free of one or more solid state forms optionally selected from the group consisting of Form Iβ, Form IIβ, Form IIIβ, Form Vβ and Form VIβ or optionally selected from the group consisting of Form VIIβ, Form IXβ and Form Xβ.

Conditions related to metabolic conditions that can be treated with the 3β-tetrol solid state forms disclosed herein include hyperglycemia, insulin resistance, Type 2 diabetes (including forms with (1) predominant or profound insulin resistance, (2) predominant insulin deficiency and some insulin resistance and (3) forms intermediate between these), obesity and hyperlipidemia conditions such as hypertriglyceridemia and hypercholesterolemia.

In diabetes, the formulations described herein are useful to (1) enhance β-cell function in the islets of Langerhans (e.g., increase insulin secretion), (2) reduce the rate of islet cell damage, (3) increase insulin receptor levels or activity to increase cell sensitivity to insulin and/or (4) modulate glucocorticoid receptor activity to decrease insulin resistance in cells that are insulin resistant.

Conditions related to autoimmunity that can be treated with the 3β-tetrol solid state forms disclosed herein include Type 1 diabetes (including Immune-Mediated Diabetes Mellitus and Idiopathic Diabetes Mellitus), multiple sclerosis, optic neuritis, Crohn's disease (regional enteritis), ulcerative colitis, rheumatoid arthritis and Hashimotos' thyroiditis.

The solid state forms of 3β-tetrol described herein are thus useful to treat, prevent, ameliorate or slow the progression of conditions or their related symptoms related to or associated with unwanted inflammation that may be acute or chronic.

Formulations useful to treat, prevent, ameliorate or slow the progression of an inflammation, metabolic or autoimmune condition or a symptom associated thereto include formulations comprising one or more excipients and a crystalline hydrate of 3β-tetrol, including Form Iβ.

Other formulations useful to treat, prevent, ameliorate or slow the progression of an inflammation, metabolic or autoimmune condition or a symptom associated thereto include formulations comprising one or more excipients and a crystalline hydrate of 3β-tetrol, including Form IIβ, Form IIIβ, Form IVβ, Form Vβ or Form VIβ3β-tetrol or a mixture thereof substantially free or essentially free of Form Iβ or wherein Form Iβ is absent or is a minor component relative to the total crystalline content of 3β-tetrol.

Additional formulations useful to treat, prevent, ameliorate or slow the progression of an inflammation, metabolic or autoimmune condition or a symptom associated thereto include formulations comprising one or more excipients and a crystalline anhydrate of 3β-tetrol, including Form VIIβ, Form VIIIβ, Form IXβ or Form Xβ 3β-tetrol or a mixture thereof substantially free or essentially free of Form Iβ or wherein Form Iβ is absent or is a minor component relative to the total crystalline content of 3β-tetrol.

Other embodiments of the invention are directed to a particular crystalline form of 3β-tetrol (e.g., Form Iβ, Form IIβ, Form IIIβ, Form IVβ, Form Vβ, Form VIβ, Form VIIβ, Form VIIIβ, Form IXβ or Form Xβ) substantially free or essentially free of other solid state forms of 3β-tetrol.

Additional embodiments of the invention are directed to a particular crystalline hydrate or pseudopolymorph of 3β-tetrol (e.g., Form Iβ, Form IIβ, Form Iβ, Form IIIβ, Form IVβ, Form Vβ or Form VIβ, or a solid state form of 3β-tetrol comprising a mixture of two or more such crystalline hydrates, substantially free or essentially free of other solid state forms of 3β-tetrol.

Additional embodiments of the invention are directed to a particular crystalline anhydrate or polymorph of 3β-tetrol (e.g., Form VIIβ, Form VIIIβ, Form IXβ or Form Xβ) or a mixture of two or three such crystalline anhydrates substantially free or essentially free of other solid state forms of 3β-tetrol or a solid state form of 3β-tetrol comprising a crystalline anhydrate of 3β-tetrol and one or more of its crystalline hydrates.

Preferred formulations comprising a 3β-tetrol solid state form for treating an inflammation, metabolic or autoimmune condition or a symptom associated thereto are for oral administration, optionally in unit dosage forms from 1 mg to 200 mg 3β-tetrol and with Form Iβ and Form VIβ 3β-tetrol preferred.

Other embodiments of the invention are directed to methods of preparation of a particular crystalline form of 3β-tetrol disclosed herein.

In some embodiments a solid state form of 3β-tetrol is characterized or identified by methods comprising X-ray Powder Diffraction (XRPD) and one or more thermal methods including Differential Thermal Analysis (DTA), Differential Scanning calorimetry (DSC), Modulated Differential Scanning calorimetry (mDSC), Thermogravimetric Analysis (TGA), Thermogravimetric-infrared (TG-IR) analysis and melting point measurements.

In some embodiments a solid state form of 3β-tetrol is characterized or identified by methods including XRPD and a vibrational spectroscopy method such as Raman spectroscopy.

Other embodiments of the invention are directed to pharmaceutically acceptable formulations in solid form comprising a particular crystalline form of 3β-tetrol disclosed herein that is substantially free of other crystalline forms of 3β-tetrol and methods for preparation of the formulations.

Still other embodiments of the invention are directed to liquid formulations or invention compositions prepared by contacting or admixing at least one crystalline form of 3β-tetrol with a liquid excipient, optionally in the presence of another excipient, and methods for preparation of the liquid formulation.

Other embodiments that are related to contacting or admixing at least one solid state form of 3β-tetrol with a liquid excipient are directed to solid formulations as suspension formulation wherein at least some amount of 3β-tetrol is present as particles in the formulation. These suspension formulations are made using a solid state form described herein.

Another embodiment of the invention is directed to methods for treating a condition related to hyperglycemia or unwanted inflammation associated with a chronic disease or condition, e.g., an autoimmune condition in a subject, with a solid formulation comprising a solid state form of 3β-tetrol such as a crystalline form of 3β-tetrol.

Other embodiments of the invention are directed to uses of 3β-tetrol in solid state form (e.g., Form Iβ, Form IIβ, Form IIIβ, Form IVβ, Form Vβ, Form VIβ, Form VIIβ, Form VIIIβ, Form IXβ, Form Xβ 3β-tetrol or a mixture thereof) to prepare a medicant for treatment of unwanted inflammation in a subject.

Still other invention embodiments include methods of treating a pathological condition or one or more symptoms of a pathological condition associated with acute or chronic, non-productive inflammation using 3β-tetrol in crystalline form or a formulation or invention composition comprising this crystalline form.

Thus, additional embodiments of the invention include methods of treating a number of clinical conditions or symptoms thereof that are associated with acute inflammation, or tissue damage from such conditions, which may be acute or chronic, with crystalline forms of 3β-tetrol as described herein, or solid or liquid formulations derived therefrom. These acute conditions include acute respiratory disease syndrome and acute asthma, traumas such as chemical burns, thermal burns, radiation burns and reperfusion injury such as myocardial and cerebral infarction.

Other additional embodiments of the invention include methods of treating a number of clinical conditions or symptoms thereof that are associated with chronic inflammation or tissue damage from such conditions, which may be acute or chronic, with crystalline forms of 3β-tetrol as described herein, or solid or liquid formulations derived therefrom. These chronic conditions include inflammatory bowel syndrome or an autoimmune condition such as an arthritis condition, ulcerative colitis or Crohn's disease. These chronic conditions also include cystic fibrosis, chronic bronchitis, chronic obstructive pulmonary disease (COPD) and acute respiratory distress syndrome.

In one preferred embodiment a solid state form of 3β-tetrol is used to treat a metabolic condition or an autoimmune condition in a subject such as a human or other mammal.

In another preferred embodiment a solid state form of 3β-tetrol is used to treat Type 2 diabetes or ulcerative colitis or other metabolic or autoimmune condition.

In certain embodiments, the present invention encompasses the use of the solid state forms of the invention for preparing a final drug product. Preferred drug products are generally prepared using a crystalline hydrate of 3β-tetrol such as Form Iβ or Form VIβ.

Additional embodiments and advantages of the present invention are described further in the following detailed description. The claimed agents and methods are also useful to reduce one or more symptoms associated with the conditions described herein. Additionally, the use of the agents and methods described herein can be combined with one or more conventional treatments for each of these disorders.

### BRIEF DESCRIPTION OF THE FIGURES

FIG. 1. X-Ray powder diffraction pattern of Crystalline Form Iβ androst-5-ene-3β,7β,16α,17β-tetrol

FIG. 2. Solid phase Raman Spectrum of Crystalline Form Iβ androst-5-ene-3β,7β,16α,17β-tetrol

FIG. 3. Differential thermal and thermal gravimetric traces of Crystalline Form Iβ androst-5-ene-3β,7β,16α,17β-tetrol

FIG. 4. Differential thermal and thermal gravimetric traces of Crystalline Form IIβ androst-5-ene-3β,7β,16α,17β-tetrol

FIG. 5. X-Ray powder diffraction pattern of Crystalline Form IIIβ androst-5-ene-3β,7β,16α,17β-tetrol

FIG. 6. Solid phase Raman spectrum of Crystalline Form IIIβ androst-5-ene-3β,7β,16α,17β-tetrol

FIG. 7. Differential thermal and thermal gravimetric traces of Crystalline Form IIIβ androst-5-ene-3β,7β,16α,17β-tetrol

FIG. 8. X-Ray powder diffraction pattern of Crystalline Form IVβ androst-5-ene-3β,7β,16α,17β-tetrol

FIG. 9. Solid phase Raman spectrum of Crystalline Form IVβ androst-5-ene-3β,7β,16α,17β-tetrol

FIG. 10. Differential thermal and thermal gravimetric traces of Crystalline Form IVβ androst-5-ene-3β,7β,16α,17β-tetrol

FIG. 11. X-Ray powder diffraction pattern of Crystalline Form Vβ androst-5-ene-3β,7β,16α,17β-tetrol

FIG. 12. Solid phase Raman spectrum of Crystalline Form Vβ androst-5-ene-3β,7β,16α,17β-tetrol

FIG. 13. Differential thermal and thermal gravimetric traces of Crystalline Form Vβ androst-5-ene-3β,7β,16α,17β-tetrol

FIG. 14. X-Ray powder diffraction pattern of Crystalline Form VIβ androst-5-ene-3β,7β,16α,17β-tetrol

FIG. 15. Solid phase Raman spectrum of Crystalline Form VIβ androst-5-ene-3β,7β,16α,17β-tetrol

FIG. 16. Differential thermal and thermal gravimetric traces of Crystalline Form VIβ androst-5-ene-3β,7β,16α,17β-tetrol

FIG. 17. Differential thermal and thermal gravimetric traces of Crystalline Form VIIβ androst-5-ene-3β,7β,16α,17β-tetrol

FIG. 18. X-Ray powder diffraction pattern of Crystalline Form VIIIβ androst-5-ene-3β,7β,16α,17β-tetrol

FIG. 19. Solid phase Raman spectrum of Crystalline Form VIIIβ androst-5-ene-3β,7β,16α,17β-tetrol

FIG. 20. Differential thermal and thermal gravimetric traces of Crystalline Form VIIIβ androst-5-ene-3β,7β,16α,17β-tetrol

FIG. 21. X-Ray powder diffraction pattern of Crystalline Form IXβ androst-5-ene-3β,7β,16α,17β-tetrol

FIG. 22. Solid phase Raman spectrum of Crystalline Form IXβ androst-5-ene-3β,7β,16α,17β-tetrol

FIG. 23. Differential thermal and thermal gravimetric traces of Crystalline Form IXβ androst-5-ene-3β,7β,16α,17β-tetrol

FIG. 24. X-Ray powder diffraction pattern of Crystalline Form Xβ androst-5-ene-3β,7β,16α,17β-tetrol

FIG. 25. Differential thermal and thermal gravimetric traces of Crystalline Form Xβ androst-5-ene-3β,7β,16α,17β-tetrol

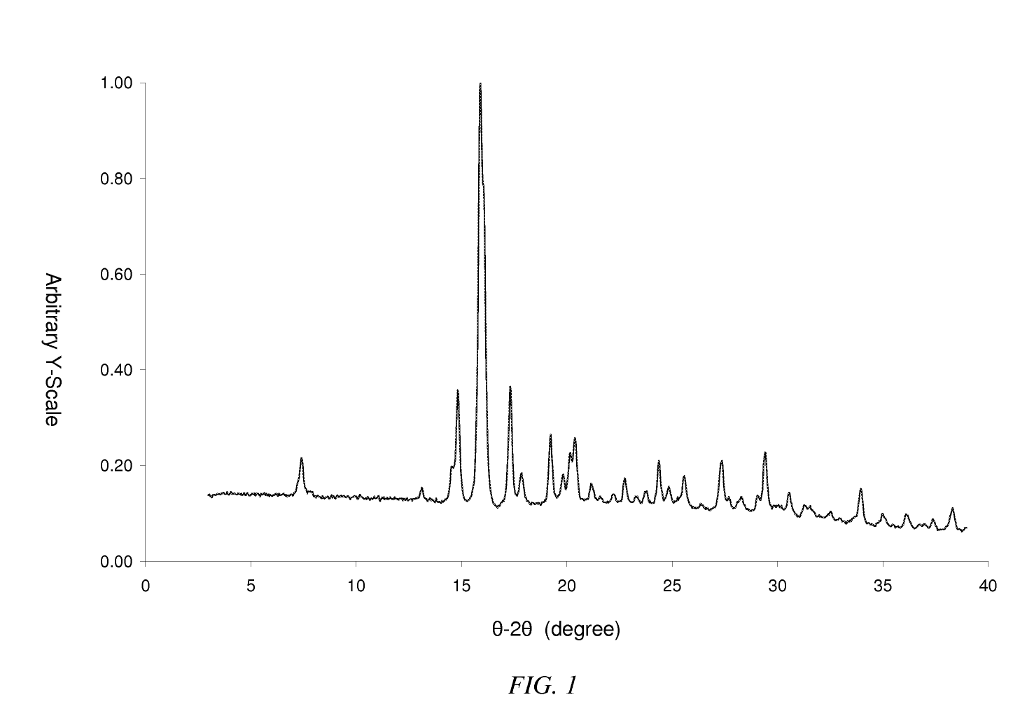

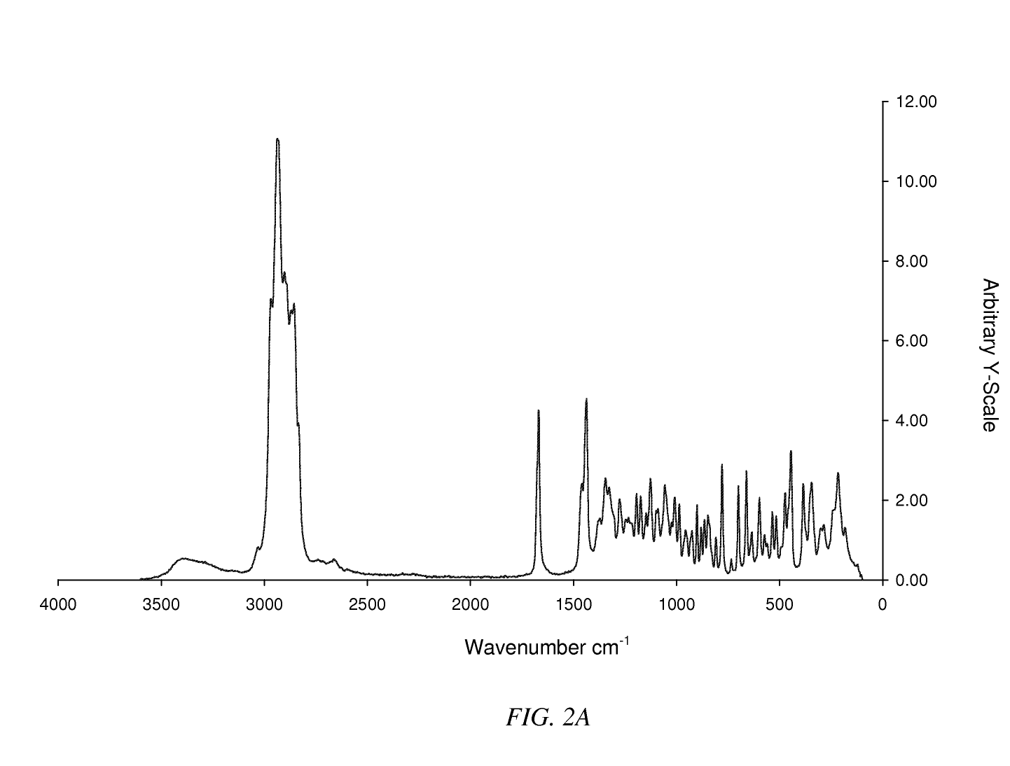

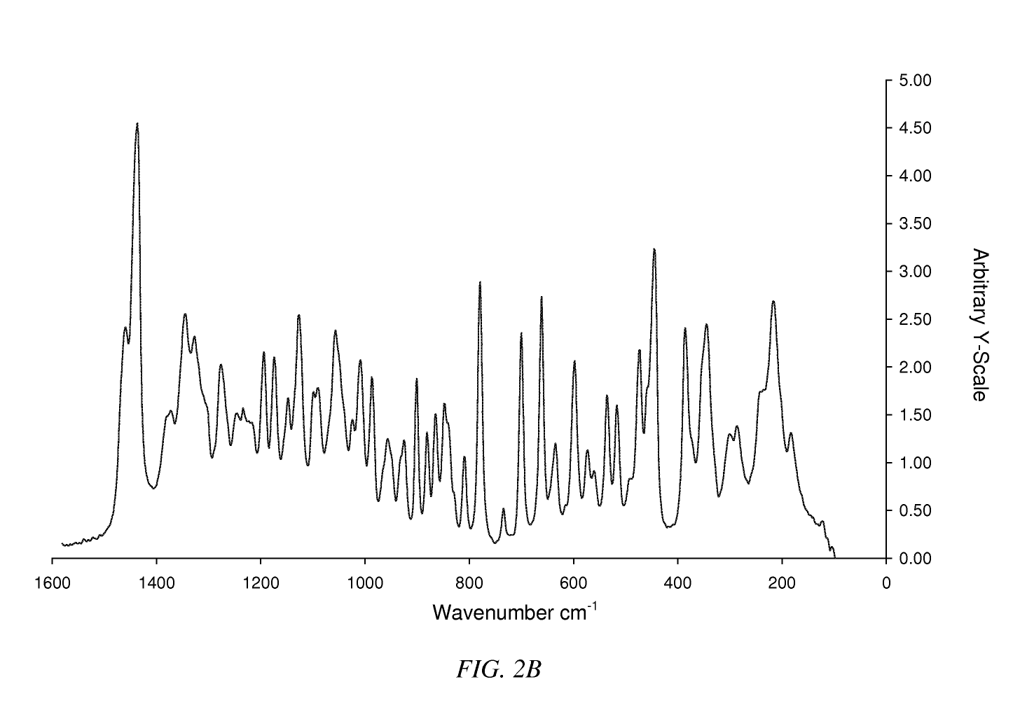

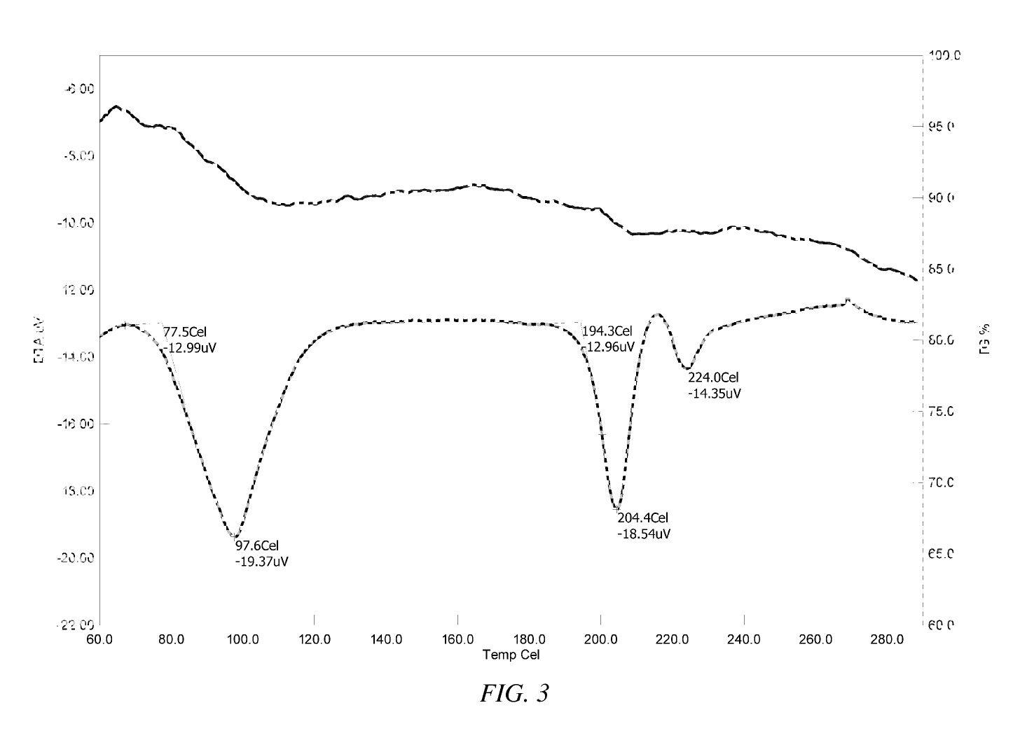

### DETAILED DESCRIPTION

### Definitions

As used herein or otherwise stated or implied by context, terms that are defined herein have the meanings that are specified. The descriptions of embodiments and examples that are described illustrate the invention and they are not intended to limit it in any way. Unless otherwise contraindicated or implied, e.g., by mutually exclusive elements or options, in the descriptions or throughout this specification, the terms “a” and “an” mean one or more and the term “or” means and/or.

Unless specified otherwise explicitly or by context, percentage amounts are expressed as % by weight (w/w). Thus, a solid-dosage formulation containing at least about 2% Compound 1B (i.e., 3β-tetrol) in a solid-dosage formulation or suspension containing at least about 2% w/w 3β-tetrol. A solid solid-dosage formulation of 3β-tetrol containing 0.1% water means 0.1% w/w water is associated with that solid-dosage formulation, excluding water of hydration of a crystalline hydrate that is used to prepare the solid-dosage formulation.

“About” and “approximately,” when used in connection with a numeric value or range of values which is provided to describe a particular solid form, e.g., a specific temperature or temperature range, such as, for example, that describing a melting, dehydration, desolvation or glass transition; a mass change, such as, for example, a mass change as a function of temperature or humidity; a solvent or water content, in terms of, for example, mass or a percentage; or a peak position, such as, for example, in analysis by IR or Raman spectroscopy or XRPD; indicate that the value or range of values may deviate to an extent deemed reasonable to one of ordinary skill in the art while still describing the particular solid state form. Specifically, the terms “about” and “approximately,” when used in this context, indicate that the numeric value or range of values may vary by 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1% or 0.01% of the recited value or range of values while still describing the particular composition or solid state form. For XRPD peaks, the term “about” refers to a variation of ±0.1 or ±0.2 degree 2-theta for sharp, well-defined peaks.

“Solid State” as used herein refers to a physical state of a compound or composition comprising the compound, such as androst-5-ene-3β,7β,16α,17β-tetrol (i.e., 3β-tetrol); wherein at least about 2-10% of the mass of the compound that is present exists as a solid. Typically, the majority of the mass of 3β-tetrol will be in solid state form. More typically, between at least about 80-90% of the mass of 3β-tetrol is in solid form. Solid state forms include crystalline, disordered crystalline, polycrystalline, microcrystalline, nanocrystalline, partially crystalline or mixtures thereof, optionally with non-solid or non-crystalline 3β-tetrol. Solid state forms of Compound 3β-tetrol further include polymorphs, pseudopolymorphs, hydrates, solvates, dehydrated hydrates and desolvated solvates and mixtures thereof, optionally with non-solid or non-crystalline 3β-tetrol. Thus, solid state forms of 3β-tetrol will include a single polymorph form of 3β-tetrol, a single pseudo-polymorph form of 3β-tetrol, a mixture of two or more, typically two or three, polymorph or pseudo-polymorph forms of 3β-tetrol or a combination of any one of these solid state forms, optionally with non-solid or non-crystalline 3β-tetrol, provided that at least about 2-10% of the mass of 3β-tetrol is in solid form.

The term “crystalline” and related terms used herein, when used to describe a substance, component or product, means that the substance, component or product is crystalline as determined by visual inspection or usually with a suitable method, typically an

X-ray diffraction method such as X-ray powder diffraction [See, e.g., Remington's Pharmaceutical Sciences, 18^{th} ed., Mack Publishing, Easton Pa., p173 (1990); The United States Pharmacopeia, 23^{rd} ed., pp. 1843-1844 (1995)].

The term “crystalline forms” and related terms herein refers to the various crystalline modifications of a given substance, including, but not limited to, polymorphs, solvates, hydrates, mixed solvates, co-crystals and other molecular complexes. A crystalline form may also be a mixture various crystalline modifications of a given substance such as a combination of pseudopolymorph or polymorph forms, a combination of one or more polymorph forms with one or more pseudopolymorph or a combination of such forms with non-crystalline or non-solid state forms of the substance. Typical combinations are of two or more polymorph or pseudo polymorph forms, such a mixture of a polymorph form with a pseudopolymorph form or a mixture of a polymorph or pseudopolymorph form with non-crystalline material. Typically crystalline forms are typically distinguishable from each other by their XRPD patterns. Solid state forms having different crystal morphologies but essentially identical XRPD patterns are considered to be different crystalline forms, since different morphologies can exhibit different properties related to physical shape. Properties related to physical shape include dissolution rate, stability, hygroscopicity, mechanical properties such hardness, tensile strength, compatibility (tableting) and those related to handling, e.g., flow, filtering, blending and other physical or pharmaceutical properties as described herein for different polymorphs.

“Polymorph” as used herein refers to a defined crystalline form of androst-5-ene-3β,7β,16β,17β-tetrol (i.e., 3β-tetrol). Polymorphs typically differ in their physical properties due to the order of the molecules in the lattice of the polymorph. Thus, polymorphs may exhibit one or more differences in physical or pharmaceutical properties including hygroscopicity, solubility, intrinsic dissolution rate, solid state reaction rates (i.e., chemical stability of a pharmaceutical ingredient as the drug substance or drug product), crystalline stability (i.e. tendency to transition to a more thermodynamically stable crystalline form), surface free energy, interfacial tension, mechanical strength (e.g., hardness, brittleness, plastic deformation, docility, malleability, etc.), tensile strength, compactability (i.e., tableting) and processability (e.g., handling, flow, blending, etc.). Differences in physical and mechanical properties of polymorphic forms of a drug substance may also affect scale-up and transfer from laboratory procedures though pilot plant and then to full production.

Polymorphs existing as hydrates, solvates or mixed solvates are generally referred to as pseudopolymorphs and represent different polymorphic or solid state forms in view of an isostructural polymorph form that is anhydrous or not a solvate. Pseudopolymorphs that differ in solvate identity or stoichiometry are also considered different polymorphic or solid state forms in view of each other. For example, 3β-tetrol existing as a solvate (e.g., crystalline Form Iβ) is a different solid state form in view of another solvate (e.g., crystalline Form VIβ) or an anhydrate (e.g., crystalline Form VIIIβ). Stability profiles of hydrates and solvates at various temperatures and/or at different vapor pressures of water (e.g., relative humidity) or organic solvents will sometimes differ from those of the isostructural anhydrate or desolvate. Such differences may influence formulation, processing or stability of an active pharmaceutical ingredient (e.g., 3β-tetrol), either as the drug substance in a drug product under various storage conditions.

Thus, different crystalline or polymorphic forms may have different physical properties such as, for example, melting temperatures, heats of fusion, solubilities, and/or vibrational spectra as a result of the arrangement or conformation of the molecules in the crystal lattice (see, e.g., Byrn, S. R., Pfeiffer, R. R., and Stowell, J. G. (1999) Solid-State Chemistry of Drugs, 2^{nd} ed., SSCI, Inc.: West Lafayette, Ind.). The differences in physical properties exhibited by polymorphs and pseudopolymorphs may affect pharmaceutical parameters such as storage stability, compressibility and density (important in formulation and product manufacturing), and dissolution rate, which can be an important factor in bioavailability. Differences in stability may result from changes in chemical reactivity (e.g., differential oxidation, such that a dosage form discolors more rapidly when comprised of one polymorph or pseudopolymorph than when comprised of another polymorphic form) or mechanical changes (e.g., tablets crumble on storage as a kinetically favored polymorph converts to thermodynamically more stable polymorph) or both (e.g., tablets of one polymorph are more susceptible to breakdown at high humidity). As a result of kinetic solubility/dissolution rate differences, in the extreme case, some polymorphic transitions may result in lack of potency or, at the other extreme, toxicity. In addition, the physical properties of the crystal may be important in processing, e.g., one polymorph might be more likely to form solvates or hydrates that may be difficult to filter or wash free of impurities due to, for example, by differences in crystal morphology and/or particle size distribution.

Typically, crystalline forms are distinguished from each other by one or more physical or analytical properties such as rate of dissolution, Infrared and Raman spectroscopy, X-ray diffraction techniques such as single crystal and powder diffraction techniques, solid state-NMR(SS-NMR), thermal techniques such as melting point, differential thermal analysis (DTA), differential scanning calorimetry (DSC), thermal gravimetric analysis (TGA) and other methods as disclosed elsewhere in the specification. Additional methods to characterize or distinguish one pseudopolymorph from another polymorphic form, include elemental analysis, Karl-Fisher titration, dynamic vapor sorption analysis, thermogravimetric-infrared spectroscopic analysis (TG-IR), residual solvent gas chromatography and ¹H-NMR.

The term “isostructural crystalline form,” as used herein, refers to a crystal form of a substance that has a common structural similarity with another crystalline form, including approximately similar interplanar spacing in the crystal lattice. Thus, isostructural crystalline forms will have similar molecular packing motifs, but differing unit cell parameters (a symmetry translation). Due to their common structural similarity, isostructural crystalline forms typically have similar, but not necessarily identical, X-ray powder diffraction patterns. An isostructural crystalline form may be based upon a substance that is a neutral molecule or a molecular complex. The isostructural crystalline form may be a solvate, including a hydrate, or a desolvated solvate crystalline form of the substance. Isostructural forms that are solvates of a polymorph are sometimes referred to as pseudopolymorphic to the unsolvated polymorph. A solvated crystalline form typically contains one or more solvents, including water, in the crystal lattice, that may be the solvent or solvents of crystallization used in preparing the crystalline form.

“Formulation”, “pharmaceutical formulation” or “pharmaceutically acceptable formulation” as used herein refers to a composition comprising androst-5-ene-3β,7β,16β,17β-tetrol (i.e., 3β-tetrol), present in a solid state form, in addition to one or more pharmaceutically acceptable excipients. Formulations preferably are compositions prepared from a solid state form of 3β-tetrol, wherein the composition is suitable for administration to a human. The formulation may be comprised of or prepared from a crystalline form of 3β-tetrol, or be prepared from, one, two or more crystalline forms of 3β-tetrol, e.g. a single polymorph or pseudopolymorph form of 3β-tetrol, a mixture of two polymorph forms or pseudopolymorph forms of 3β-tetrol or a mixture of a polymorph form of 3β-tetrol and a pseudopolymorph form of 3β-tetrol.

Formulations comprising or prepared from a solid state form of 3β-tetrol, wherein the solid state from is a crystalline form, include Form IIβ, substantially free or essentially free of one or more solid state forms optionally selected from the group consisting of Form Iβ, Form IIIβ, Form IVβ, Form Vβ, Form VIβ, Form VIIβ Form VIIIβ, Form IXβ and Form Xβ.

Other formulations comprising or prepared from a solid state form of 3β-tetrol, wherein the solid state from is a crystalline form, include Form IIIβ substantially free or essentially free of one or more solid state forms optionally selected from the group consisting of Form Iβ, Form IIβ, Form IVβ, Form Vβ, Form VIβ, Form VIIβ Form VIIIβ, Form IXβ and Form Xβ.

Additional formulations comprising or prepared from a solid state form of 3β-tetrol, wherein the solid state from is a crystalline form, include Form IVβ substantially free or essentially free of one or more solid state forms optionally selected from the group consisting of Form Iβ, Form IIβ, Form IIIβ, Form Vβ, Form VIβ, Form VIIβ, Form VIIIβ, Form IXβ and Form Xβ.

Other formulations comprising or prepared from a solid state form of 3β-tetrol, wherein the solid state from is a crystalline form, include Form Vβ substantially free or essentially free of one or more solid state forms optionally selected from the group consisting of Form Iβ, Form IIβ, Form IIIβ, Form IVβ, Form VIβ, Form VIIβ, Form VIIIβ, Form IXβ and Form Xβ.

Other formulations comprising or prepared from a solid state form of 3β-tetrol include Form VIβ substantially free or essentially free of one or more solid state forms optionally selected from the group consisting of Form Iβ, Form IIβ, Form IIIβ, Form IVβ, Form Vβ, Form VIIβ, Form VIIIβ, Form IXβ and Form Xβ;

Additional formulations comprising or prepared from a solid state form of 3β-tetrol, wherein the solid state from is a crystalline form, include Form VIIβ substantially free or essentially free of one or more solid state forms optionally selected from the group consisting of Form Iβ, Form IIβ, Form IIIβ, Form IVβ, Form Vβ, Form VIβ, Form VIIIβ, Form IXβ and Form Xβ.

Other formulations comprising or prepared from a solid state form of 3β-tetrol, wherein the solid state from is a crystalline form, include Form VIIIβ substantially free of one or more solid state forms optionally selected from the group consisting of Form Iβ, Form IIβ, Form IIIβ, Form IVβ, Form Vβ, Form VIβ, Form VIIβ, Form IXβ and Form Xβ;

Additional formulations comprising or prepared from a solid state form of 3β-tetrol, wherein the solid state from is a crystalline form, include Form IXβ substantially free of one or more solid state forms selected from the group consisting of Form Iβ, Form IIβ, Form IIIβ, Form IVβ, Form Vβ, Form VIβ, Form VIIIβ and Form Xβ;

Other formulations comprising or prepared from a solid state form of 3β-tetrol, wherein the solid state from is a crystalline form, include Form Xβ substantially free of one or more solid state forms optionally selected from the group consisting of Form Iβ, Form IIβ, Form IIIβ, Form IVβ, Form Vβ, Form VIβ, Form VIIβ, Form VIIIβ and Form IXβ.

Typically, formulations of 3β-tetrol will be comprised of or prepared from one crystalline form of 3β-tetrol (e.g., crystalline Form Iβ, Form IIβ, Form IIIβ, Form IVβ, Form Vβ, Form VIβ, Form VIIβ, Form VIIIβ, Form IXβ or Form Xβ) or, less preferably, a mixture of two or more crystalline hydrate forms selected from the group consisting of Form Iβ, Form IIβ, Form IIIβ, Form IVβ, Form Vβ and Form VIβ, a mixture of crystalline anhydrate forms selected from the group consisting of Form VIIβ, Form VIIIβ, Form IXβ and Form Xβ or a mixture of one or more crystalline hydrate forms selected from the group consisting of Form Iβ, Form IIβ, Form IIIβ, Form IVβ, Form Vβ and Form VIβ with Form VIIβ, Form VIIIβ, Form IXβ or Form Xβ.

Preferred formulations of 3β-tetrol contain Form Iβ or Form VIβ predominately free or essentially free of other solid state forms of 3β-tetrol.

“Solid formulation” as used herein refers to a pharmaceutically acceptable formulation wherein 3β-tetrol is in solid state form in the presence of one or more pharmaceutically acceptable excipients wherein the majority of the mass amount of the solid state form of 3β-tetrol used in preparation of the formulation remains in that solid state form. Solid formulations will preferably remain solid for at least about 6 months at ambient temperature, usually for at least about 12 months or 24 months at ambient temperature, when mixed or combined with the excipients in proportions required for the solid state formulation. Dosage units that are a solid formulation include tablets, capsules, caplets, gelcaps, suspensions and other dosage units typically associated with oral administration of an active pharmaceutical ingredient in solid state form to a subject in need thereof. Unit dosages are preferably tablets, capsules or gelcaps.

“Liquid formulation” as used herein refers to a pharmaceutically acceptable formulation wherein one or more solid state forms of 3β-tetrol has been admixed or contacted with one or more pharmaceutically acceptable excipients, wherein at least one of the excipients is in liquid state form in proportions required for the liquid formulation, such that a majority of the mass amount of 3β-tetrol is dissolved into the non-solid excipient. Dosage units containing a liquid formulation include syrups, gels, ointments and other dosage units typically associated with parenteral or enteral administration of an active pharmaceutical ingredient to a subject in need thereof in non-solid state form.

“Suspension formulation” as used herein refers to a pharmaceutically acceptable formulation wherein one or more solid state forms of 3β-tetrol has been admixed or contacted with one or more pharmaceutically acceptable excipients, wherein at least one of the excipients is in liquid or non-solid state form (i.e. a non-solid excipient), in proportions wherein the majority of the mass amount of 3β-tetrol is not dissolved or is suspended in the non-solid state excipient or the excipient mixture of which the non-solid state excipient is comprised.

“Invention composition” as used herein refers to a mixture comprised of or prepared from one or more solid state forms of 3β-tetrol and one or more other components. Thus, an invention composition may be comprised of or prepared from one or more solid state forms of 3β-tetrol and one or more excipients and is a composition that may or may not be suitable for administration to a subject. In some embodiments, an invention composition consists essentially of pharmaceutically acceptable excipients and a solid state form of 3β-tetrol and may or may not require addition of another pharmaceutically acceptable excipient prior to administration to a subject by an intended route of delivery. For example, a lyophilized formulation containing or prepared from a solid state from of 3β-tetrol will typically require addition of a suitable liquid excipient prior to parenteral delivery by injection to a subject.

“Substantially free” as used herein refers to a compound such as 3β-tetrol wherein more than about 60% by weight of the compound is present as the given solid state form. For example, the term crystalline 3β-tetrol “substantially free” of amorphous material refers to a solid-state form of 3β-tetrol wherein more than about 60% of 3β-tetrol is in one or more crystalline forms. Such compositions preferably contain at least about 80%, more preferably at least about 90%, of 3β-tetrol in one or more crystalline forms with the remaining present as non-crystalline 3β-tetrol. In yet another example, the term Form Iβ “substantially free” of other crystalline forms refers to a solid-state composition of 3β-tetrol wherein more than about 60% of 3β-tetrol exists as Form Iβ. Such compositions typically contain at least about 80%, preferably at least about 90%, more preferably at least about 95% 3β-tetrol as a single crystalline form. Preferred formulations of 3β-tetrol contain at least about 80%, preferably at least about 90% and more preferably at least about 95% of 3β-tetrol as Form Iβ or Form VIβ, with the remaining 3β-tetrol present as other solid state or non-solid state forms. Most preferred formulations contain about 95-99% of Form Iβ 3β-tetrol or Form VIβ 3β-tetrol with about 97%, about 98% or about 99% as a single crystalline form of 3β-tetrol particularly preferred.

“Essentially free” as used herein refers to a component so identified as not being present in an amount that is detectable under typical conditions used for its detection or would adversely affect the desired properties of a composition or formulation in which the component may be found. For example, “essentially free of liquid” means a composition or formulation in solid form that does not contain water or solvent, in liquid form, in an amount that would adversely affect the pharmaceutical acceptability of the formulation or composition for use in a solid dosage form to be administered to a subject in need thereof. A suspension is considered a solid formulation and for such formulations liquid excipient(s) comprising the suspension formulation are not included within this definition.

“Substantially pure” as used herein refers to a solid state form of 3β-tetrol that contain less than about 3% or less than about 2% by weight total impurities, or more preferably less than about 1% by weight water, and/or less than about 0.5% by weight impurities such as decomposition or synthesis by-products or residual organic solvent. Residual solvent does not include solvent that is part of a crystalline solvate (i.e., a pseudopolymorph) such as water in a crystalline hydrate of 3β-tetrol.

“Substantially identical” as used herein refers to measured physical characteristics that are comparable in value or data traces that are comparable in peak position and amplitude or intensity within the scope of variations that are typically associated with sample positioning or handling or the identity of the instrument employed to acquire the traces or physical characteristics or due to other variations or fluctuations normally encountered within or between laboratory environments or analytical instrumentation.

“Hydrate” as used here refers to a solid state form of a compound such as 3β-tetrol that contains water molecules as an integral part of the solid state form and does not refer to water that is non-specifically bound to the bulk compound. Hydrates in a crystalline form can be isolated site hydrates or channel hydrates. Hydrates can contain stoichiometric or nonstoichiometric amounts of water molecules per compound molecule. Typically, water will be present in a crystalline hydrate in the ratio of 0.25, 0.5, 1.0, 1.5 or 2.0 relative to the compound of the crystalline hydrate on a mole basis.

“Solvate” as used here refers to a solid state form of a compound such as 3β-tetrol that contains solvent molecules as an integral part of the solid state form and does not refer to solvent that is non-specifically bound to bulk compound. When the solvent molecule is water such solvates are sometimes referred herein as hydrates.

“Inflammation condition” as used herein refers to a condition that is characterized by the inappropriate or pathological presence of inflammation or its associated pain or fever. Inflammation may be acute or chronic and is present in metastatic cancer, e.g., metastatic prostate or breast cancer, metabolic and cardiovascular conditions such as Type 2 diabetes and atherosclerosis, and autoimmune conditions such as ulcerative colitis. Acute inflammation may be present as a flare as for example in multiple sclerosis.

Inflammation conditions include autoimmune conditions, such as multiple sclerosis, a lupus condition, e.g., systemic lupus erythematosus (an autoimmune condition), an arthritis condition, e.g., rheumatoid arthritis (an autoimmune condition), and an inflammatory bowel condition, e.g., ulcerative colitis or Crohn's disease (autoimmune conditions). Inflammation conditions also include metabolic conditions, such as hyperglycemia conditions, diabetes, liver inflammation conditions, e.g., nonalcoholic steatohepatitis (NASH) and nonalcoholic fatty liver disease. Inflammation conditions also include acute and chronic lung inflammation conditions, e.g., obstructive pulmonary disease (COPD), acute asthma, chronic asthma, emphysema, acute bronchitis, allergic bronchitis, chronic bronchitis and lung fibrosis. Inflammation conditions further include neuroinflammation in neurodegenerative conditions such as Alzheimer's disease, Parkinson's disease, amyotrophic lateral sclerosis and age-related macular degeneration.

“Metabolic condition” as used herein include type 1 diabetes (an autoimmune condition), type 2 diabetes, obesity, metabolic syndrome, insulin resistance, hyperglycemia, impaired glucose utilization or tolerance, impaired or reduced insulin synthesis, a hyperlipidemia condition, such as hyperlipidemia, hypercholesterolemia, hypertriglyceridemia, elevated free fatty acids, or macrovascular damage, such as arterial atherosclerosis, hypolipidemias or vascular atherosclerosis. Hypercholesterolemia includes hyper-LDL cholesterolemia or elevated LDL cholesterol. Hypolipidemias include hypo-HDL cholesterolemia or low HDL cholesterol levels. Type 1 diabetes includes Immune-Mediated Diabetes Mellitus and Idiopathic Diabetes Mellitus. Type 2 diabetes includes forms with predominant or profound insulin resistance, predominant insulin deficiency and some insulin resistance and forms intermediate between these.

An “excipient”, “carrier”, “pharmaceutically acceptable carrier” or similar terms mean one or more component(s) or ingredient(s) that is acceptable in the sense of being compatible with the other ingredients in formulations or invention compositions comprising 3β-tetrol as the active pharmaceutical ingredient that is in solid state form when admixed with one or more of the excipients. These excipients usually are not overly deleterious to a subject to whom the composition formulation is to be administered. Excipients include one or more components typically used in the pharmaceutical formulation arts, e.g., one, two or more of fillers, binders, disintegrants, dispersants, preservatives, glidants, surfactants and lubricants. Exemplary excipients include povidone, crospovidone, corn starch, carboxymethyl cellulose, hydroxypropyl methylcellulose, microcrystalline cellulose, gum arabic, polysorbate 80, butylparaben, propylparaben, methylparaben, BHA, EDTA, sodium lauryl sulfate, sodium chloride, potassium chloride, titanium dioxide, magnesium stearate, castor oil, olive oil, vegetable oil, buffering agents such as sodium hydroxide, monobasic sodium phosphate, dibasic sodium phosphate, potassium hydroxide, monobasic potassium phosphate, dibasic potassium phosphate, tribasic potassium phosphate, potassium carbonate, potassium bicarbonate, ammonium hydroxide, ammonium chloride, saccharides such as mannitol, glucose, fructose, sucrose or lactose.

A “subject” means a human or an animal. Usually the animal is a mammal or vertebrate such as a non-human primate, dog or rodent.

A “surface-active agent” (surfactant) means a substance, which, at low concentrations, interacts between the surfaces of a solid and fluid in which the solid is insoluble or sparingly soluble. The fluid may be a liquid excipient present in a suspension formulation that comprises a solid state form of an active pharmaceutical ingredient, such as a solid state form of 3β-tetrol, the liquid excipient and a surface active agent that acts to improve suspendability.

Alternatively, the surface active agent may be present in an oral solid dosage form comprising as the active pharmaceutical ingredient a polymorph or pseudopolymorph of 3β-tetrol (e.g., crystalline Form Iβ, Form IIβ, Form IIIβ Form IVβ, Form Vβ, Form VIβ, Form VIIβ, Form VIIIβ, Form IXβ or Form Xβ) or a mixture thereof and the surface active agent, which acts to improve dissolution rate of the active pharmaceutical ingredient in gastric fluid. Surface-active agents are amphipathic in structure having both polar (hydrophilic) and non-polar (hydrophobic) regions in the same molecule. Examples of surface active agents used in the formulation arts are given in Corrigan, O. I.; Healy, A. M. “Surfactants in Pharmaceutical Products and Systems” in Encyclopedia of Pharmaceutical Technology 2^{nd} ed. Taylor and Francis, 2006, pp. 3583-3596.

A “suspension” generally refers to a solid state form of 3β-tetrol that is present, usually as a finely divided (e.g., micronized) crystalline solid, in a liquid carrier (vehicle) at a time prior to administration of the suspension. The suspension may be either ready to use or a dry powder reconstituted as a suspension dosage form just prior to use. Suspensions typically include a suspending or flocculating agent, a wetting agent, if the suspending or flocculating agent that is present does not already serve this purpose. In a colloidal suspension, the 3β-tetrol particles are typically less than about 1 μm in size. In a coarse suspension, they are larger than about 1 μm. The practical upper limit for individual suspendable particles in coarse suspensions is about 50 μm to 75 μm although some proportion of particles up to 200 μm may be suitable dependent upon the syringeability of the suspension. Design considerations for developing a suspension for oral or parenteral administration are given in Akers, et al. J. Parenteral Sci. Tech. 1987 Vol. 41, pp. 88-96; Nash, R A “Suspensions” in Encyclopedia of Pharmaceutical Technology 2^{nd} ed. Taylor and Francis, 2006, pp 3597-3610 (which is hereby incorporated by reference herein).

Treatment Methods

Chronic, non-productive inflammation is the inappropriate presence of an unresolved inflammatory response that may become disconnected from any stimulus that may have been responsible for initiating the response. The unresolved inflammatory response may be present at an asymptomatic and chronically low level that worsens to a disease state or be symptomatic with occasional, unexpected intensification (e.g., a flare as in an autoimmune disease such as multiple sclerosis). Oftentimes, the unresolved inflammation transforms into a disease state that can be further perpetuated by the underlying inflammation (e.g. metabolic syndrome transitioning to type 2 diabetes). In some instances, a disease state is established irrespective of the initial presence of any inflammatory response, but is perpetuated by an inflammatory response produced by the disease state that becomes chronic (e.g. Alzheimer's disease). In other instances an established unresolved inflammatory response produced by or associated with a disease state sequel can sometimes progress into another disease state with more serious consequences (e.g., ulcerative colitis transitioning to colon cancer) or it may assist in worsening or propagating the disease state (e.g., promoting tumor metastasis in cancer).

As a result, there is the growing realization in the scientific community that unresolved inflammation underlies what on the surface appears to be a disparate collection of disease states and include conditions associated with chronic or acute non-productive inflammation or tissue loss or damage from these conditions.

The chronic or acute inflammatory-based diseases or conditions described herein to be treated with 3β-tetrol or solid or liquid formulations or invention compositions derived therefrom, may be mild and relatively newly diagnosed or more progressed and moderate to severe. In moderately to severely affected patients 3β-tetrol will typically slow the progression of the condition or ameliorate one or more symptoms such as memory loss, dementia, fever, pain or insulin resistance.

Symptoms and treatment effects for chronic or acute inflammatory-based diseases or conditions described herein, include, e.g., reduced abdominal pain, bleeding or tissue damage associated with an inflammatory bowel disease, which may be associated with progression of the condition or intestinal tissue damage,

Other symptoms and treatment effects include decreased hyperglycemia in type 2 diabetes patients, type 1 diabetes patients or obese or hyperglycemic pre-diabetic patients who may be prone to developing diabetes,

Additional symptoms and treatment effects include decreased mood swings, confusion, depression, agitation, short term memory impairment or insulin resistance in patients diagnosed with Alzheimer's disease or other neurological disorders.

Still other symptoms and treatment effects include reduced fatigue, weakness or liver tissue damage or reduced elevation of liver enzyme(s) (AST, SGOT, ALT, SGPT) in liver fibrosis as, for example, in non-alcoholic steatohepatitis (NASH), nonalcoholic fatty liver disease or hepatitis, e.g., viral hepatitis and liver cirrhosis, e.g., alcoholic cirrhosis, which enzyme(s) elevation may be asymptomatic or not.

Similar effects are also expected in patients having a symptom associated with acute inflammation in, for example, a bone fracture or a stroke, e.g., reduced pain or tissue damage.

Treatment of patients having one of the clinical conditions described herein will typically begin after the condition has been diagnosed, but the treatment can also be prophylactic and started when a patient is considered to be susceptible to developing a given condition, e.g., elderly patients having some age-associated memory loss or other cognitive impairment or patients having early stage Alzheimer's disease with limited dementia can be treated to slow the progression or delay the onset of the condition or to limit the severity of a symptom(s). Such treatment or prophylactic effects may be observed by comparison with untreated patients having a similar age, gender, medical history and/or disease profile or condition.

Conditions of unresolved inflammation to be treated with a crystalline form of androst-5-ene-3β,7β,16α,17β-tetrol or a formulation or invention composition comprising or derived from the crystalline from (i.e., solid or liquid formulations prepared using a crystalline form of 3β-tetrol), include metabolic conditions, autoimmune conditions, lung inflammation conditions, inflammatory bowel conditions, neurodegenerative conditions and hyperproliferation conditions described herein.

Conditions of unresolved acute inflammation to be treated with a crystalline form of androst-5-ene-3β,7β,16α,17β-tetrol or a formulation or invention composition comprising or derived from the crystalline from (i.e., solid or liquid formulations prepared using a crystalline form of 3β-tetrol), include ischemic conditions or reperfusion injury, chemical or thermal burns and bone loss or bone damage conditions described herein.

Autoimmune conditions to be treated with a crystalline form of 3β-tetrol or a formulation derived therefrom include a lupus condition such as systemic lupus erythematosus and discoid lupus, rheumatoid arthritis, multiple sclerosis, myasthenia gravis, Graves disease, Sjögren's syndrome, Hashimotos thyroiditis, Crohn's disease and type 1 diabetes. In preferred embodiments these autoimmune conditions are treated using Form Iβ 3β-tetrol. In other preferred embodiments these conditions are treated using Form VIβ 3β-tetrol. In additional preferred embodiments these conditions are treated using Form VIIβ 3β-tetrol or Form VIIIβ 3β-tetrol.

Lung inflammation conditions to be treated with a crystalline form of 3β-tetrol, or a formulation derived therefrom, include chronic obstructive pulmonary disease (COPD), acute asthma, chronic asthma, emphysema, acute bronchitis, allergic bronchitis, allergic respiratory disease, chronic bronchitis, pleurisy, allergic bronchopulmonary aspergillosis, chronic interstitial pneumonia, respiratory bronchiolitis-associated interstitial lung disease, cystic fibrosis, or fibrosing alveolitis (lung fibrosis) conditions such as subepithelial fibrosis in patients having chronic bronchitis. In preferred embodiments these lung inflammation conditions are treated using Form Iβ 3β-tetrol. In other preferred embodiments these conditions are treated using Form VIβ 3β-tetrol. In additional preferred embodiments these conditions are treated using Form VIIβ 3β-tetrol or Form VIIIβ 3β-tetrol.

Metabolic conditions to be treated with a crystalline form of 3β-tetrol, or a formulation derived therefrom, include metabolic syndrome, Type 2 diabetes, Type 1 diabetes, hyperglycemia, hyperlipidemia, hypertriglyceridemia, hypercholesterolemia, liver cirrhosis conditions and nonalcoholic steatohepatitis (NASH), nonalcoholic fatty liver disease or other fatty liver conditions. In preferred embodiments these metabolic conditions are treated using Form Iβ 3β-tetrol. In other preferred embodiments these conditions are treated using Form VIβ 3β-tetrol. In additional preferred embodiments these conditions are treated using Form VIIβ 3β-tetrol or Form VIIIβ 3β-tetrol.

Inflammatory bowel conditions to be treated with a crystalline form of 3β-tetrol, or a formulation derived therefrom, include ulcerative colitis, Crohn's disease or inflammatory bowel syndrome (IBS). In preferred embodiments these inflammatory bowel condition conditions are treated using Form Iβ 3β-tetrol. In other preferred embodiments these conditions are treated using Form VIβ 3β-tetrol. In additional preferred embodiments these conditions are treated using Form VIIβ 3β-tetrol or Form VIIIβ 3β-tetrol.

Neurodegenerative diseases to be treated, by slowing progression of the disease or reducing inflammation, using a crystalline form of 3β-tetrol, or a formulation derived therefrom, include Alzheimer's disease, Parkinson's disease, dementias or a cognitive impairment condition without dementia, Huntington's disease or Amyotrophic lateral sclerosis (ALS). In preferred embodiments these neurodegenerative diseases are treated using Form Iβ 3β-tetrol. In other preferred embodiments these conditions are treated using Form VIβ 3β-tetrol. In additional preferred embodiments these conditions are treated using Form VIIβ 3β-tetrol or Form VIIIβ 3β-tetrol.

Hyperproliferation conditions or cancers to be treated, by, e.g., slowing progression of the disease, using a crystalline form of 3β-tetrol, or a formulation derived therefrom, include breast cancer, prostate cancer and hyperplasia conditions such as benign prostatic hyperplasia. In preferred embodiments these hyperproliferation conditions are treated using Form Iβ 3β-tetrol. In other preferred embodiments these conditions are treated using Form VIβ 3β-tetrol. In additional preferred embodiments these conditions are treated using Form VIIβ 3β-tetrol or Form VIIIβ 3β-tetrol.

Acute, non-productive inflammation conditions or tissue damage from these conditions to be treated with a crystalline form of 3β-tetrol, or a formulation derived therefrom, include skin lesions or disruptions, e.g., associated with wounds, keratosis or psoriasis, an ischemia condition, e.g., myocardial infarction, stroke and other central nervous system ischemia conditions such as brain hemorrhage, thromboembolism and brain trauma, bone loss or damage conditions, e.g., osteoarthritis and osteoporosis conditions such as postmenopausal osteoporosis, idiopathic osteoporosis or osteoporosis associated with treatment with a glucocorticoid, e.g., dexamethasone, prednisone, cortisone or corticosterone. In preferred embodiments these acute conditions are treated using Form Iβ 3β-tetrol. In other preferred embodiments these conditions are treated using Form VIβ 3β-tetrol. In additional preferred embodiments these conditions are treated using Form VIIβ 3β-tetrol or Form VIIIβ 3β-tetrol.

A number of factors may contribute to the establishment and maintenance of some of the chronic inflammation conditions described herein with production of pro-inflammatory cytokines and chemokines being a common feature. For example, tumor necrosis factor-α (TNF-α) is a cytokine that is released primarily by mononuclear phagocytes in response to a number of immuno-stimulators. When administered to animals or humans, it causes inflammation, fever, cardiovascular effects, hemorrhage, coagulation, and acute phase responses similar to those seen during acute infections and shock states. Normal TNF-αlevels are needed to elicit a number of normal immune responses. Excessive or unregulated TNF-αproduction may play a role in a number of disease conditions. These conditions include endotoxemia and/or toxic shock syndrome, e.g., Tracey et al., Nature 330:662-664 (1987) and Hinshaw et al., Circ. Shock 30: 279-292 (1990), cachexia, e.g., Dezube et al., Lancet, 335(8690): 662 (1990) and acute respiratory distress syndrome (ARDS) where high TNF-αconcentrations have been detected in pulmonary aspirates from ARDS patients, e.g., Millar et al., Lancet 2(8665): 712-714 (1989).

Excessive levels of TNF-αalso may be involved in bone resorption diseases, including arthritis. When activated, leukocytes can produce bone-resorption, an activity to which TNF-αmay contribute, e.g., Bertolini et al., Nature 319: 516-518 (1986) and Johnson et al., Endocrinology 124(3): 1424-1427 (1989). Blocking TNF-α with monoclonal anti-TNF-α antibodies has been shown to be somewhat beneficial in rheumatoid arthritis (Elliot et al., Int. J. Pharm. 17(2): 141-145 (1995)) and Crohn's disease (von Dullemen et al., Gastroenterology, 109(1): 129-135 (2005)); although toxicities can limit their use in these disease conditions.

In chronic, non-productive inflammation, activated monocytes and neutrophils typically play a role in mediating inflammation associated pathology in some of the conditions or diseases attributable to this unresolved inflammatory state. Activated neutrophils can increase production of pro-inflammatory cytokines. Neutrophils can be a source of toxic oxygen species whose generation mediates, at least in part, tumor necrosis factor-alpha (TNF-α) secretion by activated macrophages. TNF-αmay be necessary for some of the organ injury and failure that can be seen in sepsis.

The solid state forms of 3β-tetrol or formulations comprising these solid state forms are therefore useful for modulating or reducing the levels or activities of TNF-α, or one or more pro-inflammatory cytokines described herein (e.g., IL-6 or MCP-1) in vitro or in vivo or to increase numbers of Treg cells in vivo.

Formulations

In some embodiments a formulation comprising or prepared from one or more solid state forms of 3β-tetrol is administered parenterally to a subject having or subject to developing a disease or condition associated with acute or chronic non-productive inflammation. Invention compositions or formulation suitable for use in parenteral administration for human or veterinary applications include liquid solutions, suspensions, emulsions, gels, creams, intramammary infusions, intravaginal delivery systems and implants. Formulations or unit dosage forms suitable for use in oral administration include capsules, caplets, sachets, gelcaps and tablets.

The formulations comprise one or more excipients and a 3β-tetrol in a solid state form as described herein. Such formulations may be for oral administration, e.g., tablets, capsules or gelcaps. Such formulations may be for parenteral administration, e.g., intramuscular or subcutaneous injection. Such formulations may be for topical administration, e.g., creams for application to the skin.

Invention compositions and formulations will comprise or be prepared from androst-5-ene-3β,7β,16α,17β-tetrol (i.e. 3β-tetrol) in solid state form and one or more excipients The excipients are components or ingredients of an invention composition or formulation other than other than the active pharmaceutical ingredient (i.e., 3β-tetrol) that has been found acceptable in the sense of being compatible with the other ingredients or components and has been appropriately evaluated for safety and found not overly deleterious to the patient or animal to which the tetrol compound is to be administered. Compatibility and safety criteria to qualify as an excipient does not mean the excipient is devoid of any chemical, biological or pharmacological activity nor does it require complete inertness. Rather, whatever chemical, biological or pharmacological activities an excipient does posses, the level of activities are acceptable in view of the disease or condition being treated.

Formulations and invention compositions for parenteral administration of 3β-tetrol will usually employ a vehicle as a liquid diluent that provides, e.g., a liquid solution for intravenous injection (i.v.) or a liquid solution or suspension for introduction of 3β-tetrol by intramuscular (i.m.), intradermal or subcutaneous (s.c.) injection. The vehicle may be an oil which forms a solution, suspension or emulsion, that is suitable for non-intravenous routes of parenteral administration, or which form a solution, suspension, emulsion, gel or cream that is suitable for non-injection dependent routes of parenteral administration. A dry powder may be packaged with a propellant to permit nasal or pulmonary deliver of 3β-tetrol, usually as a micronized powder.

Formulations and invention compositions of the present invention may also include tonicity-adjusting agents, particularly in injectable parenteral formulations containing or prepared from one or more crystalline forms of 3β-tetrol. Suitable tonicity adjusting agents are for instance sodium chloride, sodium sulfate, dextrose, mannitol and glycerol, typically mannitol or dextrose.

Buffers agents can include for example those derived from acetic, aconitic, citric, glutaric, lactic, maelic, succinic, phosphate and carbonic acids, as known in the art. Example of buffering agents commonly used in parenteral formulations and of their usual concentrations can be found in Pharmaceutical Dosage Form: Parenteral Medications, Volume 1, 2^{nd} Edition, Chapter 5, p. 194, De Luca and Boylan, “Formulation of Small Volume Parenterals” at Table 5 (Commonly used additives in Parenteral Products). Sometimes the buffering agent is phosphate or citrate buffer present in a buffering agent range between about 10-100 mM to provide a suspension or solution at an initial pH in a pH range between about 4-9, typically between about 4-8.

For parenteral dosage forms, an anti-microbial preservative can be used if no other excipient serves this purpose. Suitable preservatives include, e.g., phenol, resorcinol, chlorobutanol, benzylalcohol, alkyl esters of para-hydroxybenzoic acid such as methyl, ethyl, propyl, butyl and hexyl (generically referred to as parabens), benzalkonium chloride and cetylpyridinium chloride. Sometimes the preservative is an edetate such as a pharmaceutically acceptable salt of EDTA, which may also serves as a metal chelator. Typically, an anti-microbial preservative is present in a preservative range between about 0.001% to 1.0% w/v, typically between about 0.1 to 0.4% or more typically about 0.02%.

Unless otherwise stated or implied by context, expressions of percentage of a liquid excipient in a formulation or an invention composition is given by v/v. Thus, 20% PEG 300 means 20% v/v PEG 300 is present in a liquid or suspension formulation or invention composition. Furthermore, a ratio expression of an amount of a solid excipient in a liquid or suspension formulation or invention composition refers to the excipient's weight relative to the weight of 3β-tetrol present in the liquid or suspension to total volume. Excipients in formulations for use in administration to humans may be NF or USP grade.

In preparing any of the formulations or invention compositions or that are disclosed herein and that comprise or are prepared from one or more crystalline forms of 3β-tetrol (and optionally one or more excipients), one may optionally mill, sieve or otherwise granulate crystalline 3β-tetrol or a formulation or invention composition or comprising crystalline particles of 3β-tetrol in order to obtain a desired average particle size.

Milling may occur before or after the crystalline particles of 3β-tetrol are contacted with one or more excipients. For example, one may mill a crystalline form of 3β-tetrol to obtain a mean particle diameter or a (Dv, 0.90) mean volume diameter of about 0.05-200 microns or about 0.5-30 microns (e.g., about 5, about 10, about 15, about 20, about 25, about 30, about 40, about 60, about 80, about 100 or about 120 microns mean volume weighted particle size or average diameter) before contacting the milled 3β-tetrol particles with a liquid or solid excipient(s).

Micronization may be accomplished by mechanical milling, ultrasonic disintegration, microfluidization, melt extrusion, spray drying, spray freeze-drying or precipitation. Micronization techniques are described in Drug Delivery Technology 2006, 6:54-60; Serajuddin, A T M J. Pharm. Sci. 1999, 88:1058-1066 (hereby specifically incorporated by reference into the present application). Micronization methods using mechanical milling include milling by ball mills, pin mills, jet mills (e.g., fluid energy jet mills). Other methods for micronization include grinding, sieving and precipitation of a compound(s) from a solution, (see, e.g., U.S. Pat. Nos. 4,919,341, 5,202,129, 5,271,944, 5,424,077 and 5,455,049, which are incorporated by reference herein). Particle size is determined by, e.g., transmission electron microscopy, scanning electron microscopy, light microscopy, X-ray diffractometry and light scattering methods or Coulter counter analysis (see, for example, “Characterization of Bulk Solids” D. McGlinchey, Ed., Blackwell Publishing, 2005) The 3β-tetrol crystals can be micronized separately or co-micronized with a surface-active agent, wetting agent or other carrier.

Since grinding, milling, micronization or other mechanical manipulations may result induce a change in polymorph form due to the energy imparted, the particles after such manipulations should be re-evaluated for polymorphism. Sometimes if the mechanical process is sufficiently intensive, the diminished long range ordering and increase presence of crystal defects may occur to an extent that crystalline 3β-tetrol appears present in non-crystalline rather than in a crystalline solid sate form when analyzed by standard XRPD analysis. For example, at average particle sizes lower than 50 angstroms (i.e., 0.005 μm), line widths in XRPD spectra will usually increase beyond 2 2θ (see Jenkins and Snyder “Introduction to X-ray powder diffractometry” Chemical Analysis Series Vol. 38, Wiley-Interscience, 1996). To more readily determine the crystalline form of 3β-tetrol by XRPD after micronization or other such mechanical manipulations, atomic pair distribution functions as described in WO 2005/082050 may be used.

Particle size, unless otherwise specified, refers to a number weighted mean diameter. Sometimes the particle size will be associated with a volume-weighted distribution known as a mean volume diameter and thus the particle size will be the diameter of particles, within a stated fraction (Dv) in a volume-weighted distribution of particles that will have the stated diameter. For example, a particle diameter represented by 35 μm (Dv, 0.90) means that 90% or more of the mass of particles will have a diameter of 35 μm or less. Particle size is determined by, e.g., transmission electron microscopy, scanning electron microscopy, light microscopy, X-ray diffractometry and light scattering methods or Coulter counter analysis (see, for example, “Characterization of Bulk Solids” D. McGlinchey, Ed., Blackwell Publishing, 2005).

For administration of an aqueous-based parenteral dosage form, a sterilized drug product is usually required. A solution dosage form may be sterilized by passage through a microbe-retaining filter or by heat sterilization whereas a suspension dosage from requires sterilization by input of energy. Alternatively, one or more crystalline forms of 3β-tetrol in a blend of solid excipients may be sterilized by ionizing radiation (“cold sterilization” method) and a sterile liquid diluent or a blend of excipients dissolved in the diluent is then added to the solids so sterilized under sterile conditions. Typically, conditions employed for cold sterilization reach 25-30 kGy. Sterilization procedures are discussed in FDA guidance to industry “Sterile drug products produced by aseptic processing” accessible at http://www.fda.gov/cber/gdlns/steraseptic.pdf.

Dosage Forms

Typically, unit doses contain 0.5-500 mg, and more often about 1 mg to about 200 mg, of androst-5-ene-3β,7β,16α,17β-tetrol (i.e., 3β-tetrol). Unit dosage forms include those suitable for oral or parenteral dosing. Preferred unit dosage forms for oral dosing are tablets, capsules, caplets, gel caps and the like.

To limit the amount of water that reaches formulations or invention compositions containing humidity sensitive crystalline forms of 3β-tetrol (e.g., anhydrate crystalline forms including Form VIIβ, Form VIIIβ, Form IXβ and Form Xβ) the formulations and invention compositions may be packaged in hermetically or induction sealed containers. Water permeation characteristics of such containers have been described, e.g., in Containers—Permeation, USP Chp. 23, 1787 et seq., United States Pharmacopeial Convention, Inc., 12601 Twinbrook Parkway, Rockville, Md. 20852, 1995.

Some embodiments of treating a subject using the invention composition or formulation described herein further include monitoring the subject's response to a particular dosing regimen or schedule, e.g., to any continuous or other administration method disclosed herein. For example, while dosing a subject who has an inflammation-based or inflammation-driven diseases or condition one can measure the subject's response, e.g., amelioration of one or more symptoms such as pain or fever or a change in a pro-inflammatory cytokine level. Once a response is observed dosing can be continued for one, two or three additional days, followed by discontinuing the dosing for at least one day (at least 24 hours). Once the subject's response shows signs of recurrence (e.g., a symptom begins to intensify or pro-inflammatory mediator level(s) begins to increase), dosing can be resumed for another course. An aspect of the subject's response to a formulation or invention composition containing 3β-tetrol is that the subject may show a measurable response within a short time, usually about 5-10 days, which allows straightforward tracking of the subject's response, e.g., by monitoring a symptom or a disease biomarker or expression of a pro-inflammatory cytokine or interleukin by e.g., white blood cells or a subset(s) thereof.

For any of the treatments or methods described herein, prolonged beneficial effects or a sustained anti-inflammatory response by a subject may result from a single administration or a few daily administrations of a formulation comprising or prepared from one or more crystalline forms of 3β-tetrol or from intermittent treatment with the 3β-tetrol formulation.

In some embodiments, invention compositions or formulations comprising or prepared from one or more crystalline forms of 3β-tetrol may be used to treat, prevent or slow the progression of or ameliorate one or more conditions in a subject having or subject to developing a chronic, nonproductive inflammation condition wherein the inflammation condition is associated with a metabolic disorder or cardiovascular condition, e.g., hyperglycemia, diabetes or atherosclerosis.

In some embodiments, formulations or invention compositions comprising or prepared from one or more crystalline forms of 3β-tetrol may be used to treat, prevent or slow the progression of or ameliorate one or more conditions in a subject having or subject to developing a inflammatory lung condition, e.g., asthma, chronic bronchitis, COPD, acute respiratory distress syndrome or emphysema.

In some embodiments, formulations or invention compositions comprising or prepared from one or more crystalline forms of 3β-tetrol may be used to treat, prevent or slow the progression of or ameliorate one or more conditions in a subject having or subject to developing a autoimmune disease, e.g., ulcerative colitis, Crohn's disease, multiple sclerosis or arthritis.

In some embodiments, formulations or invention compositions comprising or prepared from one or more crystalline forms of 3β-tetrol may be used to treat, prevent or slow the progression of or ameliorate one or more conditions in a subject having or subject to developing a neurodegenerative disease associated with neuroinflammation, e.g., Alzheimer's disease, amyotrophic lateral sclerosis or Parkinson's disease.

X-ray Powder Diffraction Analysis (XRPD)

XRPD is typically used to characterize or identify crystal compositions (see, e.g., U.S. Pharmacopoeia, volume 23, 1995, method 941, pp. 1843-1845, U.S.P. Pharmacopeia Convention, Inc., Rockville, Md.; Stout et al, X-Ray Structure Determination; A Practical Guide, MacMillan Co., New York, N.Y. 1968). The diffraction pattern obtained from a crystalline compound is often diagnostic for a given crystal form, although weak or very weak diffraction peaks may not always appear in replicate diffraction patterns obtained from successive batches of crystals. This is particularly the case if other crystal forms are present in the sample in appreciable amounts, e.g., when a polymorph of a crystal has become partially hydrated, dehydrated, desolvated or heated to give a significant amount of another crystalline form.

The relative intensities of bands, particularly at low angle X-ray incidence values (low 2θ), may vary due to preferred orientation effects arising from differences in, e.g., crystal habit, particle size and other conditions of measurement. Individual XRPD peaks in different samples are generally located within about 0.3±1 2θ degree for broad peaks. Broad XRPD peaks may sometimes appear as two or more individual peaks located closely together. For sharp isolated peaks under reproducible conditions, the peak is usually found within about 0.2 2θ degrees on successive XRPD analyses. Thus, when a sharp isolated XRPD peak at a given position is identified as being located at, e.g., about 16.1, this means that the peak is at 16.1±0.1. It is usually not necessary to rely on all bands that one observes for a given crystalline form disclosed herein; sometimes even a single band may be diagnostic for a given polymorphic form 3β-tetrol.

Typically, individual crystalline forms of 3β-tetrol are characterized by reference to 2, 3 or 4 XRPD having the most intensity or the 2, 3 or 4 most reproducible peaks XRPD peaks and optionally by reference to one or two other physical or analytical properties such as melting point, one or more thermal transitions observed in DTA and/or differential scanning calorimetry (DSC), one or more absorption peaks observed in infrared spectroscopy (IR) and/or dissolution rate (DR) data in an aqueous or other solvent system. Standardized methods for obtaining XRPD, DTA, DSC, DR, etc. data have been described (see U.S. Pharmacopoeia, volume 23, 1995, United States Pharmacopeial Convention, Inc., Rockville, Md., pp 2292-2296 and 2359-2765).

Prominent XRPD peaks are preferably selected from observed peaks by identifying non-overlapping, low-angle peaks. A prominent peak will have relative intensity of at least about 5% or more typically at least about 10% or at least about 15% or at least about 20% relative intensity in comparison to the most intense peak in the X-ray diffraction pattern. Sometimes one or more peaks of intensity lower than 5% may be considered prominent and are used in addition with one or more peaks that are more prominent (i.e. at least about 10% or at least about 15% or at least about 20% relative intensity) in order to describe an XRPD pattern for a crystalline form of 3β-tetrol.

For identifying a crystalline form of 3β-tetrol in a solid formulation or invention composition, such as a tablet or in capsule granules, pairwise distribution function plots of principal components as described in US Pat. Pub. No. 2007/0110214 (which is incorporated by reference herein) are linearly combined and compared with the pairwise distribution plot of the solid formulation or invention composition. If the crystalline tetrol compound has been ground or micronized to such an extent that excessive line broadening prevents acquisition of meaningful XRPD data, precession electron diffraction using transmission electron microscopy as described in US Pat. Appl. No. 2007/0023659 (which is incorporated by reference herein) may be used as an alternative diffraction technique for identification of the crystalline form in such solid mixtures.

Vibrational Spectroscopy

Diagnostic techniques that one can optionally use to characterize crystalline forms of 3β-tetrol include vibrational spectroscopy techniques such as IR and Raman, which measure the effect of incident energy on a solid state sample due to the presence of particular chemical bonds within molecules of the sample that vibrate in response to the incident energy. Since the molecules in different polymorphs experience different intermolecular forces due to variations in conformational or environmental factors, perturbations of those vibrations occur that leads to differences in spectra due to differences in frequency and intensity of some modes of vibration. Because polymorphs may possess different IR and Raman characteristics, IR and Raman spectrum provide complementary information and either may provide a fingerprint for identification of a particular polymorph. [see, Anderton, C., Eur. Pharm. Rev., 9:68-74 (2004)].

In contrast to IR spectroscopy, Raman spectroscopy relies upon measuring light scattered from incident radiation of a particular wavelength directed to the sample, which can range from the UV to the near-IR. The light scattered contains not only photons with the same frequency as that of the incident radiation (called Rayleigh scattered light, which is filtered out), but also photons with a shifted frequency due to inelastic collisions with molecules within the solid state sample and it is these shifted frequencies that are determined by Raman spectroscopy. Thus, Raman scattered light is frequency-shifted (Raman-shift) with respect to the excitation frequency, but the magnitude of the shift is independent of the excitation frequency. Because Raman scattered light changes in frequency, the rule of conservation of energy dictates that some energy is deposited in the sample. Thus, a Raman shift will correspond to the excitation energy of a particular free vibration of a molecule in the solid state sample and is therefore an intrinsic property of the sample. While some molecular vibrations may be observed in both the IR and Raman spectra, sometimes vibrations observed using one technique will be weak or completely absent in the other due to the different fundamental physics underlying the two techniques.

Sample preparation in Raman spectroscopy is minimal, and If sample is limited it may be dispersed in oil or mixed with KBr to give enough material for introduction into the Raman spectrophotometer. Raman is also capable of determining polymorph identity or quantification in a complex matrix, distinguishing between non-crystalline and crystalline forms and is capable of differentiating between multiple polymorphic and pseudo polymorphic forms [for example, see Pratiwia, D., et al. “Quantitative analysis of polymorphic mixtures of ranitidine hydrochloride by Raman spectroscopy and principal components analysis” Eur. J. Pharm. Biopharm. 54(3), 337-341 (2002)]. These capabilities are possible because features in the Raman are sharp and well resolved, which limits interference from excipients, and are very sensitive to the polymorphic form of a compound, with substantial spectral differences often evident between polymorphs, which also facilitates identification of more subtle features that may indicate the presence of a low level of one polymorph within another. For identifying a polymorph in a solid formulation such as a tablet, powder samples of pure 3β-tetrol polymorphs or pseudo-polymorphs and excipients are gently compacted and scanned with Raman microscopy to build up a library of formulation component spectra. A partial least squares (PLS) model and multivariate classification are then used to analyze Raman mapping data obtained from sectioned tablets having low API content (about 0.5% w/w). Multivariate classification allows polymorph assignments to be made on individual microscopic pixels of 3β-tetrol identified in the data. By testing data from separate sets of tablets containing each polymorph, specific form recognition may be demonstrated at about 0.5% w/w. For tablets containing a mixture of forms, recognition of about 10% polymorphic impurity of 3β-tetrol (representing an absolute detection limit of about 0.05% w/w), is possible.

Overlap of IR bands from different solid state forms using the various methods described above sometimes occurs so that quantification requires deconvolution methods to extract information for each individual component in a mixture of crystalline forms. Such methods include partial least squares regression, principle component analysis or other methodologies [for examples, see Reich, G. “Near-infrared spectroscopy and imaging: Basic principles and pharmaceutical applications” Adv. Drug Deliv. Rev. 57: 1109-43 (2005)].

In one embodiment a crystalline form of 3β-tetrol is characterized by its XRPD and a spectrum obtained from a vibrational spectroscopy method, with Raman spectroscopy preferred.

Thermo Analysis Procedures

Diagnostic techniques that one can optionally use to characterize a crystalline form of 3β-tetrol include differential thermal analysis (DTA), differential scanning calorimetry (DSC), thermo-gravimetric analysis (TGA) and melting point measurements.

DTA and DSC measures thermal transition temperatures at which a crystalline sample absorbs or releases heat when its crystal structure changes or it melts. TGA is used to measure thermal stability and the fraction of volatile components of a sample by monitoring the weight change as the sample is heated. These techniques are thus useful for characterizing crystalline forms existing as solvates and/or hydrates (i.e., pseudo-polymorphs).

DTA involves heating a test sample and an inert reference under identical conditions while recording any temperature difference between the sample and reference. This differential temperature is plotted against temperature, and changes in the test sample that leads to absorption or liberation of heat can thus be determined relative to the inert sample.

DSC measures the energy needed to establish a nearly zero temperature difference between a sample and an inert reference as they are subjected to identical heating regimes. In power compensated DSC, the temperatures of the sample and reference are controlled independently. The temperature of the sample and reference are made identical by varying the power input of the two heaters in which the sample and reference reside. The energy required to do this is a measure of the enthalpy or heat capacity changes in the sample relative to the reference.

Transition temperatures in DSC or DTA for sharply-defined endotherms or exotherms typically occur within about 4° C. on successive analyses of crystalline 3β-tetrol samples using a temperature scan rate of 10° C./min. Thus, when a crystalline form of 3β-tetrol is reported to have a thermal transition at a given value, it means that the DTA or DSC transition is within ±2° C. of the reported value. Different crystalline forms may be identified, at least in part, based on their different transition temperature profiles in their DTA or DSC thermographs and optionally based upon their weight loss in TGA within a defined temperature range.

Summary of DTA events observed for crystalline forms of 3β-tetrol described herein using a temperature scan rate of 10° C./min is given in Table 1. Thermal traces underlying this data are provided in the Figures where the thermogravimetric trace is the upper line and the DTA trace is the lower line.

TABLE 1
DTA Events for 3β-Tetrol Crystalline Forms

| Crystalline | Solvent | En^{sol} | En^{pol} | Exo^{pol} | Tₒₙ | Tₘ |
|---|---|---|---|---|---|---|
| Form | System | (° C.)^{a} | (° C.)^{b} | (° C.)^{c} | (° C.)^{d} | (° C.)^{e} |
| Iβ | ACN- | 97.6 | | | 194.3 | 204.4 |
| | water | | | | | (major) |
| | | | | | | 224.0 |
| | | | | | | (minor) |
| IIβ | EtOH^{f}- | 102.5 | 238.8 | 242.0 | | 252.4 |
| | ACN | | | | | |
| IIIβ | EtOH^{g}- | 103.5 | | | 191.2 | 199.8 |
| | ACN | | | | | (dec) |
| | | | | | | 210.4 |
| | | | | | | (shld) |
| IVβ | EtOH^{g}- | 99.2 | 196.9 | | 227.5 | 232.8 |
| | heptane | | | | | |
| Vβ | MeOH | 102.3 | | 188.4 | 222.8 | 229.8 |
| VIβ | MeOH | 111.0 | | | 226.1 | 232.5 |
| VIIβ | NA | | | | 245.3 | 251.7 |
| VIIIβ | MeOH | | | 177.6 | 224.6 | 233.1 |
| VIXβ | EtOH^{f}- | | 180.0 | | | 229 |
| | water | | | | | (dec)^{†} |
| | | | 187.5 | | | |
| | | | (shld) | | | |
| Xβ | EtOH^{h}- | | | | 189.8 | 204.0 |
| | EtOAc | | | | | (dec) |
| | | | | | | 216.6 |
| | | | | | | (shld) |

^{a}Endotherm Peak Temperature-desolvation;
^{b}Endotherm Peak Temperature-polymorph Transition;
^{c}Exotherm Peak Temperature-Polymorph Transition;
^{d}Onset Temperature for final melt;
^{e}Temperature(s) of final melt or decomposition;
^{f}90% EtOH reagent grade;
^{g}denatured EtOH;
^{h}absolute EtOH;
^{†}very broad endotherm,
NA = not applicable,
shld = shoulder;
dec = decomposition

### Numbered Embodiments

The following embodiments exemplify and/or describe one or more aspects of the invention are not meant to be limiting in any way.

1. Crystalline androst-5-ene-3β,7β,16α,17β-tetrol provided that crystalline androst-5-ene-3β,7β,16α,17β-tetrol is not Form Iβ 3β-tetrol.

2. The crystalline androst-5-ene-3β,7β,16α,17β-tetrol of embodiment 1 wherein crystalline androst-5-ene-3β,7β,16α,17β-tetrol is Form IIβ, Form IIIβ, Form IVβ, Form Vβ, Form VIβ, Form VIIβ, Form VIIIβ, Form IXβ or Form Xβ 3β-tetrol.

3. The crystalline androst-5-ene-3β,7β,16α,17β-tetrol of embodiment 1 wherein crystalline androst-5-ene-3β,7β,16α,17β-tetrol is Form IIβ 3β-tetrol characterized by DTA thermogram, obtained with a temperature ramp of 10° C./min, having a prominent endotherm centered at about 252° C., an endotherm centered at about 239° C., optionally with an onset temperature of about 235° C., and an exotherm centered at about 242° C.

4. The crystalline androst-5-ene-3β,7β,16α,17β-tetrol of embodiment 3 wherein crystalline androst-5-ene-3β,7β,16α,17β-tetrol is further characterized a TGA thermogram, obtained with a temperature ramp of 10° C./min, having about 5% wt loss from between about 60° C. to about 140° C., optionally associated with a broad endotherm in DTA centered at about 102° C.

5. The crystalline androst-5-ene-3β,7β,16α,17β-tetrol of embodiment 1 wherein crystalline androst-5-ene-3β,7β,16α,17β-tetrol is Form IIIβ 3β-tetrol characterized by (1) XRPD pattern having three or more peaks selected from the group consisting of about 7.6, 14.9, 25.4 and 29.6 degree 2-theta and optionally one or more peaks selected from the group consisting of about 15.4, 16.1, 17.3 and 19.9 degree 2-theta or (2) DTA thermogram, obtained with a temperature ramp of 10° C./min, having a prominent endotherm centered at about 200° C., optionally having an onset temperature of about 191° C. and/or a shoulder at about 210° C., or (3): (1) and (2).

Form IIIβ 3β-tetrol can be characterized by an XRPD peak at about 7.6, 14.9, 25.4 and 29.6 degree 2-theta and a DTA thermogram, obtained with a temperature ramp of 10° C./min, having a prominent endotherm centered at about 200° C., optionally having an onset temperature of about 191° C. and/or a shoulder at about 210° C. and one XRPD peak at about 15.4, 16.1, 17.3 or 19.9 degree 2-theta.

Form IIIβ 3β-tetrol can also be characterized by an XRPD peak at about 7.6, 14.9, 25.4 and 29.6 degree 2-theta and a DTA thermogram, obtained with a temperature ramp of 10° C./min, having a prominent endotherm centered at about 200° C., optionally having an onset temperature of about 191° C. and/or a shoulder at about 210° C. and two XRPD peaks at about 15.4 and 16.1, about 15.4 and 17.3, 15.4 and 19.9, 16.1 and 17.3, about 16.1 and 19.9 or 17.3 and 19.9 degree 2-theta.

Form IIIβ 3β-tetrol can also be characterized by an XRPD peak at about 7.6, 14.9, 25.4 and 29.6 degree 2-theta and a DTA thermogram, obtained with a temperature ramp of 10° C./min, having a prominent endotherm centered at about 200° C., optionally having an onset temperature of about 191° C. and/or a shoulder at about 210° C. and three XRPD peaks at about 15.4, 16.1 and 17.3, about 15.4, 16.1 and 19.9 or about 16.1, 17.3 and 19.9 degree 2-theta.

Form IIIβ 3β-tetrol can also be characterized by an XRPD peak at about 7.6, 14.9, 25.4 and 29.6 degree 2-theta and (a) three XRPD peaks at about 15.4, 16.1 and 17.3, about 15.4, 16.1 and 19.9 or about 16.1, 17.3 and 19.9 degree 2-theta (b) two XRPD peaks at about 15.4 and 16.1, about 15.4 and 17.3, 15.4 and 19.9, 16.1 and 17.3, about 16.1 and 19.9 or 17.3 and 19.9 degree 2-theta or (c) four XRPD peaks at about 15.4, 16.1, 17.3 and 19.9 degree 2-theta.

6. The crystalline androst-5-ene-3β,7β,16α,17β-tetrol of embodiment 5 wherein crystalline androst-5-ene-3β,7β,16α,17β-tetrol is further characterized by (1) TGA thermogram, obtained with a temperature ramp of 10° C./min, having about 5% wt loss from between about 60° C. to about 140° C., optionally associated with a broad endotherm in DTA thermogram centered at about 103° C., and between about 5% to about 10% wt loss or more associated with the 200° C. DTA endotherm or (2) solid state Raman spectrum having absorbances at about 1275, 1329, 1344 and 1437 cm⁻¹ or four or more absorbances selected from the group consisting of about 445, 474, 987, 1057, 1091 and 1128 cm⁻¹ or (3): (1) and (2).

7. The crystalline androst-5-ene-3β,7β,16α,17β-tetrol of embodiment 1 wherein crystalline androst-5-ene-3β,7β,16α,17β-tetrol is Form IVβ 3β-tetrol characterized by (1) XRPD pattern having three or more peaks selected from the group consisting of about 7.7, 15.4, 16.2 and 25.3 degree 2-theta and one or more peaks selected from the group consisting of about 14.8, 19.7, 20.8 and 29.9 degree 2-theta or (2) DTA thermogram, obtained with a temperature ramp of 10° C./min, having a prominent endotherm centered at about 233° C., optionally having an onset temperature of about 228° C., and a weak, broad endotherm centered at about 197° C., optionally with an onset temperature of about 189° C., or (3): (1) and (2).

Form IVβ 3β-tetrol can be characterized by an XRPD pattern having peaks at about 7.7, 15.4, 16.2 and 25.3 degree 2-theta and one XRPD peak at about 14.8, 19.7, 20.8 or 29.9 degree 2-theta and optionally a DTA thermogram, obtained with a temperature ramp of 10° C./min, having a prominent endotherm centered at about 233° C., with an onset temperature of about 228° C. and an endotherm centered at about 197° C. with an onset temperature of about 189° C.

Form IVβ 3β-tetrol can also be characterized by an XRPD pattern having peaks at about 7.7, 15.4, 16.2 and 25.3 degree 2-theta and two XRPD peaks at about 14.8 and 19.7, about 14.8 and 20.8, about 14.8 and 29.9, about 19.7 and 20.9, about 19.7 and 29.9 or about 20.9 and 29.9 degree 2-theta and optionally a DTA thermogram, obtained with a temperature ramp of 10° C./min, having a prominent endotherm centered at about 233° C., with an onset temperature of about 228° C. and an endotherm centered at about 197° C. with an onset temperature of about 189° C.

Form IVβ 3β-tetrol can also be characterized by an XRPD pattern having peaks at about 7.7, 15.4, 16.2 and 25.3 degree 2-theta and three XRPD peaks at about 14.8, 19.7 and 20.8, about 14.8, 19.7 and 29.9 or about 19.7, 20.9 and 29.9 degree 2-theta and optionally a DTA thermogram, obtained with a temperature ramp of 10° C./min, having a prominent endotherm centered at about 233° C., with an onset temperature of about 228° C. and an endotherm centered at about 197° C. with an onset temperature of about 189° C.

8. The crystalline androst-5-ene-3β,7β,16α,17β-tetrol of embodiment 7 wherein crystalline androst-5-ene-3β,7β,16α,17β-tetrol is further characterized by (1) TGA thermogram, obtained with a temperature ramp of 10° C./min, having about 5% wt loss from between about 60° C. to about 140° C., optionally associated with a broad endotherm in DTA centered at about 99° C. or (2) solid state Raman spectrum having absorbances at about 1279, 1329, 1342 and 1437 cm⁻¹ or four or more absorbances selected from the group consisting of about 443, 474, 517, 536, 901, 985, 1009, 1045, 1090, 1099 and 1172 cm⁻¹ or (3): (1) and (2).

9. The crystalline androst-5-ene-3β,7β,16α,17β-tetrol of embodiment 1 wherein crystalline androst-5-ene-3β,7β,16α,17β-tetrol is Form Vβ 3β-tetrol characterized by (1) XRPD pattern having three or more peaks selected from the group consisting of about 7.4, 14.8, 15.9, 17.3, 19.2, 20.2, 24.4 and 29.4 degree 2-theta and one or more peaks selected from the group consisting of about 14.6, 17.8, 19.8, 20.3, 21.1, 22.7, 25.5 and 27.3 degree 2-theta or (2) DTA thermogram, obtained with a temperature ramp of 10° C./min, having a prominent endotherm centered at about 230° C., optionally having an onset temperature of about 223° C., and a weak exotherm centered at about 188° C. or (3): (1) and (2).

Form Vβ 3β-tetrol can be characterized by (a) an XRPD pattern having four peaks at about 7.4, 14.8, 15.9 and 17.3, or about 7.4, 14.8, 15.9 and 19.2, or about 7.4, 14.8, 15.9 and 20.2, or about 7.4, 14.8, 15.9 and 24.4 or about 7.4, 14.8, 15.9 and 29.4 degree 2-theta, (b) one XRPD peak at about 14.6, 17.8, 19.8, 20.3, 21.1, 22.7, 25.5 or 27.3 degree 2-theta and (c) optionally (2).

Form Vβ 3β-tetrol can also be characterized by (a) an XRPD pattern having peaks at about 14.8, 15.9, 17.3 and 19.2, or about 14.8, 15.9, 17.3 and 20.2, or about 14.8, 15.9, 17.3 and 24.4, or about 14.8, 15.9, 17.3 and 29.4 degree 2-theta (b) one XRPD peak at about 14.6, 17.8, 19.8, 20.3, 21.1, 22.7, 25.5 or 27.3 degree 2-theta and optionally (2).

Form Vβ 3β-tetrol can also be characterized by (a) an XRPD pattern having peaks at about 15.9, 17.3, 19.2 and 20.2, or about 15.9, 17.3, 19.2 and 24.4, or about 15.9, 17.3, 19.2 and 29.4 degree 2-theta (b) one XRPD peak at about 14.6, 17.8, 19.8, 20.3, 21.1, 22.7, 25.5 or 27.3 degree 2-theta and optionally (2).

Form Vβ 3β-tetrol can also be characterized by (a) an XRPD pattern having peaks at about 17.3, 19.2, 20.2 and 24.4, or about 17.3, 19.2, 20.2 and 29.4, or about 17.3, 19.2, 24.4 and 29.4 degree 2-theta (b) one XRPD peak at about 14.6, 17.8, 19.8, 20.3, 21.1, 22.7, 25.5 or 27.3 degree 2-theta and optionally (2).

Form Vβ 3β-tetrol can be characterized by (a) an XRPD pattern having five peaks at about 7.4, 14.8, 15.9, 17.3 and 19.2, about 7.4, 14.8, 15.9, 17.3 and 20.2, about 7.4, 14.8, 15.9, 17.3 and 24.4 or about 7.4, 14.8, 15.9, 17.3 and 29.4 degree 2-theta, (b) one XRPD peak at about 14.6, 17.8, 19.8, 20.3, 21.1, 22.7, 25.5 or 27.3 degree 2-theta and (c) optionally (2).

Form Vβ 3β-tetrol can be characterized by (a) an XRPD pattern having five peaks at about 14.8, 15.9, 17.3, 19.2 and 20.2, about 14.8, 15.9, 17.3, 19.2 and 24.4, or about 14.8, 15.9, 17.3, 19.2 and 29.4 degree 2-theta, (b) one XRPD peak at about 14.6, 17.8, 19.8, 20.3, 21.1, 22.7, 25.5 or 27.3 degree 2-theta and (c) optionally (2).

Form Vβ 3β-tetrol can also be characterized by (a) an XRPD pattern having five peaks at about 15.9, 17.3, 19.2, 20.2 and 24.4, about 15.9, 17.3, 19.2, 20.2 and 29.4 or about 17.3, 19.2, 20.2, 24.4 and 29.4 degree 2-theta, (b) one XRPD peak at about 14.6, 17.8, 19.8, 20.3, 21.1, 22.7, 25.5 or 27.3 degree 2-theta and (c) optionally (2).

10. The crystalline androst-5-ene-3β,7β,16α,17β-tetrol of embodiment 9 wherein crystalline androst-5-ene-3β,7β,16α,17β-tetrol is further characterized by (1) TGA thermogram, obtained with a temperature ramp of 10° C./min, having about 5% wt loss from between about 60° C. to about 140° C., optionally associated with a broad endotherm in DTA centered at about 102° C. or (2) solid state Raman spectrum having absorbances at about 1279, 1329, 1344 and 1439 cm⁻¹ or four or more absorbances selected from the group consisting of about 443, 472, 536, 985, 1009, 1055, 1099 and 1172 cm⁻¹ or (3): (1) and (2).

11. The crystalline androst-5-ene-3β,7β,16α,17β-tetrol of embodiment 1 wherein crystalline androst-5-ene-3β,7β,16α,17β-tetrol is Form VIβ 3β-tetrol characterized by (1) XRPD pattern having three or more peaks selected from the group consisting about 6.5, 7.7, 8.2, 13.1, 15.0, 15.4, 16.2, 17.0, 19.9, 22.3 and 25.4 degree 2-theta and one or more peaks selected from the group consisting of about 9.7, 14.8, 15.9, 16.7, 19.3, 19.6, 20.8, 20.9, 21.1, 21.2, 21.9, 23.1, 23.5, 23.9, 24.6, 25.3 and 29.8 degree 2-theta or (2) DTA thermogram, obtained with a temperature ramp of 10° C./min, having a prominent endotherm centered at about 233° C., optionally having an onset temperature of about 226° C., and no endotherm between about 140° C. to about 200° C. or (3) solid state Raman spectrum having four or more absorbances selected from the group consisting of about 1196, 1232, 1250, 1273, 1319, 1344, 1439 and 1462 cm⁻¹ or four or more absorbances selected from the group consisting of about 440, 447, 476, 519, 696, 901, 987, 1007, 1036, 1059 and 1091 cm⁻¹ or (4): (1) and (2), (1) and (3), (2) and (3) or (1), (2) and (3).

12. The crystalline androst-5-ene-3β,7β,16α,17β-tetrol of embodiment 11 wherein crystalline androst-5-ene-3β,7β,16α,17β-tetrol is further characterized by TGA thermogram, obtained with a temperature ramp of 10° C./min, having about 10% wt loss from between about 60° C. to about 140° C., optionally associated with a broad endotherm in DTA centered at about 111° C.

13. The crystalline androst-5-ene-3β,7β,16α,17β-tetrol of embodiment 1 wherein crystalline androst-5-ene-3β,7β,16α,17β-tetrol is Form VIIβ 3β-tetrol characterized by DTA thermogram, obtained with a temperature ramp of 10° C./min, having a prominent endotherm centered at about 252° C., optionally having an onset temperature of about 239° C., and no thermal transitions from between about 60° C. to about the onset temperature of the prominent endotherm.

14. The crystalline androst-5-ene-3β,7β,16α,17β-tetrol of embodiment 1 wherein crystalline androst-5-ene-3β,7β,16α,17β-tetrol is Form VIIIβ 3β-tetrol characterized by (1) XRPD pattern having three or more peaks selected from the group consisting of about 6.1, 12.2, 16.2 and 25.3 degree 2-theta and one or more peaks selected from the group consisting of about 7.6, 8.1, 15.4, 18.2, 19.6, 19.9, 20.8 and 29.8 degree 2-theta or (2) DTA thermogram, obtained with a temperature ramp of 10° C./min, having a prominent endotherm centered at about 233° C., optionally having an onset temperature of about 225° C., and a broad endotherm centered at about 178° C., optionally having an onset temperature of about 163° C. or (3): (1) and (2).

15. The crystalline androst-5-ene-3β,7β,16α,17β-tetrol of embodiment 14 wherein crystalline androst-5-ene-3β,7β,16α,17β-tetrol is further characterized by (1) TGA thermogram, obtained with a temperature ramp of 10° C./min, having negligible % wt loss from between about 60° C. to about 140° C., optionally associated with a broad endotherm in DTA thermogram centered at about 85° C. of variable intensity or (2) solid state Raman spectrum having absorbances at about 1277, 1329, 1346 and 1439 cm⁻¹ or four or more absorbances selected from the group consisting of about 443, 474, 987, 1010, 1057 and 1099 cm⁻¹ or (3): (1) and (2).

16. The crystalline androst-5-ene-3β,7β,16α,17β-tetrol of embodiment 1 wherein crystalline androst-5-ene-3β,7β,16α,17β-tetrol is Form IXβ 3β-tetrol characterized by (1) XRPD pattern having three or more peaks selected from the group consisting of about 7.4, 14.9, 15.9, 17.3, 20.4 and 24.4 degree 2-theta and one or more peaks selected from the group consisting of about 14.6, 17.9, 19.2, 19.8, 20.2, 25.6, 27.4 and 29.4 degree 2-theta or (2) DTA thermogram, obtained with a temperature ramp of 10° C./min, having a prominent endotherm centered at about 180° C., optionally having an onset temperature of about 165° C. or a shoulder at about 187° C., or (3): (1) and (2).

17. The crystalline androst-5-ene-3β,7β,16α,17β-tetrol of embodiment 16 wherein crystalline androst-5-ene-3β,7β,16α,17β-tetrol is further characterized by TGA thermogram, obtained with a temperature ramp of 10° C./min, having (1) negligible % wt loss from between about 60° C. to about 140° C., optionally associated with a broad endotherm in DTA centered at about 81° C. of variable intensity, (2) between about 5% to about 10% wt loss or more associated with the 180° C. DTA endotherm or (3) between about 5% to about 10% wt loss or more associated with a very broad DTA endotherm between about 220° C. to about 260° C. or (4): (1) and (3), (2) and (3) or (1), (2) and (3).

18. The crystalline androst-5-ene-3β,7β,16α,17β-tetrol of embodiment 16 or 17 wherein crystalline androst-5-ene-3β,7β,16α,17β-tetrol is Form IXβ 3β-tetrol is further characterized by solid state Raman spectrum having absorbances at about 1277, 1327, 1346 and 1437 cm⁻¹ or four or more absorbances selected from the group consisting of about 443, 472, 536, 598, 901, 985, 1009, 1057 and 1099 cm⁻¹.

19. The crystalline androst-5-ene-3β,7β,16α,17β-tetrol of embodiment 1 wherein crystalline androst-5-ene-3β,7β,16α,17β-tetrol is Form Xβ 3β-tetrol characterized by (1) XRPD pattern having three or more XRPD peaks selected from the group consisting of about 6.1, 12.1, 13.0, 13.6, 14.0, 15.8, 18.2 and 18.6 degree 2-theta and one or more peaks selected from the group consisting of about 8.1, 9.9, 16.8, 19.8, 20.8, 21.8, 24.3 and 29.8 degree 2-theta or (2) DTA thermogram, obtained with a temperature ramp of 10° C./min, having a prominent endotherm centered at about 204° C., optionally having an onset temperature of about 190° C. and/or a shoulder at about 217° C., or (3): (1) and (2).

20. The crystalline androst-5-ene-3β,7β,16α,17β-tetrol of embodiment 19 wherein crystalline androst-5-ene-3β,7β,16α,17β-tetrol is further characterized by TGA thermogram, obtained with a temperature ramp of 10° C./min, having negligible % wt loss from between about 60° C. to about 140° C., optionally associated with a broad endotherm in DTA centered at about 81° C. of variable intensity and between about 5-10% wt loss or more associated with the DTA 204° C. endotherm.

21. A crystalline hydrate of androst-5-ene-3β,7β,16α,17β-tetrol provided that the crystalline hydrate is not Form Iβ 3β-tetrol.

22. The crystalline hydrate of androst-5-ene-3β,7β,16α,17β-tetrol wherein the crystalline hydrate is a monohydrate or a dihydrate

23. The crystalline hydrate of embodiment 21 wherein the hydrate is Form IIβ, Form IIIβ, Form IVβ, Form Vβ or Form VIβ 3β-tetrol.

24. A crystalline anhydrate of androst-5-ene-3β,7β,16α,17β-tetrol.

25. The crystalline anhydrate of embodiment 24 where the anhydrate is Form VIIβ, Form VIIIβ, Form IXβ or Form Xβ 3β-tetrol.

26. A composition comprising, consisting essentially of or consisting of one or more excipients and a solid state form of androst-5-ene-3β,7β,16α,17β-tetrol provided the solid state form is not Form Iβ 3β-tetrol.

27. The composition of embodiment 26 wherein the solid state form is crystalline androst-5-ene-3β,7β,16α,17β-tetrol.

28. The composition of embodiment 26 wherein the solid state form of androst-5-ene-3β,7β,16α,17β-tetrol is a crystalline hydrate.

29. The composition of embodiment 28 wherein the crystalline hydrate is a monohydrate or a dihydrate

30. The composition of embodiment 28 wherein the crystalline hydrate is Form IIβ, Form IIIβ, Form IVβ, Form Vβ or Form VIβ 3β-tetrol.

31. The composition of embodiment 26 wherein the solid state form is a crystalline anhydrate.

32. The composition of embodiment 29 wherein the crystalline anhydrate is Form VIIβ, Form VIIIβ, Form IXβ or Form Xβ 3βtetrol.

33. A method of preparing a liquid formulation comprising, consisting essentially of or consisting of admixing a solid state form of androst-5-ene-3β,7β,16α,17β-tetrol with a liquid excipient provided the solid state form is not Form Iβ 3β-tetrol.

34. The method of embodiment 33 wherein the solid state form is crystalline androst-5-ene-3β,7β,16α,17β-tetrol

35. The method of embodiment 34 hydrate the crystalline androst-5-ene-3β,7β,16α,17β-tetrol is a crystalline hydrate.

36. The method of embodiment 35 wherein the crystalline hydrate is a monohydrate or a dihydrate.

37. The method of embodiment 35 wherein the crystalline hydrate is Form IIβ, Form IIIβForm IVβ, Form Vβ or Form VIβ 3β-tetrol.

38. The method of embodiment 33 wherein the solid state form is a crystalline anhydrate.

39. The method of embodiment 38 wherein the crystalline anhydrate is Form VIIβ, Form VIIIβ, Form IXβ or Form Xβ 3β-tetrol.

40. A method of treating unwanted inflammation, comprising administering an effective amount of a solid formulation to a subject in need thereof wherein the solid formulation comprises, consists essentially of or consists of one or more excipients and a solid state form of androst-5-ene-3β,7β,16α,17β-tetrol provided the solid state form is not Form Iβ 3β-tetrol.

41. The method of embodiment 37 wherein the solid state form is crystalline androst-5-ene-3β,7β,16α,17β-tetrol.

43. The method of embodiment 37 wherein the solid state form of androst-5-ene-3β,7β,16α,17β-tetrol is a crystalline hydrate.

44. The method of embodiment 43 wherein the crystalline hydrate is a monohydrate or a dihydrate.

45. The method of embodiment 43 wherein the crystalline hydrate is Form IIβ, Form IIIβForm IVβ, Form Vβ or Form VIβ 3β-tetrol.

46. The method of embodiment 40 wherein the solid state form is a crystalline anhydrate.

47. The method of embodiment 46 wherein the crystalline anhydrate is Form VIIβ, Form VIIIβ, Form IXβ or Form Xβ 3β-tetrol.

48. The method of embodiment 40 wherein the unwanted inflammation is a condition or disease associated with chronic, non-production inflammation.

49. The method of embodiment 48 wherein the condition or disease is an autoimmune condition or disease.

50. The method of embodiment 48 wherein the condition or disease is a metabolic condition or disease.

51. The method of embodiment 49 wherein the autoimmune disease is Type 1 diabetes.

52. The method of embodiment 49 wherein the autoimmune disease is a lupus condition such as systemic lupus erythematosus or discoid lupus or an arthritis condition such as rheumatoid arthritis.

53. The method of embodiment 48 wherein the condition or disease is an inflammatory bowel disease such as ulcerative colitis or Crohn's disease (regional enteritis).

54. The method of embodiment 49 wherein the condition or disease is a lung inflammation condition such as cystic fibrosis, chronic obstructive pulmonary disease (COPD), acute respiratory disease syndrome, acute asthma, chronic asthma, emphysema, acute bronchitis, allergic bronchitis, chronic bronchitis and fibrosing alveolitis (lung fibrosis) conditions, e.g., subepithelial fibrosis in patients having chronic bronchitis, asthma and/or COPD.

55. The method of embodiment 49 wherein the condition or disease is a neurodegenerative condition such as Parkinson's disease or Alzheimer's disease.

56. The method of embodiment 49 wherein the condition or disease is a hyperproliferation condition.

57. The method of embodiment 49 wherein the condition or disease is a liver cirrhosis condition, nonalcoholic steatohepatitis (NASH) or nonalcoholic fatty liver disease.

58. The method of embodiment 50 wherein the metabolic condition or disease is type 2 diabetes, obesity, insulin resistance, hyperglycemia, impaired glucose utilization or tolerance, impaired or reduced insulin synthesis. In preferred embodiments, the metabolic condition is hyperglycemia or type 2 diabetes.

59. The crystalline androst-5-ene-3β,7β,16α,17β-tetrol of embodiment 1 or 2, the composition of embodiment 26 or the method of any one of embodiments 40, 42 or 48-58 wherein the solid state form or crystalline form of androst-5-ene-3β,7β,16α,17β-tetrol is Form IIβ 3β-tetrol.

60. The crystalline androst-5-ene-3β,7β,16α,17β-tetrol of embodiment 1 or 2, the composition of embodiment 26 or the method of any one of embodiments 40, 42 or 48-58 wherein the solid state form or crystalline form of androst-5-ene-3β,7β,16α,17β-tetrol is Form IIIβ 3β-tetrol.

61. The crystalline androst-5-ene-3β,7β,16α,17β-tetrol of embodiment 1 or 2, the composition of embodiment 26 or the method of any one of embodiments 40, 42 or 48-58 wherein the solid state form or crystalline form of androst-5-ene-3β,7β,16α,17β-tetrol is Form IVβ 3β-tetrol.

62. The crystalline androst-5-ene-3β,7β,16α,17β-tetrol of embodiment 1 or 2, the composition of embodiment 26 or the method of any one of embodiments 40, 42 or 48-58 wherein the solid state form or crystalline form of androst-5-ene-3β,7β,16α,17β-tetrol is Form Vβ 3β-tetrol.

63. The crystalline androst-5-ene-3β,7β,16α,17β-tetrol of embodiment 1 or 2, the composition of embodiment 26 or the method of any one of embodiments 40, 42 or 48-58 wherein the solid state form or crystalline form of androst-5-ene-3β,7β,16α,17β-tetrol is Form VIβ 3β-tetrol.

64. The crystalline androst-5-ene-3β,7β,16α,17β-tetrol of embodiment 1 or 2, the composition of embodiment 26 or the method of any one of embodiments 40, 42 or 48-58 wherein the solid state form or crystalline form of androst-5-ene-3β,7β,16α,17β-tetrol is Form VIIβ 3β-tetrol.

65. The crystalline androst-5-ene-3β,7β,16α,17β-tetrol of embodiment 1 or 2, the composition of embodiment 26 or the method of any one of embodiments 40, 42 or 48-58 wherein the solid state form or crystalline form of androst-5-ene-3β,7β,16α,17β-tetrol is Form VIIIβ 3β-tetrol.

66. The crystalline androst-5-ene-3β,7β,16α,17β-tetrol of embodiment 1 or 2, the composition of embodiment 26 or the method of any one of embodiments 40, 42 or 48-58 wherein the solid state form or crystalline form of androst-5-ene-3β,7β,16α,17β-tetrol is Form IXβ 3β-tetrol.

67. The crystalline androst-5-ene-3β,7β,16α,17β-tetrol of embodiment 1 or 2, the composition of embodiment 26 or the method of any one of embodiments 40, 42 or 48-58 wherein the solid state form or crystalline form of androst-5-ene-3β,7β,16α,17β-tetrol is Form Xβ 3β-tetrol.

1A. A product wherein the product is a solid state form of androst-5-ene-3β,7β,16α,17β-tetrol obtained by the process comprising, consisting essentially of or consisting of (1) dissolving 3β-tetrol in wet ethanol between about the boiling point of the solution at ambient pressure to ambient temperature to provide an ethanolic solution and (2) admixing the ethanolic solution with acetonitrile.

2A. The product of embodiment 1A wherein the ethanolic solution is at a temperature between about 40° C. to about the boiling point of the ethanolic solution at ambient pressure immediately prior to said admixing with acetonitrile.

3A. The product of embodiment 2A wherein the acetonitrile of said admixing has a volume about equal to that of the ethanolic solution.

4A. The product of embodiment 3A wherein wet ethanol has a water content of between about 5% to about 10% by volume.

5A. The product of embodiment 3A wherein the concentration of 3β-tetrol in the ethanolic solution immediately prior to said admixing is between about 50 mg/mL to about 100 mg/mL.

6A. The product of embodiment 4A wherein 3β-tetrol of said dissolving was prepared according to Example 7.

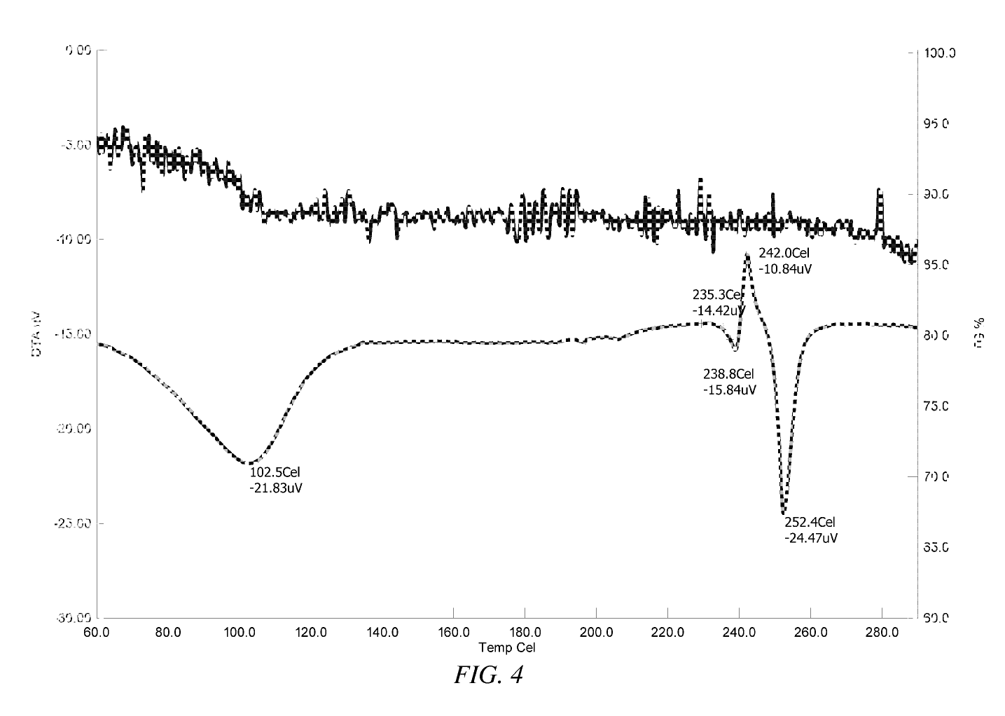

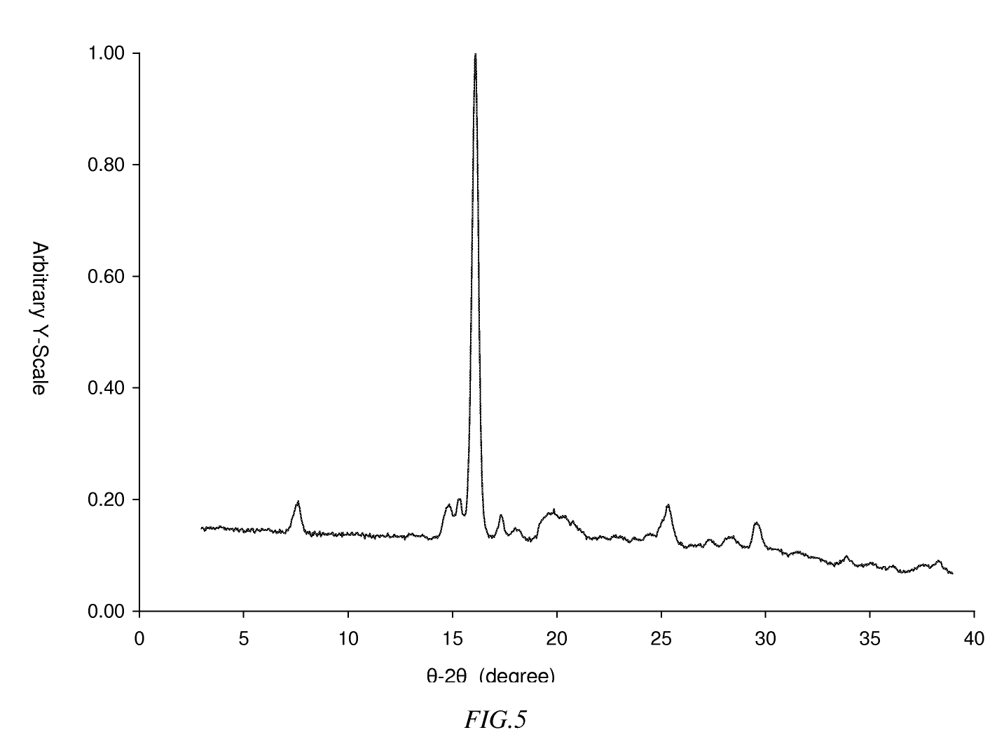

7A. The product of embodiment 1A wherein the solid state form is characterized by DTA-TGA thermograms substantially identical to FIG. 4.

8A. A product wherein the product is a solid state form of androst-5-ene-3β,7β,16α,17β-tetrol obtained by the process comprising, consisting essentially of or consisting of (1) adding seed crystal of Form VIIβ 3β-tetrol to a solution of androst-5-ene-3β,7β,16α,17β-tetrol in an wet ethanol-acetonitrile solvent mixture at a temperature at which the seed crystal does not dissolve upon its addition to the androst-5-ene-3β,7β,16α,17β-tetrol solution and (2) reducing the temperature of the mixture after said addition.

9A. The product of embodiment 8A wherein the wet ethanol of said solvent mixture has a water content of between about 5% to about 10% by volume.

10A. The product of embodiment 8A wherein the solvent mixture is about equal volumes of 90% ethanol and acetonitrile at between about 40° C. to about 70° C.

11A. The product of embodiment 8A wherein the concentration of androst-5-ene-3β,7β,16α,17β-tetrol in the wet ethanol-acetonitrile solvent mixture is between about 50 mg/mL to about 100 mg/mL.

12A. The product of embodiment 8A wherein the seed crystal of said addition is prepared according to Example 9.

13A. The product of embodiment 25A wherein androst-5-ene-3β,7β,16α,17β-tetrol of said solution is prepared according to Example 10.

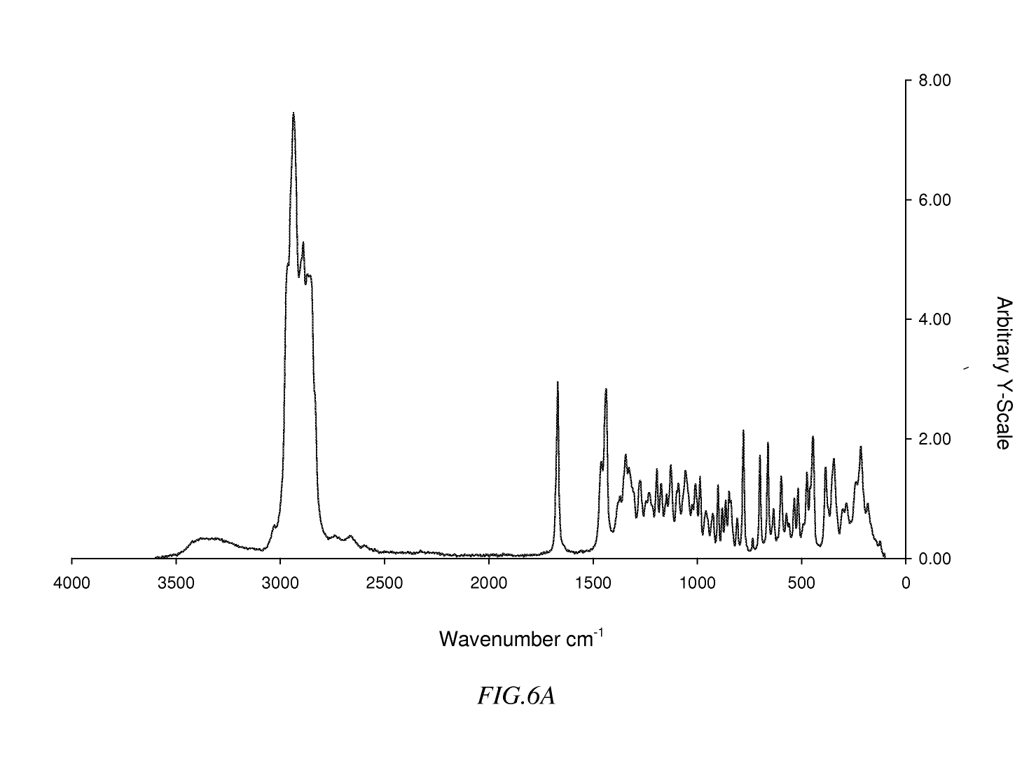

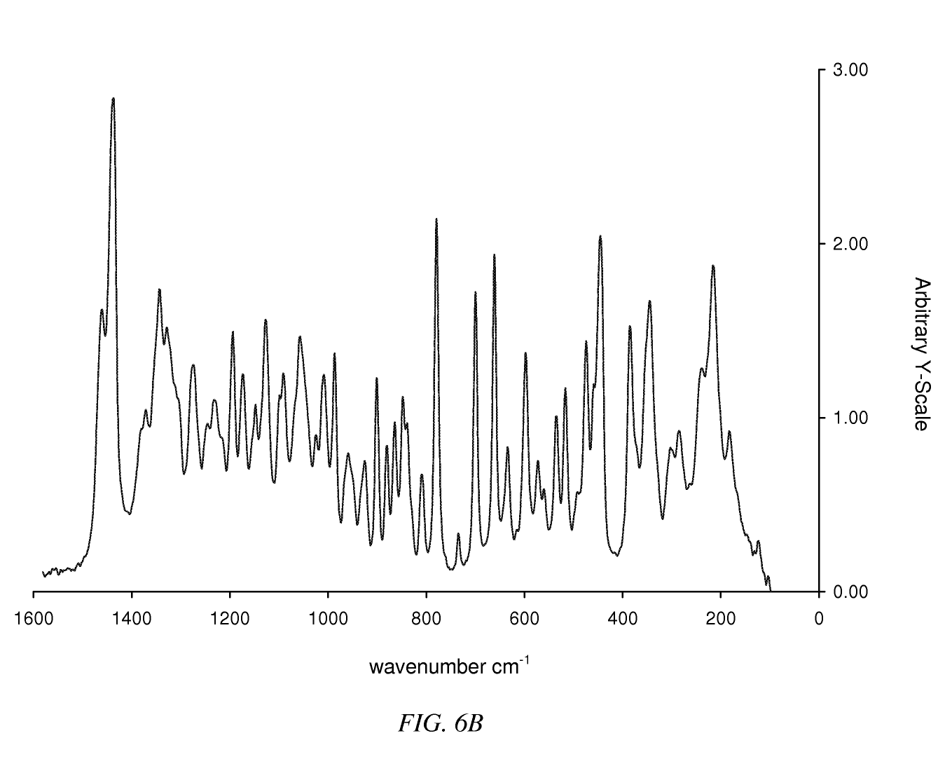

14A. The product of embodiment 8A characterized by a solid infrared Raman spectrum substantially identical to FIG. 6A or 6B.

15A. The product of embodiment 8A characterized by an X-ray powder diffraction pattern substantially identical to FIG. 5.

16A. The product of embodiment 8A characterized by an X-ray powder diffraction pattern substantially identical to FIG. 5 and a solid infrared Raman spectrum substantially identical to FIG. 6B.

17A. The product of embodiment 8A wherein the XRPD pattern has three or more prominent peaks of Table 4, optionally having at least 10% relative intensity.

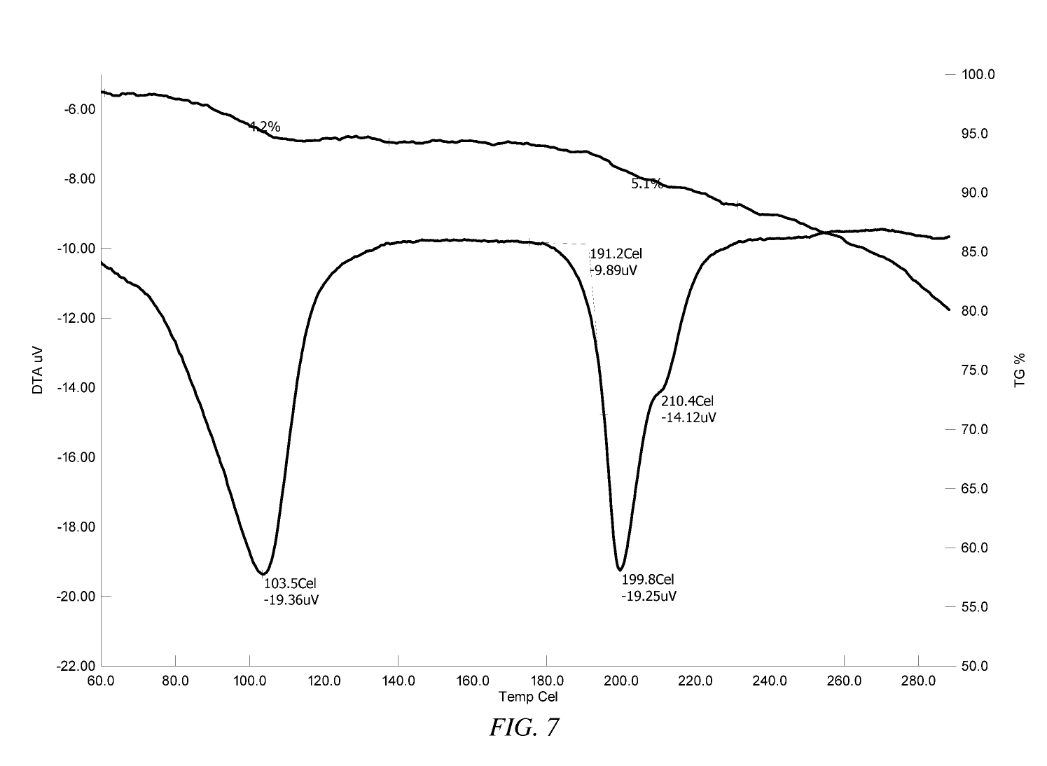

18A. The product of embodiment 8A wherein the solid state form is characterized by DTA-TG thermograms substantially identical to FIG. 7 and optionally by an X-ray powder diffraction pattern substantially identical to FIG. 5 or a solid infrared Raman spectrum substantially identical to FIG. 6B.

19A. A product wherein the product is a solid state form of androst-5-ene-3β,7β,16α,17β-tetrol obtained by the process comprising, consisting essentially of or consisting of (1) heating a mixture of 3β-tetrol, ethanol and heptane at a temperature sufficient to obtain a homogeneous solution between about the boiling point of the mixture at ambient pressure to ambient temperature and (2) reducing the temperature of the solution after said heating.

20A. The product of embodiment 19A wherein the ethanol of said heating is denatured ethanol having a water content of 5% by volume or less.

21A. The product of embodiment 19A wherein the mixture of said heating is prepared by admixing ethanol and heptane in about equal volume with 3β-tetrol and wherein said heating is to a temperature of between about 40° C. to about 70° C.

22A. The product of embodiment 19A wherein the concentration of 3β-tetrol in the ethanol-heptane solvent mixture is between about 50 mg/mL to about 100 mg/mL.

23A. The product of embodiment 22A wherein 3β-tetrol of said solution is prepared according to Example 10.

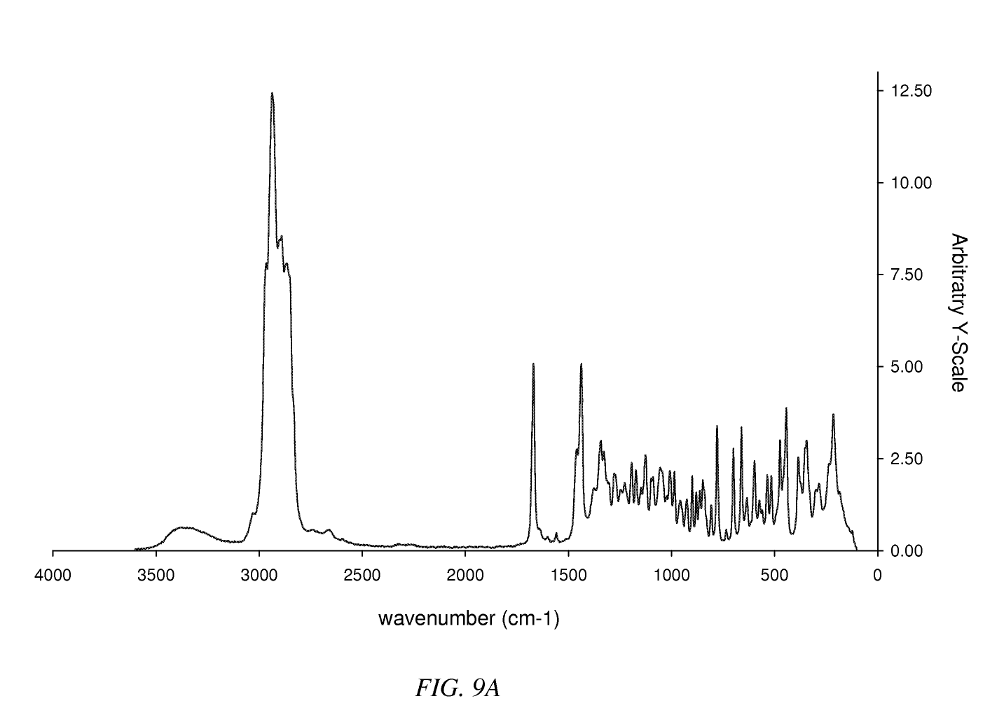

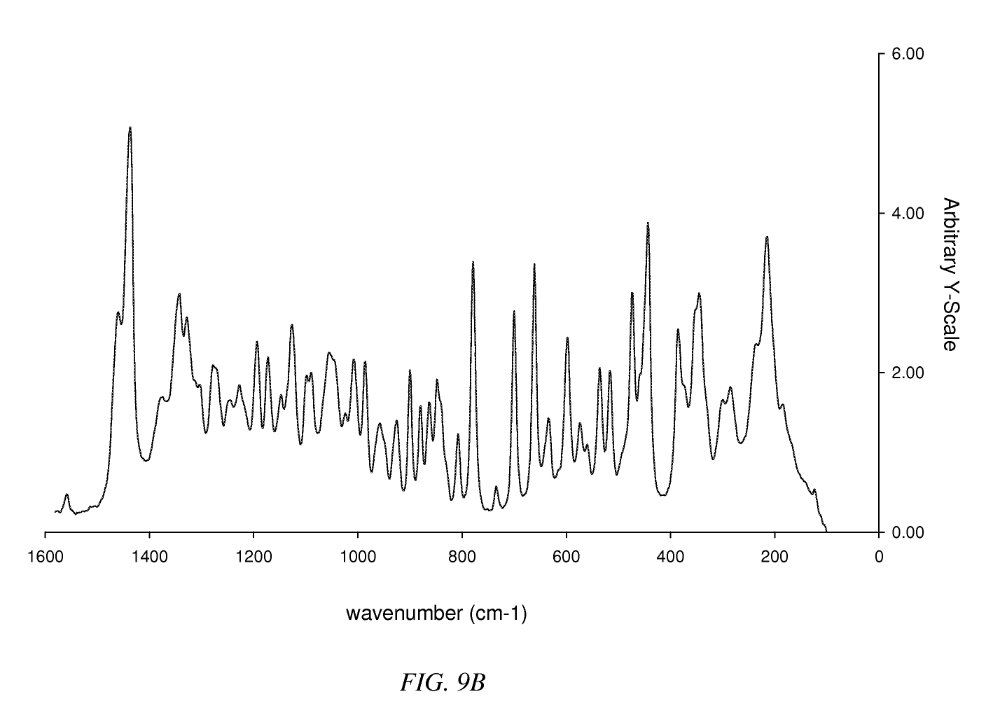

24A. The product of embodiment 19A characterized by a solid infrared Raman spectrum substantially identical to FIG. 9A or 9B.

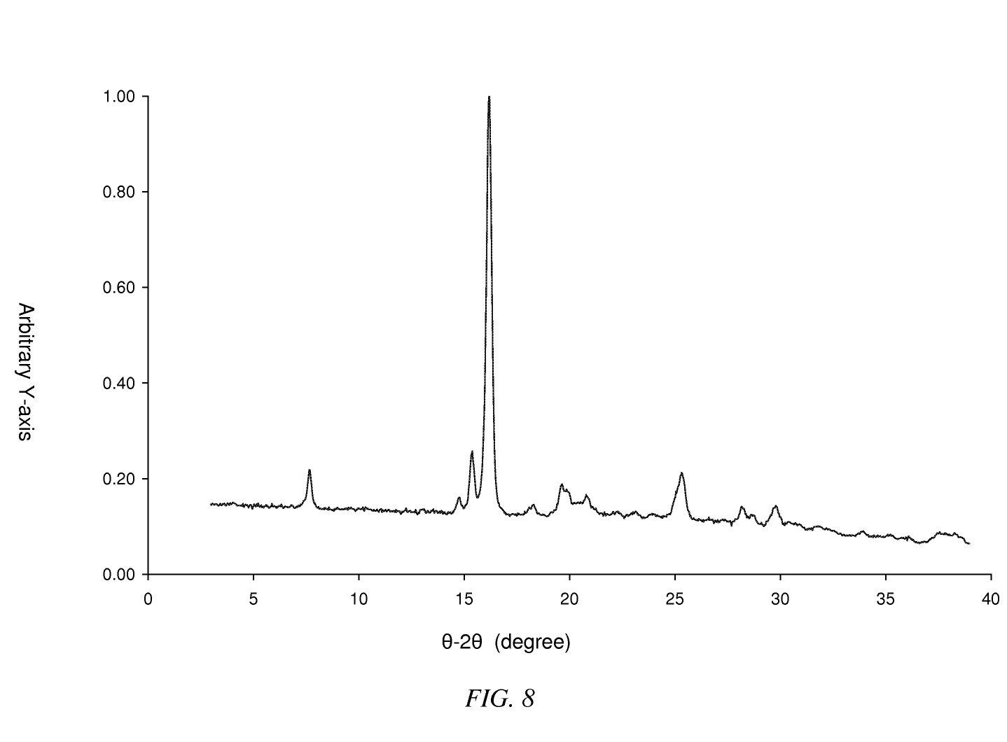

25A. The product of embodiment 19A characterized by an X-ray powder diffraction pattern substantially identical to FIG. 8.

26A. The product of embodiment 19A characterized by an X-ray powder diffraction pattern substantially identical to FIG. 8 and a solid infrared Raman spectrum substantially identical to FIG. 9B.

27A. The product of embodiment 19A wherein the XRPD pattern has three or more prominent peaks of Table 6, optionally having at least 10% relative intensity.

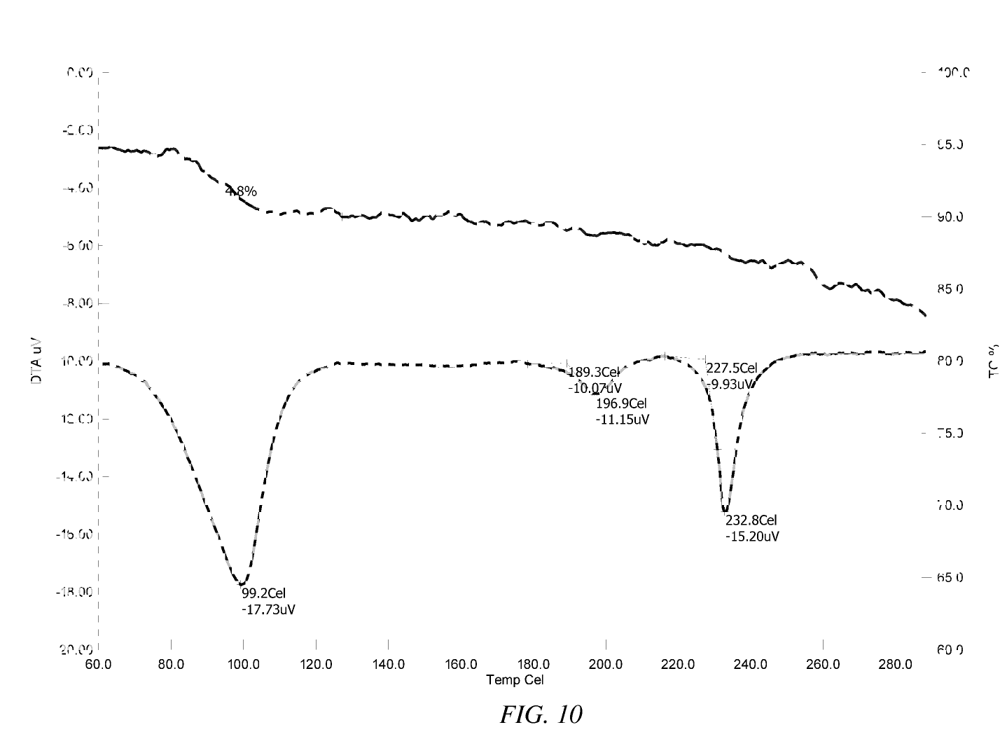

28A. The product of embodiment 19A wherein the solid state form is characterized by DTA-TGA thermograms substantially identical to FIG. 10 and optionally by an X-ray powder diffraction pattern substantially identical to FIG. 8 or a solid infrared Raman spectrum substantially identical to FIG. 9B.

28A. A product wherein the product is a solid state form of androst-5-ene-3β,7β,16α,17β-tetrol obtained by the process comprising, consisting essentially of or consisting of (1) heating a mixture of 3β-tetrol and methanol at a temperature sufficient to obtain a homogeneous solution between about the boiling point of the mixture at ambient pressure to ambient temperature, (2) optionally filtering the heated solution from step (1) to remove insoluble impurities if present; and (3) reducing the temperature of the mixture after said heating or said optional filtering.

29A. The product of embodiment 28A wherein the concentration of 3β-tetrol in the heated methanol solution is between about 50 mg/mL to about 100 mg/mL.

30A. The product of embodiment 29A wherein 3β-tetrol of said methanol solution is prepared according to Example 10.

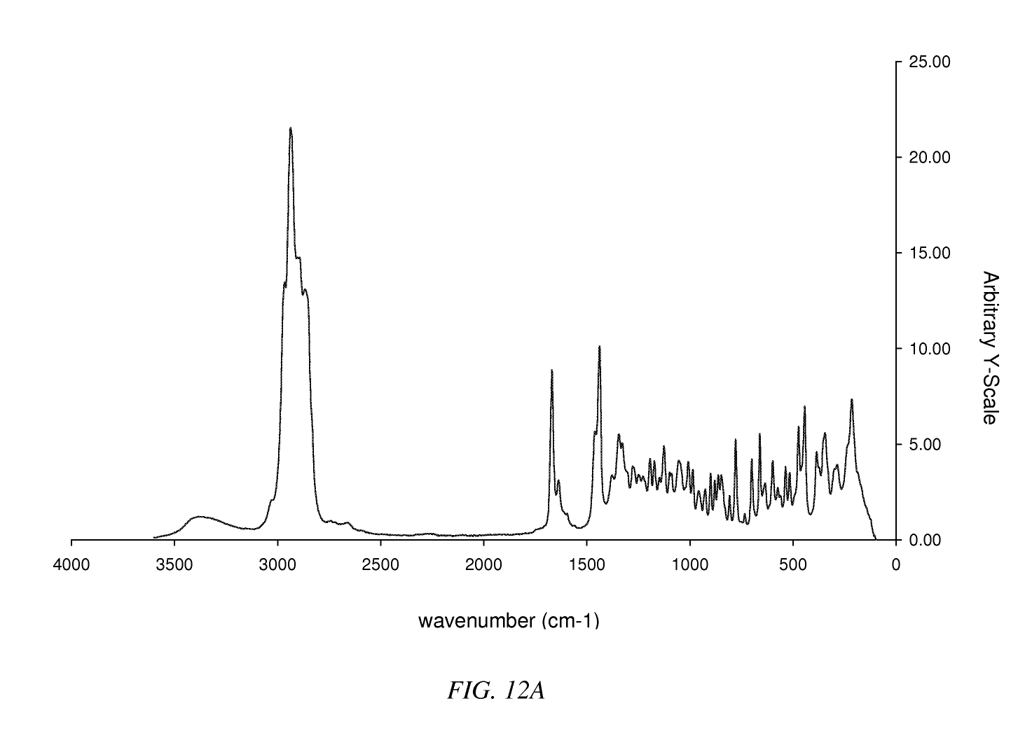

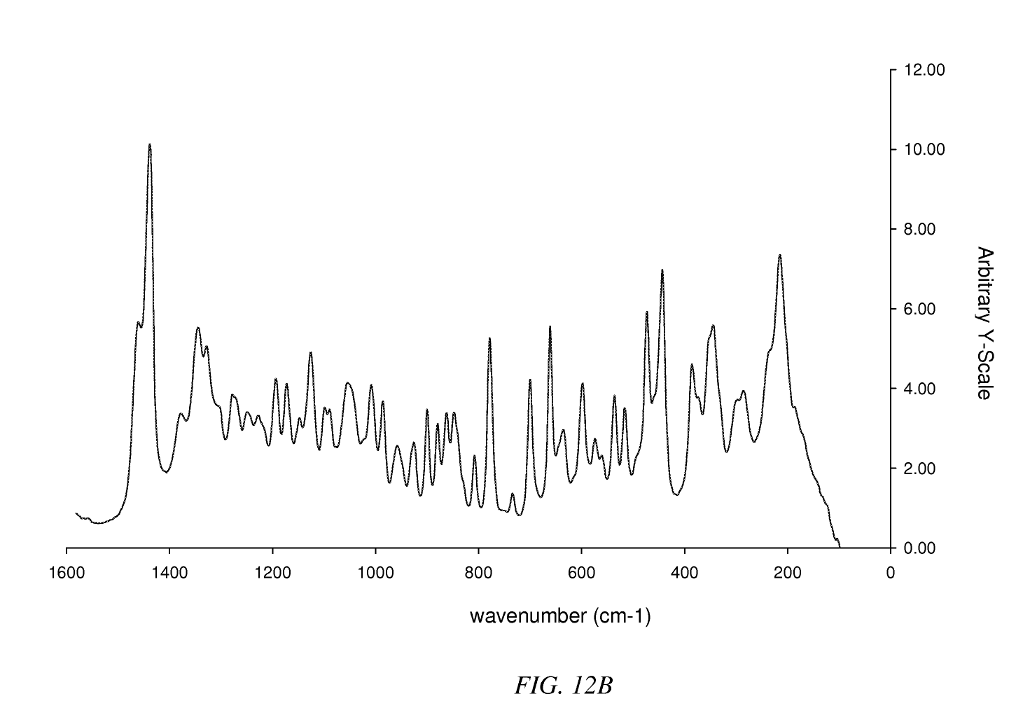

31A. The product of embodiment 28A characterized by a solid infrared Raman spectrum substantially identical to FIG. 12A or 12B.

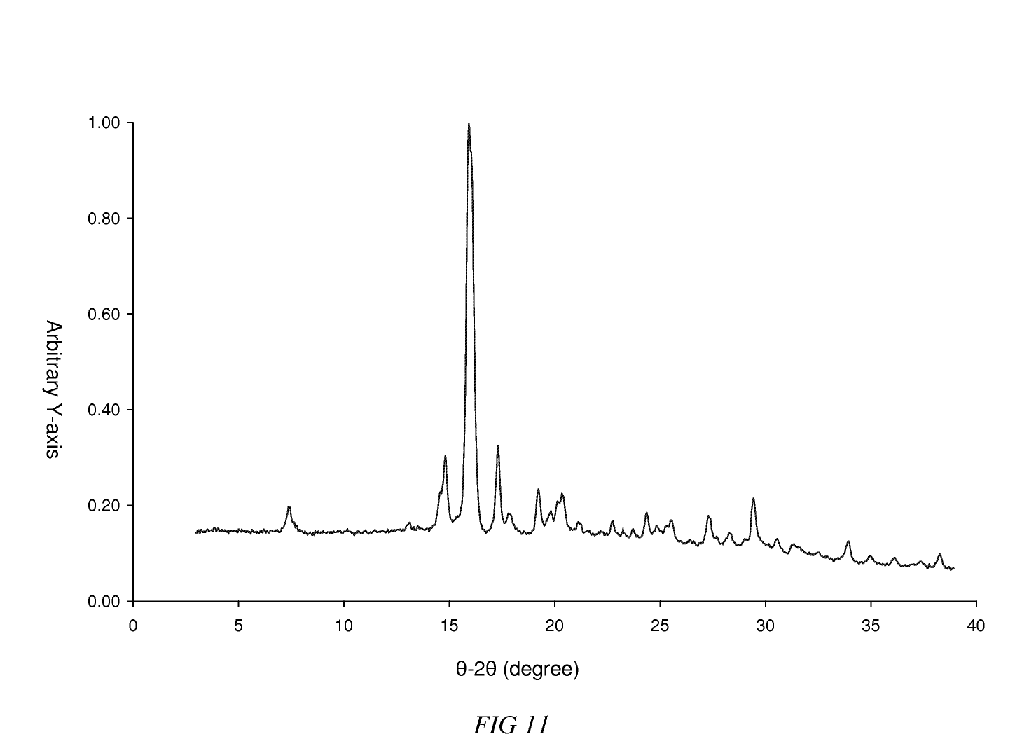

32A. The product of embodiment 28A characterized by an X-ray powder diffraction pattern substantially identical to FIG. 11.

33A. The product of embodiment 28A characterized by an X-ray powder diffraction pattern substantially identical to FIG. 11 and a solid infrared Raman spectrum substantially identical to FIG. 12B.

34A. The product of embodiment 28A wherein the XRPD pattern has by three or more prominent peaks of Table 8, optionally having at least 10% relative intensity.

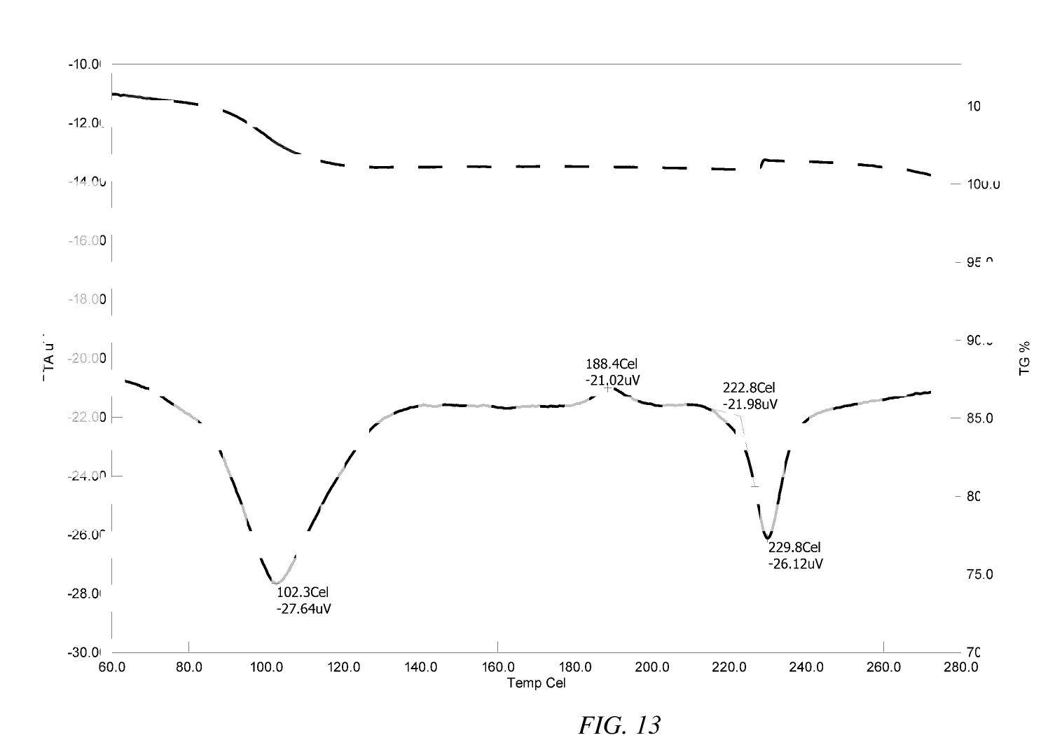

35A. The product of embodiment 28A wherein the solid state form is characterized by DTA-TGA thermograms substantially identical to FIG. 13 and optionally by an X-ray powder diffraction pattern substantially identical to FIG. 11 or a solid infrared Raman spectrum substantially identical to FIG. 12B.

36A. A product wherein the product is a solid state form of androst-5-ene-3β,7β,16α,17β-tetrol obtained by the process comprising, consisting essentially of or consisting of (1) dissolving 3β-tetrol in methanol to provide a substantially homogeneous methanolic solution at room temperature, (2) filtering the methanolic solution to remove non-dissolved material; and (3) reducing the volume of the filtered methanolic solution.

37A. The product of embodiment 36A wherein 3β-tetrol of said dissolving is prepared according to Example 11.

38A. The product of embodiment 37A wherein said filtering is through a filter having porosity of between about 0.45 micron to about 0.2 micron.

39A. The product of embodiment 93A wherein said volume reduction is by distillation of solvent under reduced pressure at or below room temperature to a volume at which precipitation is initiated.

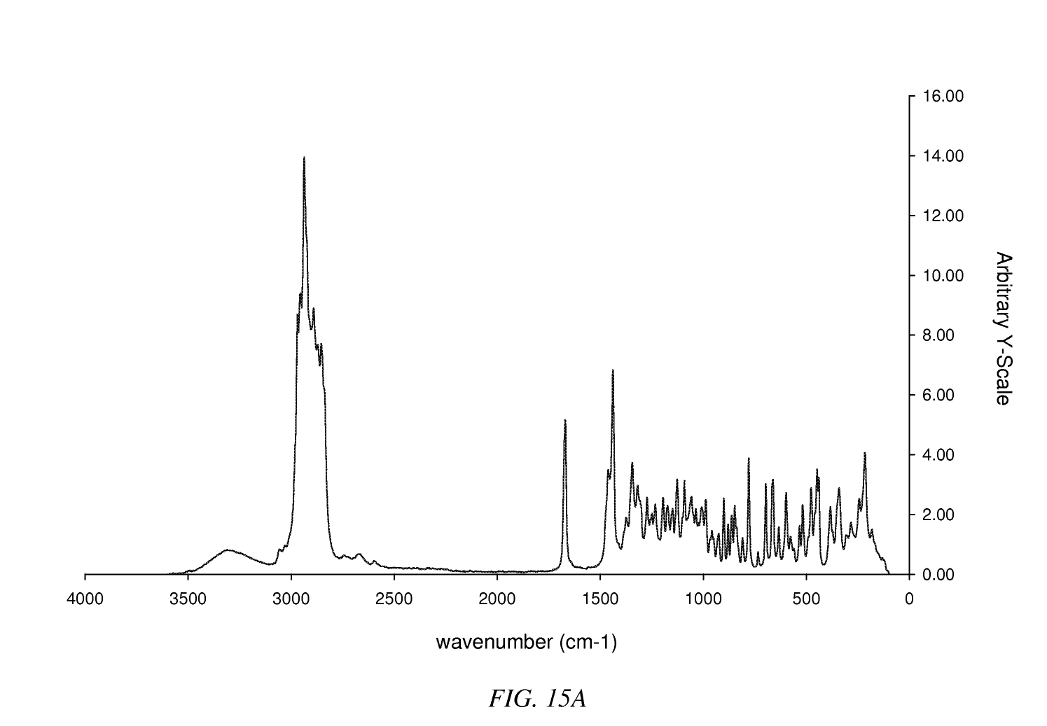

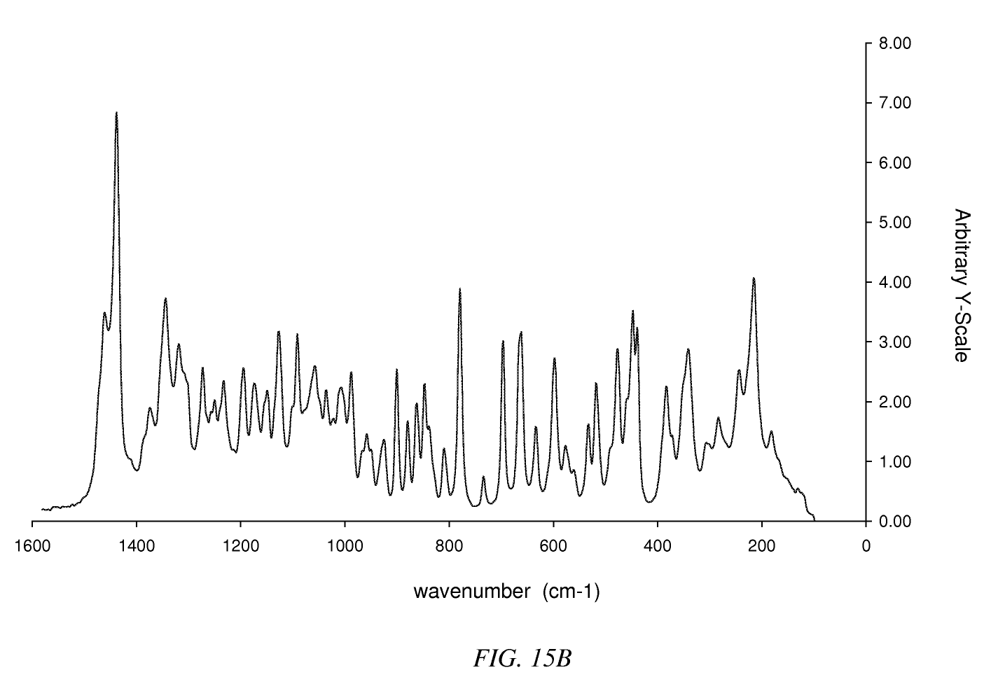

40A. The product of embodiment 36A characterized by a solid infrared Raman spectrum substantially identical to FIG. 15A or 15B.

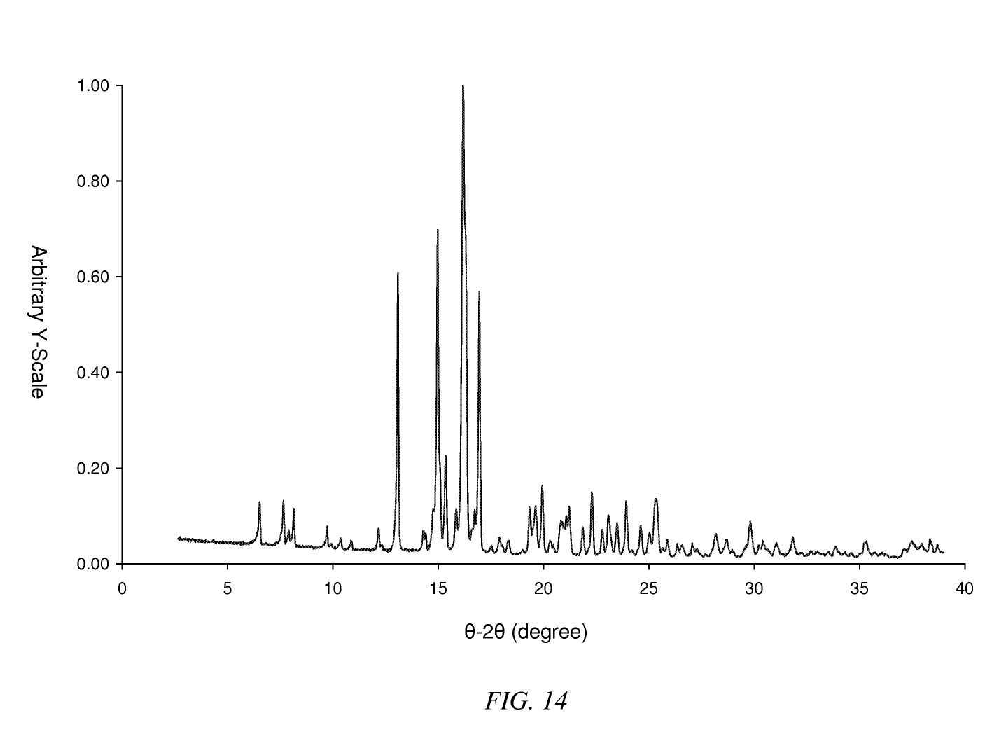

41A. The product of embodiment 36A characterized by an X-ray powder diffraction pattern substantially identical to FIG. 14.

42A. The product of embodiment 36A characterized by an X-ray powder diffraction pattern substantially identical to FIG. 14 and a solid infrared Raman spectrum substantially identical to FIG. 15B.

43A. The product of embodiment 36A wherein the XRPD pattern has three or more prominent peaks of Table 10, optionally having at least 10% relative intensity.

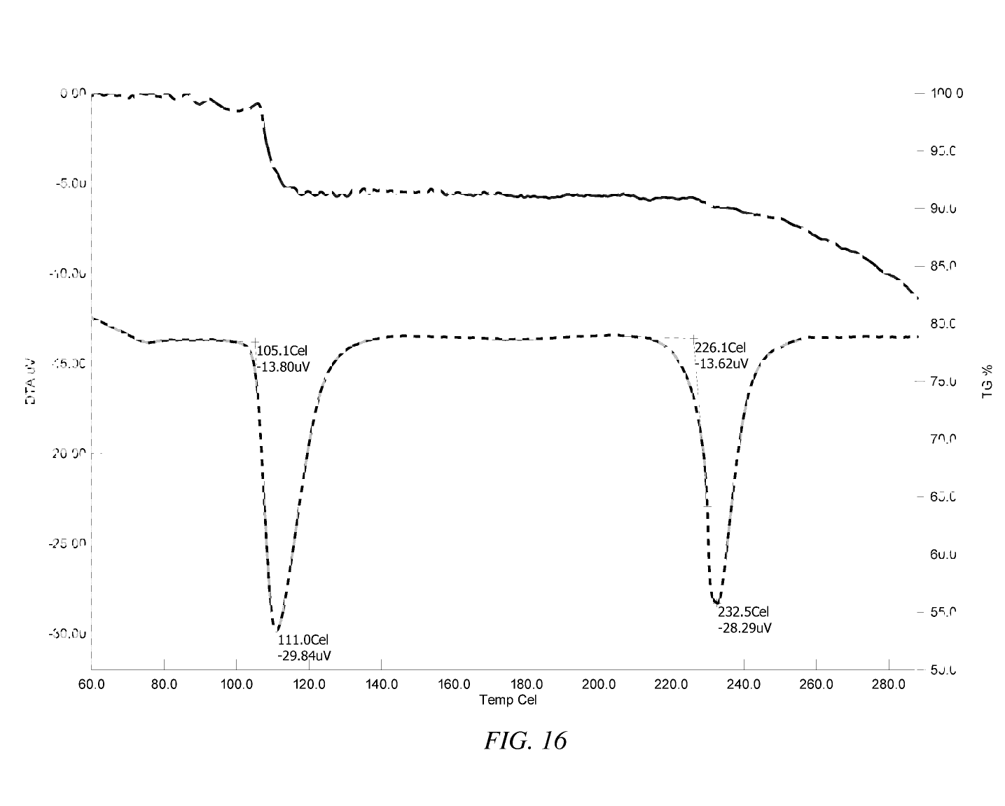

44A. The product of embodiment 36A wherein the solid state form is characterized by DTA-TGA thermograms substantially identical to FIG. 16 and optionally by an X-ray powder diffraction pattern substantially identical to FIG. 14 or a solid infrared Raman spectrum substantially identical to FIG. 15B.

1B. A product wherein the product is a solid state form of androst-5-ene-3β,7β,16α,17β-tetrol obtained by the process comprising, consisting essentially of or consisting of (1) heating 3β-tetrol in crystalline form until a melt is obtained and (2) reducing the temperature of the melt until its solidification.

2B. The product of embodiment 1B wherein the crystalline form of said heating is Form IIβ 3β-tetrol.

3B. The product of embodiment 2B wherein the crystalline form is heated to between about 260° C. to 290° C. using a temperature ramp of about 10° C./min.

4B. The product of embodiment 3B characterized by a DTA thermogram obtained using a temperature ramp of about 10° C./min having a single prominent endotherm transition within the temperature range of 230° C.-270° C. wherein the endotherm transition is centered between about 250-254° C. and is associated with negligible % weight loss in TGA thermogram.

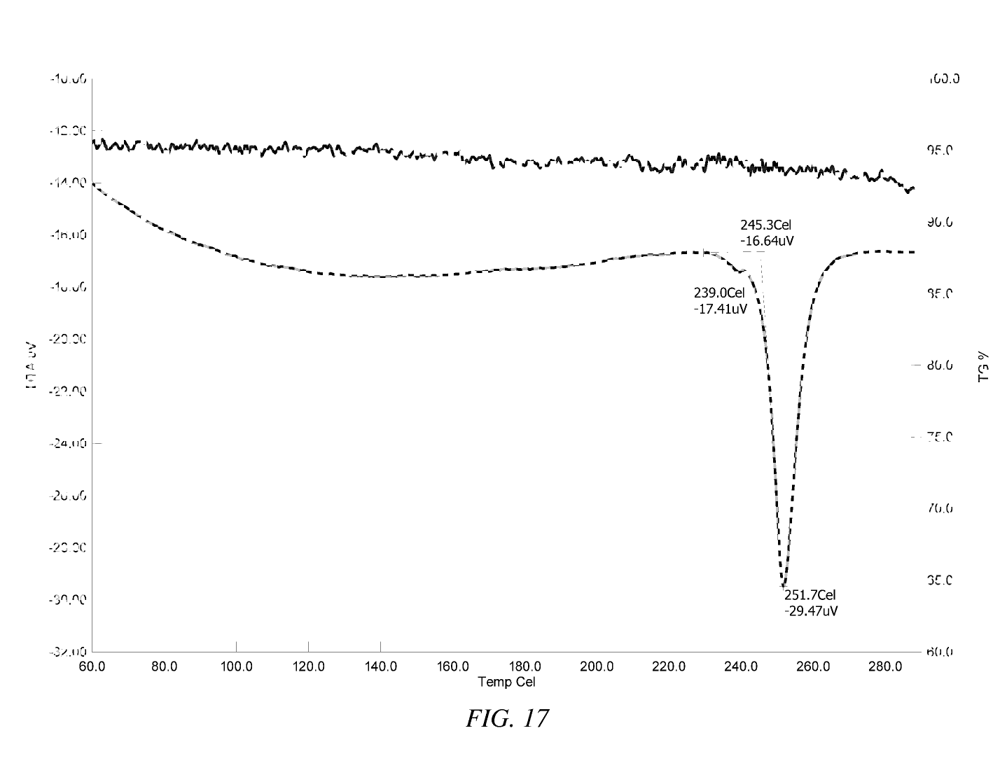

5B. The product of embodiment 1B characterized by DTA-TGA thermograms of FIG. 17.

6B. A product wherein the product is a solid state form of androst-5-ene-3β,7β,16α,17β-tetrol obtained by the process comprising, consisting essentially of or consisting of (1) dissolving 3β-tetrol in anhydrous methanol to provide a substantially homogeneous methanolic solution at room temperature, (2) filtering the methanolic solution to remove non-dissolved material, (3) reducing the volume of the filtered methanolic solution to provide a slurry; and (4) admixing the slurry with EtOAc.

7B. The product of embodiment 6B wherein the 3β-tetrol of said dissolving is prepared according to Example 2.

8B. The product of embodiment 7B wherein said filtering is through a filter having porosity from about 11 micron to about 0.2 micron.

9B. The product of embodiment 6B wherein about 2:1 to about 4:1 v/w EtOAc is admixed with the 3β-tetrol slurry.

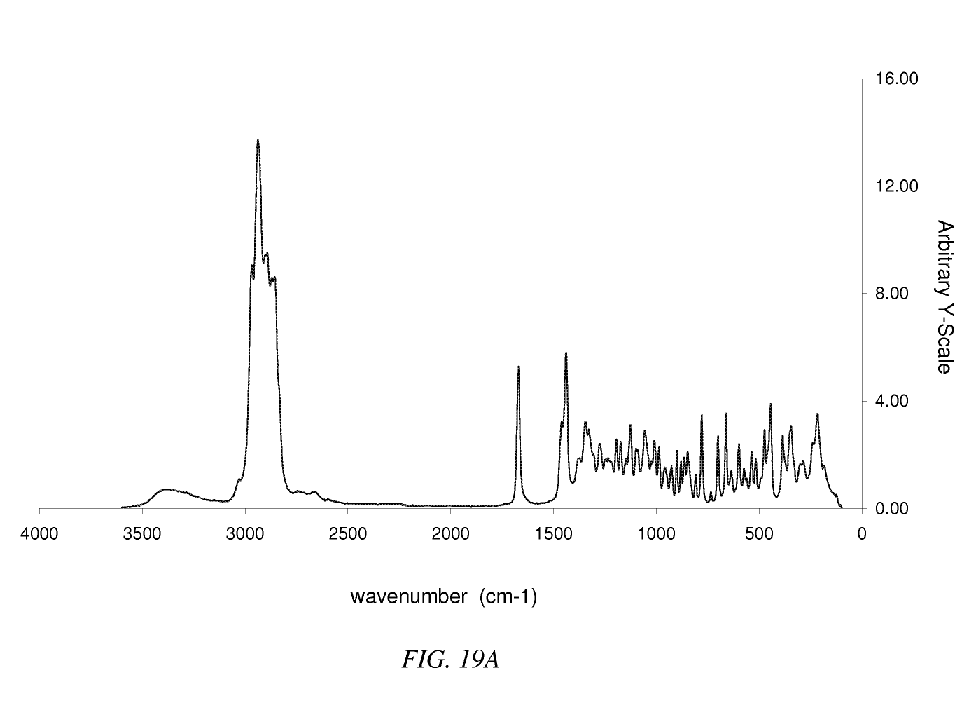

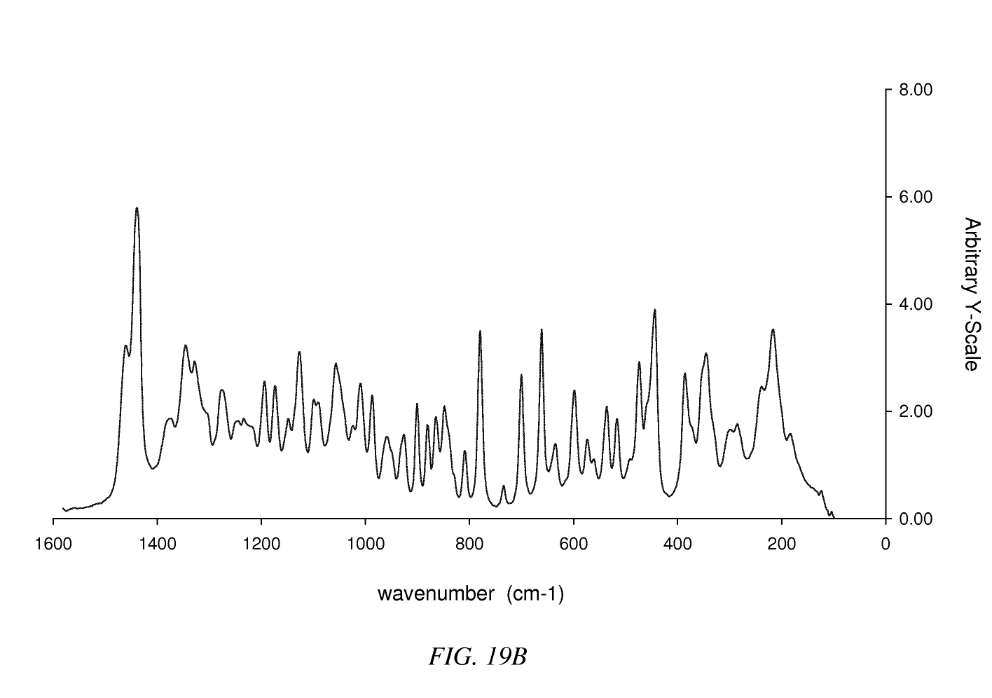

10B. The product of embodiment 6B characterized by a solid infrared Raman spectrum substantially identical to FIG. 19A or 19B.

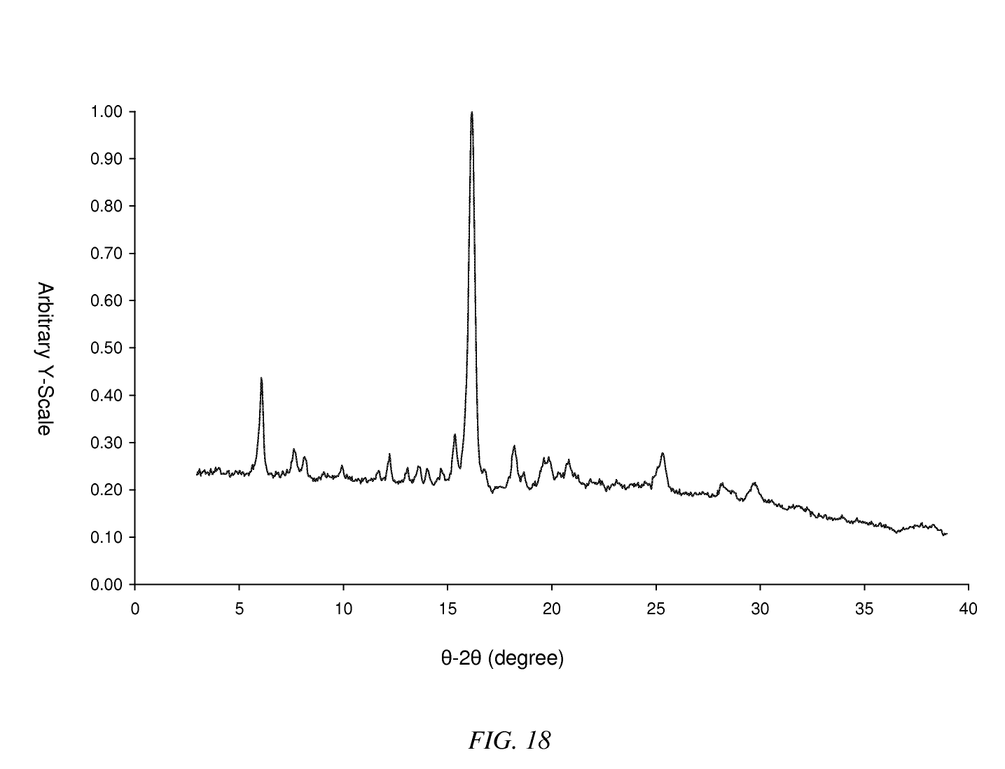

11B. The product of embodiment 6B characterized by an X-ray powder diffraction pattern substantially identical to FIG. 18.

12B. The product of embodiment 6B characterized by an X-ray powder diffraction pattern substantially identical to FIG. 18 and a solid infrared Raman spectrum substantially identical to FIG. 19B.

13B. The product of embodiment 6B wherein the XRPD pattern has three or more prominent XRPD peaks of Table 12.

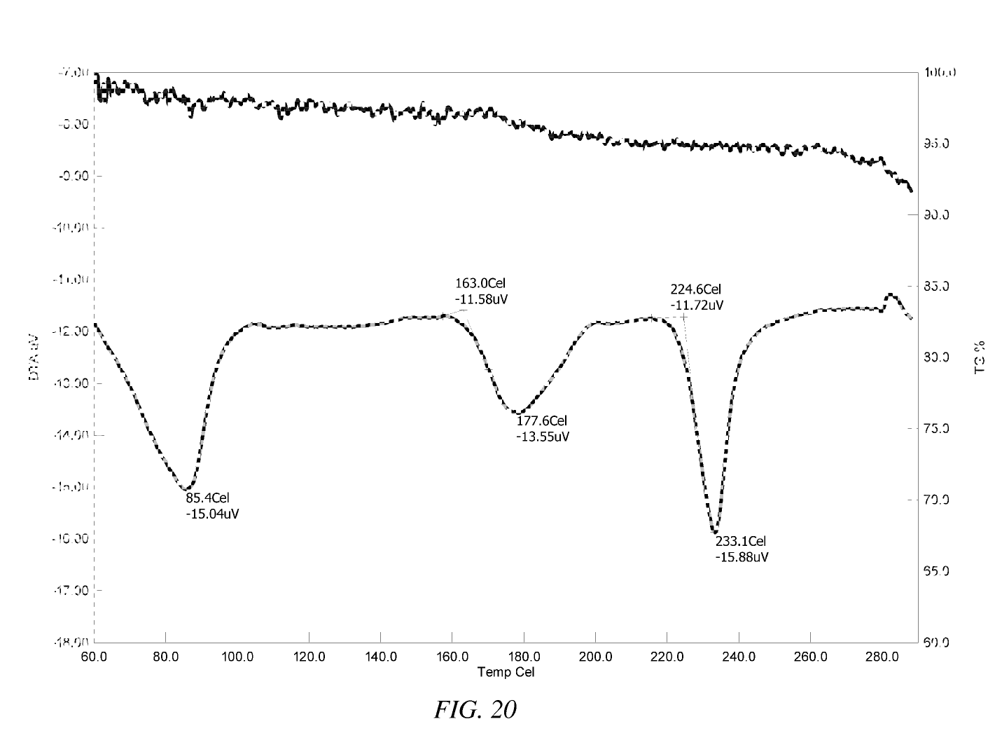

14B. The product of embodiment 6B wherein the solid state form is characterized by DTA-TGA thermograms substantially identical to FIG. 20 and optionally by an X-ray powder diffraction pattern substantially identical to FIG. 18 or a solid infrared Raman spectrum substantially identical to FIG. 19B.

15B. A product wherein the product is a solid state form of androst-5-ene-3β,7β,16α,17β-tetrol obtained by the process comprising, consisting essentially of or consisting of (1) dissolving 3β-tetrol in ethanol to provide a ethanolic solution at room temperature, (2) admixing the ethanolic solution with water to induce precipitation; and (3) heating the collected precipitate in vacuo to provide an anhydrate.

16B. The product of embodiment 15B wherein the 3β-tetrol of said dissolving is prepared according to Example 10.

17B. The product of embodiment 15B wherein ethanol of said dissolving is denatured ethanol having a water content of 5% by volume or less.

18B. The product of embodiment 15B wherein the concentration of 3β-tetrol in the ethanolic solution is between about 100 mg/mL to about 200 mg/mL.

19B. The product of embodiment 18B wherein about an equal volume of water is admixed at room temperature with the ethanolic solution.

20B. The product of embodiment 15B wherein said precipitate heating is at about 100° C. or more, provided said heating does not melt the collected precipitate, under 0.2 torr or less vacuum and for a duration that provides the anhydrate without detectable decomposition by visual inspection.

21B. The product of embodiment 20B wherein said heating is at about 100° C. under 0.2 torr vacuum when about 5 g of precipitate is collected.

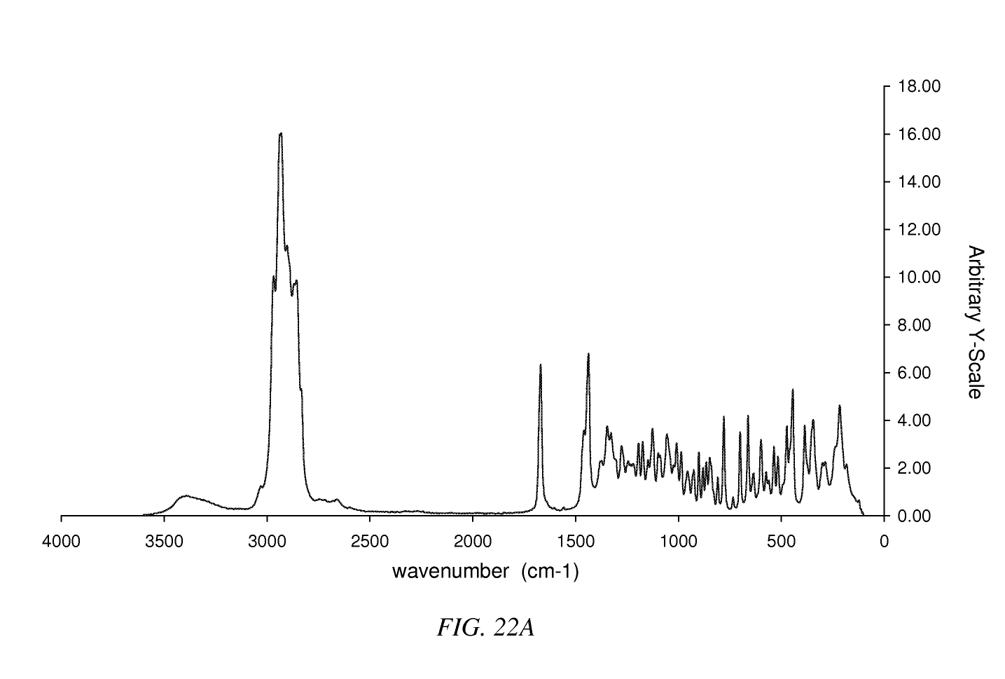

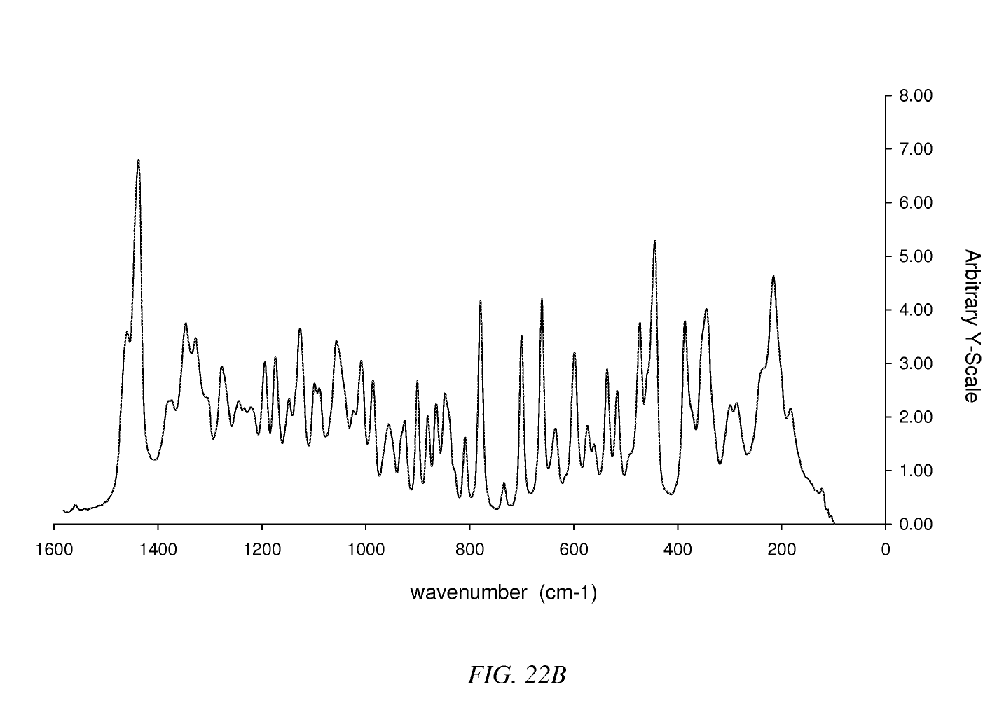

22B. The product of embodiment 15B characterized by a solid infrared Raman spectrum substantially identical to FIG. 22A or 22B.

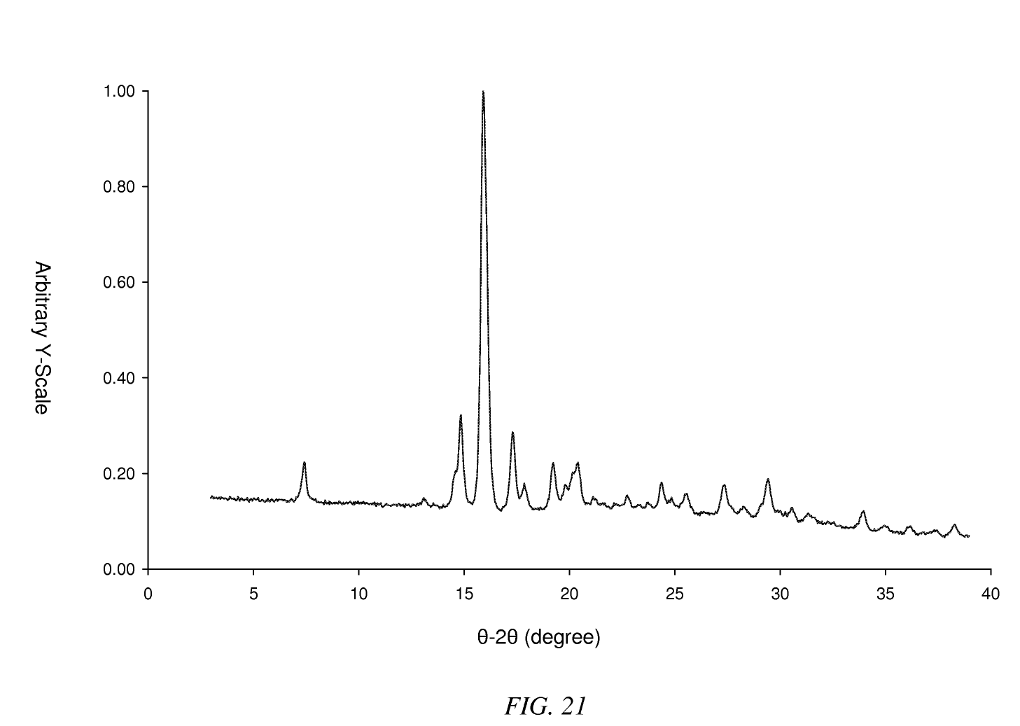

23B. The product of embodiment 15B characterized by an X-ray powder diffraction pattern substantially identical to FIG. 21.

24B. The product of embodiment 15B characterized by an X-ray powder diffraction pattern substantially identical to FIG. 21 and a solid infrared Raman spectrum substantially identical to FIG. 22B.

25B. The product of embodiment 15B wherein the XRPD pattern has three or more prominent peaks of Table 14, optionally having at least 10% relative intensity.

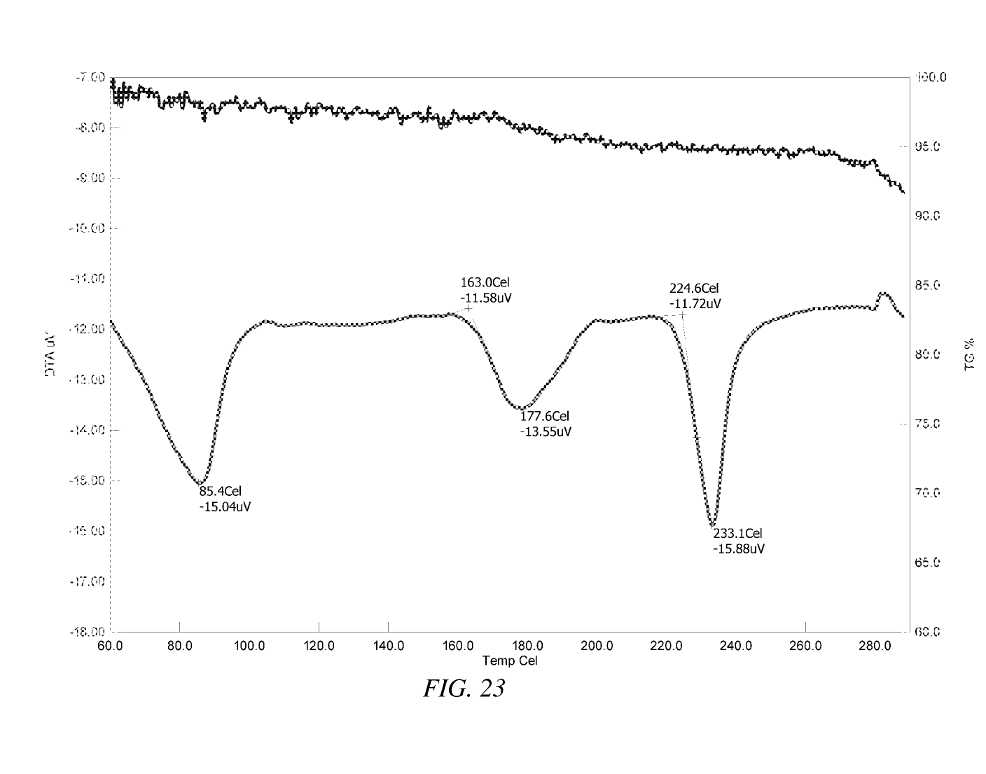

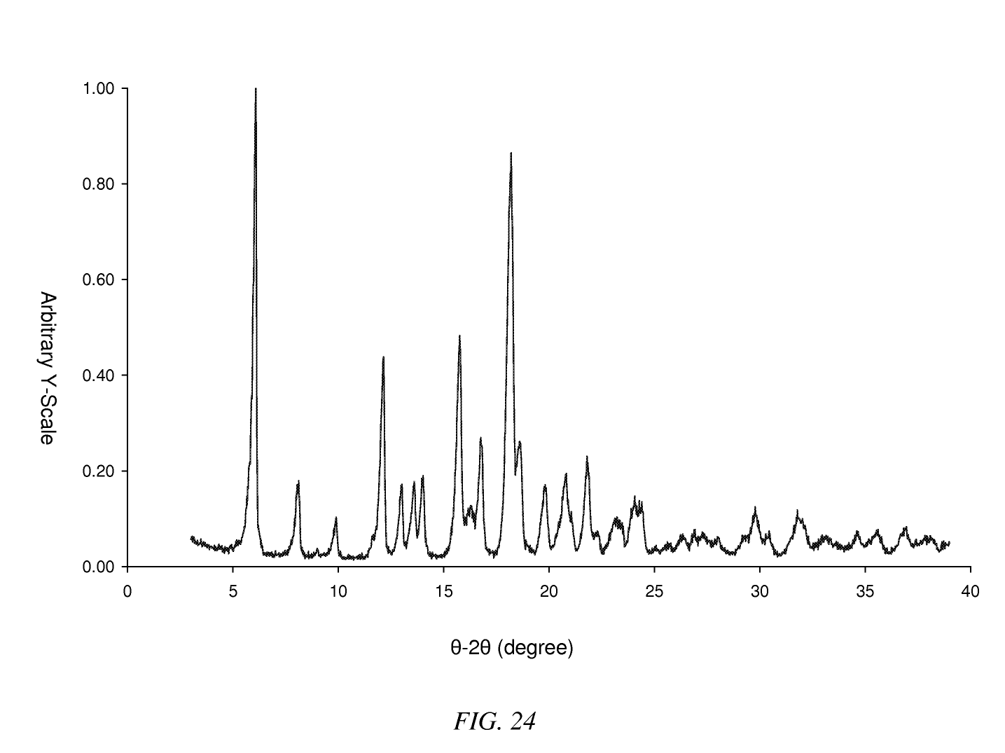

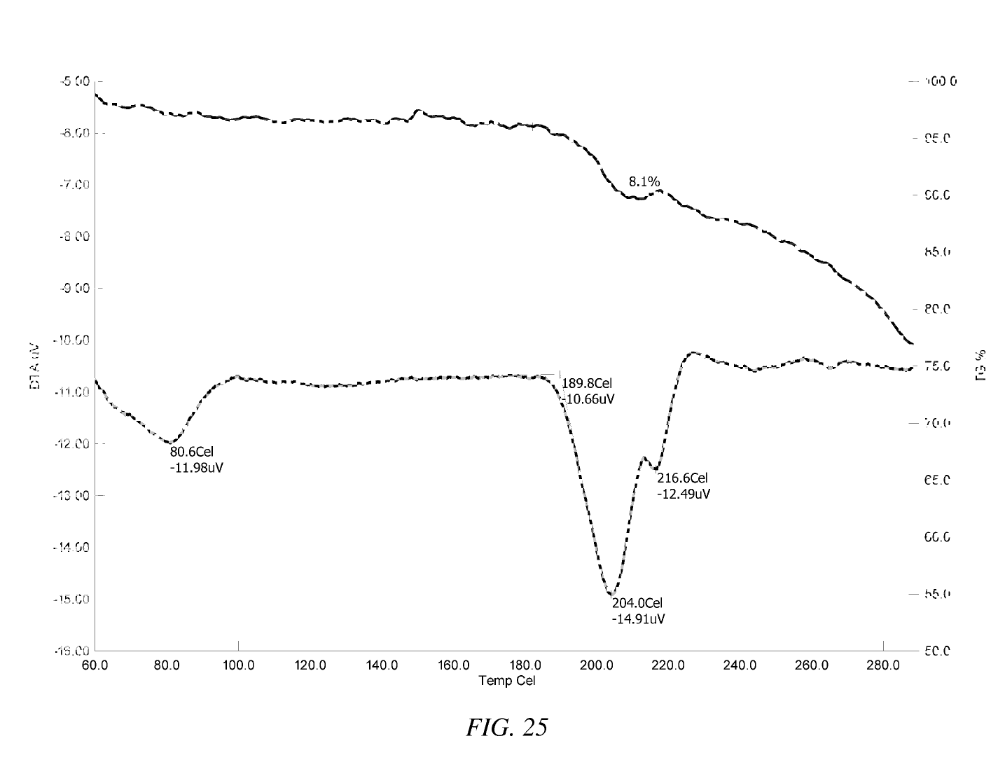

26B. The product of embodiment 15B wherein the solid state form is characterized by DTA-TGA thermograms substantially identical to FIG. 23 and optionally by an X-ray powder diffraction pattern substantially identical to FIG. 18 or a solid infrared Raman spectrum substantially identical to FIG. 19B.

27B. A product wherein the product is a solid state form of androst-5-ene-3β,7β,16α,17β-tetrol obtained by the process comprising, consisting essentially of or consisting of (1) dissolving 3β-tetrol in absolute ethanol to provide an ethanolic solution at room temperature, (2) admixing the ethanolic solution with ethyl acetate and (3) maintaining the ethanolic solution protected from atmospheric moisture at room temperature for a duration until a majority of the initial mass of 3β-tetrol of said dissolving precipitates.

28B. The product of embodiment 27B wherein the 3β-tetrol of said dissolving is prepared according to Example 10.

29B. The product of embodiment 27B wherein the concentration of 3β-tetrol in the ethanolic solution is between about 100 mg/mL to about 200 mg/mL.

30B. The product of embodiment 29B wherein about twice the volume of EtOAc is admixed with the ethanolic solution.

31B. The product of embodiment 29B wherein the concentration of 3β-tetrol in the ethanolic solution is about 100 mg/mL and said duration of said maintaining is at least 60 hr.

32B. The product of embodiment 27B characterized by an X-ray powder diffraction pattern substantially identical to FIG. 21

33B. The product of embodiment 27B wherein the XRPD pattern has three or more prominent peaks of Table 16, optionally having at least 10% relative intensity.

34B. The product of embodiment 27B wherein the solid state form is characterized by DTA-TG thermograms substantially identical to FIG. 23 and optionally by an X-ray powder diffraction pattern substantially identical to FIG. 21 or a solid infrared Raman spectrum substantially identical to FIG. 19B.

1C. A composition comprising, consisting essentially of or consisting of one or more, typically one or two, crystalline forms of androst-5-ene-3β,7β,16α,17β-tetrol, obtained by process(es) selected from the group consisting of embodiment 1A, 8A, 19A, 28A and 36A, and one or more excipients.

2C. A composition comprising, consisting essentially of or consisting of one or more, typically one or two, crystalline forms of androst-5-ene-3β,7β,16α,17β-tetrol, obtained by process(es) selected from the group consisting of embodiment 1B, 5B, 14B and 26B, and one or more excipients.

3C. A method of preparing a liquid formulation comprising, consisting essentially of or consisting of admixing one or more, typically one or two, crystalline forms of androst-5-ene-3β,7β,16α,17β-tetrol, obtained by process(es) selected from the group consisting of embodiment 1A, 8A, 19A, 28A, 36A, 1B, 5B, 14B and 26B.

4C. A method of treating an inflammation condition or disease or another condition or disease described herein, comprising administering an effective amount of a solid formulation to a subject in need thereof wherein the formulation comprises one or more, typically one or two, crystalline forms of androst-5-ene-3β,7β,16α,17β-tetrol obtained by process(es) selected from the group consisting of embodiment 1A, 8A, 19A, 28A and 36A or from the group consisting of 1B, 5B, 14B and 26B.

5C. The method of embodiment 4C wherein the inflammation condition or disease is associated with chronic, non-production inflammation.

6C. The method of embodiment 4C wherein the condition or disease is an autoimmune condition or disease.

7C. The method of embodiment 4C wherein the condition or disease is a metabolic condition or disease.

8C. The method of embodiment 6C wherein the autoimmune disease is Type 1 diabetes.

9C. The method of embodiment 6C wherein the autoimmune disease is a lupus condition such as systemic lupus erythematosus or discoid lupus or an arthritis condition such as rheumatoid arthritis.

10C. The method of embodiment 4C wherein the condition or disease is an inflammatory bowel disease such as ulcerative colitis or Crohn's disease (regional enteritis).

11C. The method of embodiment 4C wherein the condition or disease is a lung inflammation condition such as cystic fibrosis, chronic obstructive pulmonary disease (COPD), acute asthma, chronic asthma, emphysema, acute bronchitis, allergic bronchitis, chronic bronchitis and fibrosing alveolitis (lung fibrosis) conditions, e.g., subepithelial fibrosis in patients having chronic bronchitis, asthma and/or COPD.

12C. The method of embodiment 4C wherein the condition or disease is a neurodegenerative condition such as Parkinson's disease or Alzheimer's disease.

13C. The method of embodiment 4C wherein the condition or disease is a hyperproliferation condition.

14C. The method of embodiment 4C wherein the condition or disease is a liver cirrhosis condition, NASH or fatty liver conditions.

15C. The method of embodiment 7C wherein the metabolic condition or disease is type 2 diabetes, obesity, insulin resistance, hyperglycemia, impaired glucose utilization or tolerance, impaired or reduced insulin synthesis.

16C. A formulation comprising, consisting essentially of or consisting of or prepared from androst-5-ene-3β,7β,16α,17β-tetrol in one or more crystalline forms and one or more pharmaceutically acceptable excipients provided that Form Iβ is not present or present as a minor component or is not present as a major component.

17C. The formulation of embodiment 16C wherein androst-5-ene-3β,7β,16α,17β-tetrol is present in the formulation as a crystalline monohydrate or a crystalline dihydrate or a mixture thereof.

18C. The formulation of embodiment 17C wherein androst-5-ene-3β,7β,16α,17β-tetrol is present in the formulation as a crystalline hydrate or a crystalline anhydrate of a mixture thereof.

19C. The formulation of embodiment 18C wherein the androst-5-ene-3β,7β,16α,17β-tetrol is present in the formulation as mixture of a crystalline anhydrate and a crystalline monohydrate, optionally wherein the crystalline monohydrate is a minor component selected from the group consisting of Form Iβ, Form IIβ, Form IIIβ, Form IVβ, Form Vβ or Form VIβ 3β-tetrol.

20C. The formulation of embodiment 18C wherein herein the androst-5-ene-3β,7β,16α,17β-tetrol is present in the formulation as mixture of a crystalline monohydrate and a crystalline dihydrate, optionally wherein the crystalline monohydrate is a minor component selected from the group consisting of Form Iβ, Form IIβ, Form IIIβ, Form IVβ, Form Vβ or Form VIβ 3β-tetrol.

21C. A crystalline form of 3β-tetrol wherein 3β-tetrol is (a) a powder or granule that is at least 80% pure, at least 95% pure or at least 98% pure or (b) a solution or suspension that is at least 80% pure, at least 95% pure or at least 98% pure.

22C. The crystalline form of embodiment 21C wherein 3β-tetrol is about 80%, about 85%, about 90%, about 95%, about 97% or about 98% to about 99.5% or about 99.9% pure, optionally wherein 3β-tetrol is in the form of a powder or granules, optionally wherein the powder has an average particle size of about 50 nm or about 100 nm to about 5 μm, about 10 μm or about 25 μm as measured in a suitable assay such as light scattering. In preferred embodiments the average particle size (Dv50 or median volume distribution) is determined by light scattering as described by USP <429> “Light Diffraction Measurements of Particle Size”.

23C. A method of treatment or prophylaxis of an autoimmune disease or unwanted inflammation condition, which optionally is an arthritis condition such as an osteoarthritis (primary or secondary osteoarthritis), rheumatoid arthritis, an arthritis associated with spondylitis such as ankylosing spondylitis, multiple sclerosis, Alzheimer's disease, tenosynovitis, a lupus condition such as systemic lupus erythematosis or discoid lupus erythematosis, tendinitis, bursitis, a lung inflammation condition such as asthma, emphysema, chronic obstructive pulmonary disease, lung fibrosis, cystic fibrosis, acute or adult respiratory distress syndrome, chronic bronchitis, acute bronchitis, bronchiolitis, bronchiolitis fibrosa obliterans, bronchiolitis obliterans with organizing pneumonia, using crystalline 3β-tetrol or a formulation or composition prepared from crystalline 3β-tetrol wherein the crystalline form is Form IIβ, Form IIIβ, Form IVβ, Form Vβ, Form VIβ, Form VIIβ, Form VIIIβ, Form IXβ or Form VIβ or a mixture thereof, optionally wherein Form Iβ 3β-tetrol is present as a minor component in comparison to the total crystalline content of 36-tetrol.

24C. The method of embodiment 23C comprising administering to the human or the rodent a treatment effective amount of 3β-tetrol. Such treatments include treatment with about 0.1 mg/day, about 1 mg/day or about 5 mg/day to about 40 mg/day or about 80 mg/day of 3β-tetrol.

25C. The method of embodiment 23C wherein the autoimmune or related disorder is ulcerative colitis, inflammatory bowel disease, Crohn's disease, psoriasis, actinic keratosis, arthritis, multiple sclerosis, optic neuritis or a dermatitis condition, optionally contact dermatitis, atopic dermatitis or exfoliative dermatitis.

26C. The method of any one of embodiments 1C-25C wherein the androst-5-ene-3β,7β,16α,17β-tetrol crystalline form is Form IIβ 3β-tetrol.

27C. The method of any one of embodiments 1C-25C wherein the androst-5-ene-3β,7β,16α,17β-tetrol crystalline form is Form IIIβ 3β-tetrol.

28C. The method of any one of embodiments 1C-25C wherein the androst-5-ene-3β,7β,16α,17β-tetrol crystalline form is Form IVβ 3β-tetrol.

29C. The method of any one of embodiments 1C-25C wherein the androst-5-ene-3β,7β,16α,17β-tetrol crystalline form is Form Vβ 3β-tetrol.

30C. The method of any one of embodiments 1C-25C wherein the androst-5-ene-3β,7β,16α,17β-tetrol crystalline form is Form VIβ 3β-tetrol.

31C. The method of any one of embodiments 1C-25C wherein the androst-5-ene-3β,7β,16α,17β-tetrol crystalline form is Form VIIβ 3β-tetrol.

32C. The method of any one of embodiments 1C-25C wherein the androst-5-ene-3β,7β,16α,17β-tetrol crystalline form is Form VIIIβ 3β-tetrol.

33C. The method of any one of embodiments 1C-25C wherein the androst-5-ene-3β,7β,16α,17β-tetrol crystalline form is Form IXβ 3β-tetrol.

34C. The method of any one of embodiments 1C-25C wherein the androst-5-ene-3β,7β,16α,17β-tetrol crystalline form is Form Xβ 3β-tetrol.

1D. A crystalline form of androst-5-ene-3β,7β,16α,17β-tetrol wherein the crystalline form is (1) not characterized by XRPD pattern having three or more peaks selected from the group consisting of 14.8±0.1, 15.9±0.1, 17.3±0.1, 19.2±0.1, 20.4±0.1 and 29.4±0.1 degree 2-theta that have a relative intensity of 10% or more relative to the most intense peak of the XRPD pattern

2D. A composition or formulation comprised of a crystalline form or forms of androst-5-ene-3β,7β,16α,17β-tetrol wherein the crystalline form is characterized by (1) XRPD pattern having peaks of 14.8±0.1, 15.9±0.1, 17.3±0.1, 19.2±0.1, 20.4±0.1 and 29.4±0.1 degree 2-theta and (2) a DTA thermogram, obtained with a temperature ramp of about 10° C./min, having an endotherm centered at about 204° C. and absent temperature transitions between 140° C. to 195° C. is present as a minor component in comparison to the total crystalline content of androst-5-ene-3β,7β,16α,17β-tetrol.

3D. The composition, formulation or crystalline form of embodiment 1D or 2D wherein the crystalline form that is present and is not a minor component is characterized by (1) XRPD pattern having peaks at about 16.1±0.1 degree 2-theta and three or more peaks selected form the group consisting of 7.6±0.1, 14.9±0.1, 25.4±0.1 and 29.6±0.1 degree 2-theta or XRPD pattern having five or more peaks selected from the group consisting of 7.6±0.1, 14.9±0.1, 15.4±0.1, 16.1±0.1, 17.3±0.1, 19.9±0.1, 25.4±0.1 and 29.6±0.1 degree 2-theta or (2) a solid Raman spectrum having three, four or more absorbances selected from the group consisting of 239, 445, 474, 987, 1057, 1091, 1128, 1275, 1329, 1344 and 1437 cm⁻¹ and optionally one or more absorbances selected from the group consisting of 2858, 2891 and 2964 cm⁻¹ or a solid Raman spectrum with five, six or more absorbances selected from the group consisting of 216, 239, 345, 386, 445, 474, 598, 661, 700, 779, 987, 1057, 1091, 1128, 1194, 1275, 1329, 1344 and 1437 cm⁻¹ or (3): (1) and (2).

4D. The composition, formulation or crystalline form of embodiment 1D or 2D wherein the crystalline form that is present and is not a minor component is characterized by (1) XRPD pattern having peaks at about 16.2±0.1 and 15.4±0.1 degree 2-theta and two or more peaks selected from the group consisting of 7.7±0.1, 14.8±0.1, 19.7±0.1, 20.8±0.1, 25.3±0.1 and 29.9±0.1 degree 2-theta or XRPD pattern having five or more peaks selected from the group consisting of 7.7±0.1, 14.8±0.1, 15.4±0.1, 16.2±0.1, 19.7±0.1, 20.8±0.1, 25.3±0.1 and 29.9±0.1 degree 2-theta or (2) a solid Raman spectrum having three, four or more absorbances selected from the group consisting of 235, 443, 474, 517, 536, 901, 985, 1009, 1045, 1055, 1090, 1099, 1172, 1279, 1329, 1342 and 1437 cm⁻¹ and optionally one or more absorbances selected from the group consisting of 2866, 2891, 2900 and 2966 cm⁻¹ or a solid Raman spectrum having five, six or more absorbances selected from the group consisting of 214, 235, 345, 386, 443, 474, 517, 536, 598, 661, 700, 779, 901, 985, 1009, 1045, 1055, 1090, 1099, 1126, 1172, 1194, 1279, 1329, 1342 and 1437 cm⁻¹ or (3): (1) and (2).

5D. The composition, formulation or crystalline form of embodiment 1D or 2D wherein the crystalline form that is present and is not a minor component is characterized by (1) XRPD pattern having two or more peaks selected from the group consisting of 14.6±0.1, 14.8±0.1, 15.9±0.1, 17.3±0.1 and 29.4±0.1 degree 2-theta and two or more peak selected from the group consisting of 7.4±0.1, 17.8±0.1, 19.2±0.1, 19.8±0.1, 20.2±0.1, 20.3±0.1, 21.1±0.1, 22.7±0.1, 24.4±0.1, 25.5±0.1 and 27.3±0.1 degree 2-theta, or XRPD pattern having five or more peaks selected from the group consisting of 7.4±0.1, 14.6±0.1, 14.8±0.1, 15.9±0.1, 17.3±0.1, 17.8±0.1, 19.2±0.1, 19.8±0.1, 20.2±0.1, 20.3±0.1, 21.1±0.1, 22.7±0.1, 24.4±0.1, 25.5±0.1, 27.3±0.1 and 29.4±0.1 degree 2-theta or (2) a solid Raman spectrum having three, four or more absorbances selected from the group consisting of 285, 297, 374, 443, 472, 536, 985, 1009, 1055, 1099, 1172, 1279, 1329, 1344 and 1439 cm⁻¹ and optionally one or more absorbances selected from the group consisting of 2868, 2893, 2902, 2937 and 2966 cm⁻¹ or a solid Raman spectrum with five, six or more absorbances selected from the group consisting of 187, 214, 285, 297, 345, 374, 386, 443, 472, 536, 598, 661, 700, 779, 985, 1009, 1055, 1099, 1126, 1172, 1194, 1279, 1329, 1344 and 1439 cm⁻¹ or (3): (1) and (2).

6D. The composition, formulation or crystalline form of embodiment 1D or 2D wherein the crystalline form that is present and is not a minor component is characterized by (1) XRPD pattern having two or more peaks selected from the group consisting of 13.1±0.1, 15.0±0.1, 15.4±0.1, 16.2±0.1, 17.0±0.1 and 19.9±0.1 degree 2-theta and two or more peak selected from the group consisting of 6.5±0.1, 7.7±0.1, 8.2±0.1, 15.0±0.1, 22.3±0.1 and 25.4±0.1 degree 2-theta or XRPD pattern having five or more peaks selected from the group consisting of 6.5±0.1, 7.7±0.1, 8.2±0.1, 9.7±0.1, 13.1±0.1, 14.8±0.1, 15.0±0.1, 15.4±0.1, 15.9±0.1, 16.2±0.1, 16.7±0.1, 17.0±0.1, 19.3±0.1, 19.6±0.1, 19.9±0.1, 20.8±0.1, 20.9±0.1, 21.1±0.1, 21.2±0.1, 21.9±0.1, 22.3±0.1, 23.1±0.1, 23.5±0.1, 23.9±0.1, 24.6±0.1, 25.3±0.1, 25.4±0.1 and 29.8±0.1 degree 2-theta or (2) a solid Raman spectrum having three or more absorbances selected from the group consisting of 243, 341, 384, 440, 447, 476, 519, 696, 901, 987, 1007, 1036, 1059, 1091, 1149, 1196, 1232, 1250, 1273, 1319, 1344, 1439 and 1462 cm⁻¹ and optionally one or more absorbances selected from the group consisting of 2854, 2871, 2891, 2937, 2954 and 2970 cm⁻¹ or a solid Raman spectrum having five or more absorbances selected from the group consisting of 216, 243, 341, 384, 440, 447, 476, 519, 598, 661, 696, 779, 847, 901, 987, 1007, 1036, 1059, 1091, 1126, 1149, 1174, 1196, 1232, 1250, 1273, 1319, 1344 and 1439 cm⁻¹ or (3): (1) and (2).

7D. The composition, formulation or crystalline form of embodiment 1D or 2D wherein the crystalline form that is present and is not a minor component is characterized (1) XRPD pattern having two or more peaks selected from the group consisting of 6.1±0.1, 15.4±0.1, 16.2±0.1 and 25.3±0.1 degree 2-theta and two or more peaks selected from the group consisting of 7.6±0.1, 8.1±0.1, 12.2±0.1, 18.2±0.1, 19.6±0.1, 19.9±0.1, 20.8±0.1 and 29.8±0.1 degree 2-theta or XRPD pattern having five or more peaks selected from the group consisting of 6.1±0.1, 7.6±0.1, 8.1±0.1, 12.2±0.1, 15.4±0.1, 16.2±0.1, 18.2±0.1, 19.6±0.1, 19.9±0.1, 20.8±0.1, 25.3±0.1 and 29.8±0.1 degree 2-theta or (2) a solid Raman spectrum having three or more absorbances selected from the group consisting of 239, 345, 386, 443, 474, 987, 1010, 1057, 1099, 1277, 1329, 1346 and 1439 cm⁻¹ and optionally one or more absorbances selected from the group consisting of 2856, 2870, 2893, 2902, 2939 and 2968 cm⁻¹ or a solid Raman spectrum having five or more absorbances selected from the group consisting of 216, 239, 345, 386, 443, 474, 598, 661, 700, 779, 987, 1010, 1057, 1099, 1126, 1174, 1194, 1277, 1329, 1346 and 1439 cm⁻¹ or (3): (1) and (2).

8D. The composition, formulation or crystalline form of embodiment 1D or 2D wherein the crystalline form that is present and is not a minor component is characterized by (1) XRPD pattern having three or more peaks selected from the group consisting of 14.8±0.1, 15.9±0.1 and 17.3±0.1 degree 2-theta one or more peaks selected from the group consisting of 7.4±0.1, 20.4±0.1 and 24.4±0.1 degree 2-theta or XRPD pattern having five or more peaks selected from the group consisting of 7.4±0.1, 14.6±0.1, 14.9±0.1, 15.9±0.1, 17.3±0.1, 17.9±0.1, 19.2±0.1, 19.8±0.1, 20.2±0.1, 20.4±0.1, 24.4±0.1, 25.6±0.1, 27.4±0.1 and 29.4±0.1 degree 2-theta or (2) a solid Raman spectrum having three or more absorbances selected from the group consisting of 345, 386, 443, 472, 536, 598, 661, 700, 779, 901, 985, 1009, 1057, 1099, 1126, 1174, 1194, 1277, 1327, 1346 and 1437 cm⁻¹, and optionally one or more absorbances selected from the group consisting of 2835, 2856, 2868, 2902, 2931 and 2937 cm⁻¹ or a solid Raman spectrum having five or more absorbances selected from the group consisting of 216, 345, 386, 443, 472, 536, 598, 661, 700, 779, 901, 985, 1009, 1057, 1099, 1126, 1174, 1194, 1277, 1327, 1346, and 1437 cm⁻¹ or (3): (1) and (2).

9D. The composition, formulation or crystalline form of embodiment 1D or 2D wherein the crystalline form that is present and is not a minor component is characterized by (1) XRPD pattern having three or more peaks selected from the group consisting of 6.1±0.1, 12.1±0.1, 15.8±0.1, 16.8±0.1, 18.2±0.1 and 21.8±0.1 degree 2-theta and one or more peaks selected from the group consisting of 13.0±0.1, 13.6±0.1, 14.0±0.1, 18.6±0.1 and 20.8±0.1 degree 2-theta or XRPD pattern having five or more peaks selected from the group consisting of 6.1±0.1, 8.1±0.1, 9.9±0.1, 12.1±0.1, 13.0±0.1, 13.6±0.1, 14.0±0.1, 15.8±0.1, 16.8±0.1, 18.2±0.1, 18.6±0.1, 19.8±0.1, 20.8±0.1, 21.8±0.1, 24.3±0.1 and 29.8±0.1 degree 2-theta.

1E. The crystalline androst-5-ene-3β,7β,16α,17β-tetrol of embodiment 1 wherein crystalline androst-5-ene-3β,7β,16α,17β-tetrol is Form IIβ 3β-tetrol characterized by DTA thermogram, obtained with a temperature ramp of 10° C./min, having a prominent endotherm at about 252° C., an endotherm at 239° C. and an exotherm at 242° C.

2E. The crystalline androst-5-ene-3β,7β,16α,17β-tetrol of embodiment 1E wherein the 239° C. endotherm has an onset temperature of 235° C.

3E. The crystalline androst-5-ene-3β,7β,16α,17β-tetrol of embodiment 1E or 2E wherein crystalline androst-5-ene-3β,7β,16α,17β-tetrol is further characterized by a TGA thermogram, obtained with a temperature ramp of 10° C./min, having 5% wt loss from between 60° C. to 140° C.

4E. The crystalline androst-5-ene-3β,7β,16α,17β-tetrol of embodiment 3E wherein the TGA % wt loss is associated with a broad endotherm in DTA centered at 102° C.

5E. The crystalline androst-5-ene-3β,7β,16α,17β-tetrol of embodiment 1 wherein crystalline androst-5-ene-3β,7β,16α,17β-tetrol is Form IIIβ 3β-tetrol characterized by (1) XRPD pattern having three or more peaks selected from the group consisting of 7.6, 14.9, 25.4 and 29.6 degree 2-theta and/or one or more peaks selected from the group consisting of 15.4, 16.1, 17.3 and 19.9 degree 2-theta or (2) DTA thermogram, obtained with a temperature ramp of 10° C./min, having a prominent endotherm at centered at 200° C. with an onset temperature of 191° C. or (3): (1) and (2).

6E. The crystalline androst-5-ene-3β,7β,16α,17β-tetrol of embodiment 5E wherein the prominent DTA endotherm has a shoulder at 210° C.

7E. The crystalline androst-5-ene-3β,7β,16α,17β-tetrol of embodiment 1 wherein crystalline androst-5-ene-3β,7β,16α,17β-tetrol is Form IIIβ 3βtetrol characterized by solid state Raman spectrum having absorbances at 1275, 1329, 1344 and 1437 cm⁻¹ and/or four or more absorbances selected from the group consisting of 445, 474, 987, 1057, 1091 and 1128 cm⁻¹.

8E. The crystalline androst-5-ene-3β,7β,16α,17β-tetrol of embodiment 5E or 6E wherein crystalline androst-5-ene-3β,7β,16α,17β-tetrol is further characterized by solid state Raman spectrum having absorbances at 1275, 1329, 1344 and 1437 cm⁻¹ and/or four or more absorbances selected from the group consisting of 445, 474, 987, 1057, 1091 and 1128 cm⁻¹.

9E. The crystalline androst-5-ene-3β,7β,16α,17β-tetrol of any one of embodiments 5E-8E wherein crystalline androst-5-ene-3β,7β,16α,17β-tetrol is further characterized by TGA thermogram, obtained with a temperature ramp of 10° C./min, having 5% wt loss from between 60° C. to 140° C.

10E. The crystalline androst-5-ene-3β,7β,16α,17β-tetrol of embodiment 9E wherein the TGA thermogram % wt loss between 60° C. to 140° C. is associated with a broad endotherm in DTA thermogram centered at 103° C.

11E. The crystalline androst-5-ene-3β,7β,16α,17β-tetrol of any one of embodiments 5E-10E wherein the crystalline androst-5-ene-3β,7β,16α,17β-tetrol is further characterized by TGA thermogram % wt loss of 5-10% or more that is associated with the prominent DTA endotherm centered at 200° C.

12E. The crystalline androst-5-ene-3β,7β,16α,17β-tetrol of embodiment 1 wherein crystalline androst-5-ene-3β,7β,16α,17β-tetrol is Form IVβ 3β-tetrol characterized by (1) XRPD pattern having three or more peaks selected from the group consisting of 7.7, 15.4, 16.2 and 25.3 degree 2-theta and one or more peaks selected from the group consisting of 14.8, 19.7, 20.8 and 29.9 degree 2-theta or (2) DTA thermogram, obtained with a temperature ramp of 10° C./min, having a prominent endotherm centered at 233° C. or (3): (1) and (2).

13E. The crystalline androst-5-ene-3β,7β,16α,17β-tetrol of embodiment 12E wherein the prominent DTA 233° C. endotherm has an onset temperature of 228° C.

14E. The crystalline androst-5-ene-3β,7β,16α,17β-tetrol of embodiment 12E or 13E wherein the DTA thermogram additionally contains a weak, broad endotherm at 197° C.

15E. The crystalline androst-5-ene-3β,7β,16α,17β-tetrol of embodiment 14E wherein the DTA 197° C. endotherm has an onset temperature of 189° C.

16E. The crystalline androst-5-ene-3β,7β,16α,17β-tetrol of any one of embodiments 12E-15E wherein the DTA has a broad endotherm centered at 99° C.

17E. The crystalline androst-5-ene-3β,7β,16α,17β-tetrol of any one of embodiments 11E to 16E wherein crystalline androst-5-ene-3β,7β,16α,17β-tetrol is further characterized by TGA thermogram, obtained with a temperature ramp of 10° C./min, having about 5% wt loss from between about 60° C. to about 140° C.

18E. The crystalline androst-5-ene-3β,7β,16α,17β-tetrol of embodiment 1 wherein crystalline androst-5-ene-3β,7β,16α,17β-tetrol is Form IVβ 3β-tetrol characterized by solid state Raman spectrum having absorbances at 1279, 1329, 1342 and 1437 cm⁻¹ and/or four or more absorbances selected from the group consisting of 443, 474, 517, 536, 901, 985, 1009, 1045, 1090, 1099 and 1172 cm⁻¹.

19E. The crystalline androst-5-ene-3β,7β,16α,17β-tetrol of any one of embodiments 12E to 17E wherein crystalline androst-5-ene-3β,7β,16α,17β-tetrol is further characterized by solid state Raman spectrum having absorbances at 1279, 1329, 1342 and 1437 cm⁻¹ and/or four or more absorbances selected from the group consisting of 443, 474, 517, 536, 901, 985, 1009, 1045, 1090, 1099 and 1172 cm⁻¹.

20E. The crystalline androst-5-ene-3β,7β,16α,17β-tetrol of embodiment 1 wherein crystalline androst-5-ene-3β,7β,16α,17β-tetrol is Form Vβ 3β-tetrol characterized by (1) XRPD pattern having three or more peaks selected from the group consisting of 7.4, 14.8, 15.9, 17.3, 19.2, 20.2, 24.4 and 29.4 degree 2-theta and one or more peaks selected from the group consisting of 14.6, 17.8, 19.8, 20.3, 21.1, 22.7, 25.5 and 27.3 degree 2-theta or (2) DTA thermogram, obtained with a temperature ramp of 10° C./min, having a prominent endotherm at about 230° C. or (3): (1) and (2).

21E. The crystalline androst-5-ene-3β,7β,16α,17β-tetrol of embodiment 20E wherein the prominent DTA endotherm has an onset temperature of about 223° C.

22E. The crystalline androst-5-ene-3β,7β,16α,17β-tetrol of embodiment 20E or 21E wherein the DTA thermogram has a weak exotherm at about 188° C.

23E. The crystalline androst-5-ene-3β,7β,16α,17β-tetrol of embodiment 20E, 21E or 22E wherein crystalline androst-5-ene-3β,7β,16α,17β-tetrol is further characterized by a TGA thermogram, obtained with a temperature ramp of 10° C./min, having 5% wt loss from between 60° C. to 140° C.

24E. The crystalline androst-5-ene-3β,7β,16α,17β-tetrol of 23E wherein the TGA % wt loss between 60° C. to 140° C. is associated with a broad endotherm in DTA centered at 102° C.

25E. The crystalline androst-5-ene-3β,7β,16α,17β-tetrol of embodiment 1 wherein crystalline androst-5-ene-3β,7β,16α,17β-tetrol is Form Vβ 3β-tetrol characterized by solid state Raman spectrum having absorbances at 1279, 1329, 1344 and 1439 cm⁻¹ or four or more absorbances selected from the group consisting of 443, 472, 536, 985, 1009, 1055, 1099 and 1172 cm⁻¹.

26E. The crystalline androst-5-ene-3β,7β,16α,17β-tetrol of any one of embodiments 20E-24E wherein crystalline androst-5-ene-3β,7β,16α,17β-tetrol is further characterized by solid state Raman spectrum having absorbances at 1279, 1329, 1344 and 1439 cm⁻¹ or four or more absorbances selected from the group consisting of 443, 472, 536, 985, 1009, 1055, 1099 and 1172 cm⁻¹.

27E. The crystalline androst-5-ene-3β,7β,16α,17β-tetrol of embodiment 1 wherein crystalline androst-5-ene-3β,7β,16α,17β-tetrol is Form VIβ 3β-tetrol characterized by (1) XRPD pattern having three or more peaks selected from the group consisting 6.5, 7.7, 8.2, 13.1, 15.0, 15.4, 16.2, 17.0, 19.9, 22.3 and 25.4 degree 2-theta and one or more peaks selected from the group consisting of 9.7, 14.8, 15.9, 16.7, 19.3, 19.6, 20.8, 20.9, 21.1, 21.2, 21.9, 23.1, 23.5, 23.9, 24.6, 25.3 and 29.8 degree 2-theta or (2) DTA thermogram, obtained with a temperature ramp of 10° C./min, having a prominent endotherm at about 233° C., and no endotherm between 140° C. to 200° C. or (3): (1) and (2).

28E. The crystalline androst-5-ene-3β,7β,16α,17β-tetrol of embodiment 27E wherein the prominent DTA endotherm an onset temperature of about 226° C.

29E. The crystalline androst-5-ene-3β,7β,16α,17β-tetrol of embodiment 27E or 28E wherein the crystalline androst-5-ene-3β,7β,16α,17β-tetrol is further characterized by TGA thermogram, obtained with a temperature ramp of 10° C./min, having about 10% wt loss from between 60° C. to 140° C.

30E. The crystalline androst-5-ene-3β,7β,16α,17β-tetrol of embodiment 29E wherein the TGA wt % loss between 60° C. to 140° C. is associated with a broad endotherm in DTA centered at 111° C.

31E. The crystalline androst-5-ene-3β,7β,16α,17β-tetrol of embodiment 1 wherein crystalline androst-5-ene-3β,7β,16α,17β-tetrol is Form VIβ 3β-tetrol characterized by solid state Raman spectrum having four or more absorbances selected from the group consisting of 1196, 1232, 1250, 1273, 1319, 1344, 1439 and 1462 cm⁻¹ and/or four or more absorbances selected from the group consisting of 440, 447, 476, 519, 696, 901, 987, 1007, 1036, 1059 and 1091 cm⁻¹.

32E. The crystalline androst-5-ene-3β,7β,16α,17β-tetrol of any one of embodiments 27E to 30E wherein crystalline androst-5-ene-3β,7β,16α,17β-tetrol is further characterized by solid state Raman spectrum having four or more absorbances selected from the group consisting of 1196, 1232, 1250, 1273, 1319, 1344, 1439 and 1462 cm⁻¹ and/or four or more absorbances selected from the group consisting of 440, 447, 476, 519, 696, 901, 987, 1007, 1036, 1059 and 1091 cm⁻¹.

33E. The crystalline androst-5-ene-3β,7β,16α,17β-tetrol of embodiment 1 wherein crystalline androst-5-ene-3β,7β,16α,17β-tetrol is Form VIIIβ 3β-tetrol characterized by (1) XRPD pattern having three or more peaks selected from the group consisting of 6.1, 12.2, 16.2 and 25.3 degree 2-theta and one or more peaks selected from the group consisting of 7.6, 8.1, 15.4, 18.2, 19.6, 19.9, 20.8 and 29.8 degree 2-theta or (2) DTA thermogram, obtained with a temperature ramp of 10° C./min, having a prominent endotherm at 233° C. or (3): (1) and (2).

34E. The crystalline androst-5-ene-3β,7β,16α,17β-tetrol of embodiment 23E wherein the 233° C. prominent endotherm has an onset temperature of about 225° C.

35E. The crystalline androst-5-ene-3β,7β,16α,17β-tetrol of embodiment 33E or 34E wherein the DTA thermogram additionally has a broad endotherm centered at 178° C.

36E. The crystalline androst-5-ene-3β,7β,16α,17β-tetrol of embodiment 35E wherein the 178° C. broad endotherm has an onset temperature of 163° C.

37E. The crystalline androst-5-ene-3β,7β,16α,17β-tetrol of embodiment 1 wherein crystalline androst-5-ene-3β,7β,16α,17β-tetrol is Form VIIβ 3β-tetrol characterized by DTA thermogram, obtained with a temperature ramp of 10° C./min, having a prominent endotherm at 252° C. and no thermal transitions from between 60° C. to the onset temperature of that prominent endotherm.

38E. The crystalline androst-5-ene-3β,7β,16α,17β-tetrol of embodiment 37E wherein the onset temperature is 239° C.

39E. The crystalline androst-5-ene-3β,7β,16α,17β-tetrol of embodiment 1 wherein crystalline androst-5-ene-3β,7β,16α,17β-tetrol is Form VIIIβ 3β-tetrol characterized by (1) XRPD pattern having three or more peaks selected from the group consisting of 6.1, 12.2, 16.2 and 25.3 degree 2-theta and one or more peaks selected from the group consisting of 7.6, 8.1, 15.4, 18.2, 19.6, 19.9, 20.8 and 29.8 degree 2-theta or (2) DTA thermogram, obtained with a temperature ramp of 10° C./min, having a prominent endotherm centered at 233° C. or (3): (1) and (2).

40E. The crystalline androst-5-ene-3β,7β,16α,17β-tetrol of embodiment 39E wherein the prominent 233° C. endothem has an onset temperature of 225° C.

41E. The crystalline androst-5-ene-3β,7β,16α,17β-tetrol of embodiment 39E or 40E wherein the DTA thermogram additionally has a broad endotherm centered at 178° C.

42E. The crystalline androst-5-ene-3β,7β,16α,17β-tetrol of embodiment 41E wherein the 178° C. broad endotherm has an onset temperature of 163° C.

43E. The crystalline androst-5-ene-3β,7β,16α,17β-tetrol of any one of embodiments 39E-42E wherein crystalline androst-5-ene-3β,7β,16α,17β-tetrol is further characterized by TGA thermogram, obtained with a temperature ramp of 10° C./min, having negligible % wt loss from between 60° C. to 140° C.

44E. The crystalline androst-5-ene-3β,7β,16α,17β-tetrol of embodiment 43E wherein the TGA wt % loss, if any, between 60° C. to 140° C. is associated with a broad endotherm in DTA thermogram centered at 85° C. of variable intensity.

45E. The crystalline androst-5-ene-3β,7β,16α,17β-tetrol of embodiment 1 wherein crystalline androst-5-ene-3β,7β,16α,17β-tetrol is Form VIIβ 3β-tetrol characterized by solid state Raman spectrum having absorbances at 1277, 1329, 1346 and 1439 cm⁻¹ and/or four or more absorbances selected from the group consisting of 443, 474, 987, 1010, 1057 and 1099 cm⁻¹.

46E. The crystalline androst-5-ene-3β,7β,16α,17β-tetrol of any one of embodiments 39E-44E wherein crystalline androst-5-ene-3β,7β,16α,17β-tetrol is further characterized by solid state Raman spectrum having absorbances at 1277, 1329, 1346 and 1439 cm⁻¹ and/or four or more absorbances selected from the group consisting of 443, 474, 987, 1010, 1057 and 1099 cm⁻¹.

47E. The crystalline androst-5-ene-3β,7β,16α,17β-tetrol of embodiment 1 wherein crystalline androst-5-ene-3β,7β,16α,17β-tetrol is Form IXβ 3β-tetrol characterized by (1) XRPD pattern having three or more peaks selected from the group consisting of 7.4, 14.9, 15.9, 17.3, 20.4 and 24.4 degree 2-theta and one or more peaks selected from the group consisting of about 14.6, 17.9, 19.2, 19.8, 20.2, 25.6, 27.4 and 29.4 degree 2-theta or (2) DTA thermogram, obtained with a temperature ramp of 10° C./min, having a prominent endotherm centered at 180° C. or (3): (1) and (2).

48E. The crystalline androst-5-ene-3β,7β,16α,17β-tetrol of embodiment 47E wherein the prominent 180° C. DTA endotherm has an onset temperature of 165° C.

49E. The crystalline androst-5-ene-3β,7β,16α,17β-tetrol of embodiment 47E or 48E wherein the prominent 180° C. DTA endotherm has a shoulder at 187° C.

50E. The crystalline androst-5-ene-3β,7β,16α,17β-tetrol of embodiment 47E, 48E or 49E wherein crystalline androst-5-ene-3β,7β,16α,17β-tetrol is further characterized by TGA thermogram, obtained with a temperature ramp of 10° C./min, having negligible % wt loss from between 60° C. to 140° C.

51E. The crystalline androst-5-ene-3β,7β,16α,17β-tetrol of embodiment 50E wherein the TGA wt % loss, if any, between 60° C. to 140° C. is associated with a broad DTA endotherm centered at 81° C. of variable intensity.

52E. The crystalline androst-5-ene-3β,7β,16α,17β-tetrol of any one of embodiments 47E-51E wherein crystalline androst-5-ene-3β,7β,16α,17β-tetrol is further characterized by TGA thermogram, obtained with a temperature ramp of 10° C./min, having between 5-10 wt % loss or more associated with the prominent DTA 180° C. endotherm.

53E. The crystalline androst-5-ene-3β,7β,16α,17β-tetrol of any one of embodiments 47E-52E wherein crystalline androst-5-ene-3β,7β,16α,17β-tetrol is further characterized by TGA thermogram, obtained with a temperature ramp of 10° C./min, having between 5-10 wt % loss or more associated with a very broad endotherm between 220° C. to 260° C.

54E. The crystalline androst-5-ene-3β,7β,16α,17β-tetrol of embodiment 1 wherein crystalline androst-5-ene-3β,7β,16α,17β-tetrol is Form IXβ 3β-tetrol characterized by solid state Raman spectrum having absorbances at 1277, 1327, 1346 and 1437 cm⁻¹ and/or four or more absorbances selected from the group consisting of 443, 472, 536, 598, 901, 985, 1009, 1057 and 1099 cm⁻¹.

55E. The crystalline androst-5-ene-3β,7β,16α,17β-tetrol of any one of embodiments 47-53 wherein the crystalline androst-5-ene-3β,7β,16α,17β-tetrol is characterized by solid state Raman spectrum having absorbances at 1277, 1327, 1346 and 1437 cm⁻¹ and/or four or more absorbances selected from the group consisting of 443, 472, 536, 598, 901, 985, 1009, 1057 and 1099 cm⁻¹.

56E. The crystalline androst-5-ene-3β,7β,16α,17β-tetrol of embodiment 1 wherein crystalline androst-5-ene-3β,7β,16α,17β-tetrol is Form Xβ 3β-tetrol characterized by (1) XRPD pattern having three or more XRPD peaks selected from the group consisting of 6.1, 12.1, 13.0, 13.6, 14.0, 15.8, 18.2 and 18.6 degree 2-theta and one or more peaks selected from the group consisting of 8.1, 9.9, 16.8, 19.8, 20.8, 21.8, 24.3 and 29.8 degree 2-theta or (2) DTA thermogram, obtained with a temperature ramp of 10° C./min, having a prominent endotherm at 204° C. or (3): (1) and (2).

57E. The crystalline androst-5-ene-3β,7β,16α,17β-tetrol of embodiment 56E wherein the prominent DTA 180° C. endotherm has an onset temperature of 190° C.

58E. The crystalline androst-5-ene-3β,7β,16α,17β-tetrol of embodiment 56E or 57E wherein the prominent DTA 180° C. endotherm has a shoulder at 217° C.

59E. The crystalline androst-5-ene-3β,7β,16α,17β-tetrol of embodiment 56E, 57E or 58E wherein crystalline androst-5-ene-3β,7β,16α,17β-tetrol is further characterized by TGA thermogram, obtained with a temperature ramp of 10° C./min, having negligible % wt loss from between 60° C. to 140° C.

60E. The crystalline androst-5-ene-3β,7β,16α,17β-tetrol of embodiment 59E wherein the TGA wt % loss, if present, between 60° C. to 140° C. is associated with a broad endotherm in DTA centered at 81° C. of variable intensity.

61E. The crystalline androst-5-ene-3β,7β,16α,17β-tetrol of any one of embodiments 56E-60E wherein crystalline androst-5-ene-3β,7β,16α,17β-tetrol is further characterized by TGA thermogram, obtained with a temperature ramp of 10° C./min, having between 5-10 wt % loss or more associated with the prominent 204° C. DTA endotherm.

1F. Crystalline androst-5-ene-3β,7β,16α,17β-tetrol of embodiment 1 wherein crystalline androst-5-ene-3β,7β,16α,17β-tetrol is Form IIβ 3β-tetrol characterized by DTA thermogram, obtained with a temperature ramp of 10° C./min, having (1) a prominent endotherm centered at 252° C., (2) an endotherm centered at 239° C. or (3) an exotherm centered at 242° C. or (4): (1) and (2), or (1) and (3), or (2) and (3), or (1), (2) and (3).

2F. The crystalline androst-5-ene-3β,7β,16α,17β-tetrol of embodiment 1 wherein the DTA 242° C. endotherm has an onset temperature of 235° C.

3F. The crystalline androst-5-ene-3β,7β,16α,17β-tetrol of embodiment 1F or 2F wherein the DTA thermogram has a broad endotherm centered at 102° C.

4F. The crystalline androst-5-ene-3β,7β,16α,17β-tetrol of embodiment 1F, 2F or 3F wherein Form IIβ 3β-tetrol is further characterized by a TGA thermogram, obtained with a temperature ramp of 10° C./min, having 5% wt loss from between 60° C. to 140° C. associated with the DTA 102° C. endotherm.

5F. Crystalline androst-5-ene-3β,7β,16α,17β-tetrol of embodiment 1 wherein crystalline androst-5-ene-3β,7β,16α,17β-tetrol is Form IIIβ 3βtetrol characterized by (1) XRPD pattern having three or more peaks selected from the group consisting of 7.6, 14.9, 25.4 and 29.6 degree 2-theta and one or more peaks selected from the group consisting of 15.4, 16.1, 17.3 and 19.9 degree 2-theta or (2) solid state Raman spectrum with absorbances at 1275, 1329, 1344 and 1437 cm⁻¹ and one or more absorbances selected from the group consisting of 445, 474, 987, 1057, 1091 and 1128 cm⁻¹ or (3): (1) and (2).

6F. The crystalline androst-5-ene-3β,7β,16α,17β-tetrol of embodiment 5F wherein Form IIIβ 3βtetrol is further characterized by (1) DTA thermogram, obtained with a temperature ramp of 10° C./min having a prominent endotherm centered at 200° C.

7F. The crystalline androst-5-ene-3β,7β,16α,17β-tetrol of embodiment 6F wherein the DTA 200° C. endotherm has (1) an onset temperature of about 191° C. or (2) a shoulder at 210° C. or (3): (1) and (2).

8F. The crystalline androst-5-ene-3β,7β,16α,17β-tetrol of embodiment 6F or 7F wherein the DTA thermogram has a broad endotherm centered at 103° C.

9F. The crystalline androst-5-ene-3β,7β,16α,17β-tetrol of embodiment 5F, 6F, 7F or 8F wherein Form IIIβ 3βtetrol is further characterized by TGA thermogram, obtained with a temperature ramp of 10° C./min, having (1) 5% wt loss from between 60° C. to 140° C. associated with the DTA 103° C. endotherm or (2) between 5%-10% wt loss or more associated with the DTA 200° C. endotherm or (3): (1) and (2).

10F. The crystalline androst-5-ene-3β,7β,16α,17β-tetrol of embodiment 1 wherein crystalline androst-5-ene-3β,7β,16α,17β-tetrol is Form IVβ 3β-tetrol characterized by (1) XRPD pattern having three or more peaks selected from the group consisting of 7.7, 15.4, 16.2 and 25.3 degree 2-theta and one or more peaks selected from the group consisting of about 14.8, 19.7, 20.8 and 29.9 degree 2-theta or (2) solid state Raman spectrum with absorbances at 1279, 1329, 1342 and 1437 cm⁻¹ and one or more absorbances selected from the group consisting of 443, 474, 517, 536, 901, 985, 1009, 1045, 1090, 1099 and 1172 cm⁻¹ or (3): (1) and (2).

11F. The crystalline androst-5-ene-3β,7β,16α,17β-tetrol of embodiment 10F wherein crystalline androst-5-ene-3β,7β,16α,17β-tetrol is further characterized by DTA thermogram, obtained with a temperature ramp of 10° C./min, having (1) a prominent endotherm centered at 233° C., (2) a weak, broad endotherm centered at 197° C. or (3): (1) and (2).

12F. The crystalline androst-5-ene-3β,7β,16α,17β-tetrol of embodiment 11F wherein the DTA 233° C. endotherm has an onset temperature of 228° C.

13F. The crystalline androst-5-ene-3β,7β,16α,17β-tetrol of embodiment 11F or 12F wherein the DTA 197° C. endotherm has an onset temperature of 189° C.

14F. The crystalline androst-5-ene-3β,7β,16α,17β-tetrol of embodiment 11F, 12F or 13F wherein the DTA thermogram has a broad endotherm centered at 99° C.

15F. The crystalline androst-5-ene-3β,7β,16α,17β-tetrol of embodiment 10F, 11F 12F, 13F or 14F wherein Form IVβ 3β-tetrol is further characterized by TGA thermogram, obtained with a temperature ramp of 10° C./min, having 5% wt loss from between 60° C. to 140° C. associated with the DTA 99° C. endotherm.

16F. Crystalline androst-5-ene-3β,7β,16α,17β-tetrol of embodiment 1 wherein crystalline androst-5-ene-3β,7β,16α,17β-tetrol is Form Vβ 3β-tetrol characterized by (1) XRPD pattern having three or more peaks selected from the group consisting of 7.4, 14.8, 15.9, 17.3, 19.2, 20.2, 24.4 and 29.4 degree 2-theta and one or more peaks selected from the group consisting of 14.6, 17.8, 19.8, 20.3, 21.1, 22.7, 25.5 and 27.3 degree 2-theta or (2) solid state Raman spectrum with absorbances at 1279, 1329, 1344 and 1439 cm⁻¹ and one or more absorbances selected from the group consisting of 443, 472, 536, 985, 1009, 1055, 1099 and 1172 cm⁻¹ or (1) and (2).

17F. The crystalline androst-5-ene-3β,7β,16α,17β-tetrol of embodiment 16F wherein Form Vβ 3β-tetrol is further characterized by (1) DTA thermogram, obtained with a temperature ramp of 10° C./min, having (1) a prominent endotherm centered at 230° C. or (2) a weak exotherm at 188° C. or (3): (1) and (2).

18F. The crystalline androst-5-ene-3β,7β,16α,17β-tetrol of embodiment 8F wherein the DTA 230° C. endotherm has an onset temperature of about 223° C.

19F. The crystalline androst-5-ene-3β,7β,16α,17β-tetrol of embodiment 17F or 18F wherein the DTA thermogram has a broad endotherm centered at 102° C.

20F. The crystalline androst-5-ene-3β,7β,16α,17β-tetrol of embodiment 16F, 17F, 18F or 19F wherein Form Vβ 3β-tetrol is further characterized by TGA thermogram, obtained with a temperature ramp of 10° C./min, having 5% wt loss from between 60° C. to 140° C. associated with the DTA 102° C. endotherm.

21F. The crystalline androst-5-ene-3β,7β,16α,17β-tetrol of embodiment 1 wherein crystalline androst-5-ene-3β,7β,16α,17β-tetrol is Form VIβ 3β-tetrol characterized by (1) XRPD pattern having three or more peaks selected from the group consisting 6.5, 7.7, 8.2, 13.1, 15.0, 15.4, 16.2, 17.0, 19.9, 22.3 and 25.4 degree 2-theta and one or more peaks selected from the group consisting of 9.7, 14.8, 15.9, 16.7, 19.3, 19.6, 20.8, 20.9, 21.1, 21.2, 21.9, 23.1, 23.5, 23.9, 24.6, 25.3 and 29.8 degree 2-theta or (2) solid state Raman spectrum with four or more absorbances selected from the group consisting of 1196, 1232, 1250, 1273, 1319, 1344, 1439 and 1462 cm⁻¹ and one or more absorbances selected from the group consisting of 440, 447, 476, 519, 696, 901, 987, 1007, 1036, 1059 and 1091 cm⁻¹ or (3): (1) and (2).

22F. The crystalline androst-5-ene-3β,7β,16α,17β-tetrol of embodiment 21F wherein Form VIβ 3β-tetrol is further characterized by DTA thermogram, obtained with a temperature ramp of 10° C./min, having (1) a prominent endotherm centered at 233° C. and no endotherm between 140° C. to 200° C. or (2) a broad endotherm centered at 111° C. or (3): (1) and (2).

23F. The crystalline androst-5-ene-3β,7β,16α,17β-tetrol of embodiment 22F wherein the DTA 233° C. endotherm has an onset temperature of 226° C.

24F. The crystalline androst-5-ene-3β,7β,16α,17β-tetrol of embodiment 21F, 22F or 23F wherein Form VIβ 3β-tetrol is further characterized by TGA thermogram, obtained with a temperature ramp of 10° C./min, having 10% wt loss from between 60° C. to 140° C. associated with the DTA 111° C. endotherm or (3): (1) and (2).

25F. Crystalline androst-5-ene-3β,7β,16α,17β-tetrol of embodiment 1 wherein crystalline androst-5-ene-3β,7β,16α,17β-tetrol is Form VIIβ 3β-tetrol characterized by DTA thermogram, obtained with a temperature ramp of 10° C./min, having a prominent endotherm centered at 252° C. and no thermal transitions from between 60° C. to the onset temperature of the DTA 252° C. endotherm.

26F. The crystalline androst-5-ene-3β,7β,16α,17β-tetrol of embodiment 25F wherein the DTA 252° C. endotherm has an onset temperature of about 239° C.

27F. Crystalline androst-5-ene-3β,7β,16α,17β-tetrol of embodiment 1 wherein crystalline androst-5-ene-3β,7β,16α,17β-tetrol is Form VIIIβ 3β-tetrol characterized by (1) XRPD pattern having three or more peaks selected from the group consisting of about 6.1, 12.2, 16.2 and 25.3 degree 2-theta and one or more peaks selected from the group consisting of 7.6, 8.1, 15.4, 18.2, 19.6, 19.9, 20.8 and 29.8 degree 2-theta or (2) solid state Raman spectrum with absorbances at 1277, 1329, 1346 and 1439 cm⁻¹ and one four or more absorbances selected from the group consisting of 443, 474, 987, 1010, 1057 and 1099 cm⁻¹ or (3): (1) and (2).

28F. The crystalline androst-5-ene-3β,7β,16α,17β-tetrol of embodiment 27F wherein Form VIIIβ 3β-tetrol is further characterized by DTA thermogram, obtained with a temperature ramp of 10° C./min, having (1) a prominent endotherm centered at about 239° C. or (2) a broad endotherm centered at about 178° C. or (3): (1) and (2).

29F. The crystalline androst-5-ene-3β,7β,16α,17β-tetrol of embodiment 27F wherein the DTA 233° C. endotherm has an onset temperature of about 225° C.

30F. The crystalline androst-5-ene-3β,7β,16α,17β-tetrol of embodiment 28F or 29F wherein the DTA 178° C. endotherm has an onset temperature of 163° C.

31F. The crystalline androst-5-ene-3β,7β,16α,17β-tetrol of embodiment 28F, 29F or 30F wherein the DTA thermogram has a broad endotherm in DTA centered at 85° C. of variable intensity.

32F. The crystalline androst-5-ene-3β,7β,16α,17β-tetrol of any one of embodiments 27F to 31F wherein Form VIIIβ 3β-tetrol is further characterized by TGA thermogram, obtained with a temperature ramp of 10° C./min, having negligible % wt loss from between about 60° C. to about 140° C.

33F. The crystalline androst-5-ene-3β,7β,16α,17β-tetrol of embodiment 1 wherein crystalline androst-5-ene-3β,7β,16α,17β-tetrol is Form IXβ 3β-tetrol characterized by (1) XRPD pattern having three or more peaks selected from the group consisting of about 7.4, 14.9, 15.9, 17.3, 20.4 and 24.4 degree 2-theta and one or more peaks selected from the group consisting of about 14.6, 17.9, 19.2, 19.8, 20.2, 25.6, 27.4 and 29.4 degree 2-theta or (2) solid state Raman spectrum with absorbances at 1277, 1329, 1346 and 1439 cm⁻¹ and one or more absorbances selected from the group consisting of 443, 472, 536, 598, 901, 985, 1009, 1057 and 1099 cm⁻¹ or (3): (1) and (2).

34F. The crystalline androst-5-ene-3β,7β,16α,17β-tetrol of claim 33F wherein Form IXβ 3β-tetrol is further characterized by (1) DTA thermogram, obtained with a temperature ramp of 10° C./min, having a prominent endotherm centered at 180° C.

35F. The crystalline androst-5-ene-3β,7β,16α,17β-tetrol of embodiment 34F wherein the DTA 180° C. endotherm has (1) an onset temperature of about 165° C. or (2) a shoulder at 187° C. or (3): (1) and (2).

36F. The crystalline androst-5-ene-3β,7β,16α,17β-tetrol of embodiment 34F or 35F wherein the DTA thermograph has with a very broad DTA endotherm between 220° C. to 260° C.

37F. The crystalline androst-5-ene-3β,7β,16α,17β-tetrol of embodiment 33F, 34F, 35F or 36F wherein Form IXβ is further characterized by TGA thermogram, obtained with a temperature ramp of 10° C./min, having (1) between 5-10% wt loss or more associated with the DTA 180° C. endotherm or (2) between 5% to 10% wt loss or more associated with the broad 220° C. to 260° C. DTA endotherm or (3): (1) and (2).

38F. Crystalline androst-5-ene-3β,7β,16α,17β-tetrol of embodiment 1 wherein crystalline androst-5-ene-3β,7β,16α,17β-tetrol is Form Xβ 3β-tetrol characterized by XRPD pattern having three or more XRPD peaks selected from the group consisting of 6.1, 12.1, 13.0, 13.6, 14.0, 15.8, 18.2 and 18.6 degree 2-theta and one or more peaks selected from the group consisting of 8.1, 9.9, 16.8, 19.8, 20.8, 21.8, 24.3 and 29.8 degree 2-theta.

39F. The crystalline androst-5-ene-3β,7β,16α,17β-tetrol of embodiment 38F wherein Form Xβ 3β-tetrol is further characterized by DTA thermogram, obtained with a temperature ramp of 10° C./min, having a prominent endotherm centered at 204° C.

40F. The crystalline androst-5-ene-3β,7β,16α,17β-tetrol of embodiment 39F wherein the 204° C. endotherm has (1) an onset temperature of 190° C. or (2) a shoulder at 217° C. or (3): (1) and (2).

41F. The crystalline androst-5-ene-3β,7β,16α,17β-tetrol of embodiment 39F or 40F wherein the DTA thermogram has a broad endotherm centered at 81° C. of variable intensity.

42F. The crystalline androst-5-ene-3β,7β,16α,17β-tetrol of embodiment 38F, 39F, 40F or 41F wherein Form Xβ 3β-tetrol is further characterized by TGA thermogram, obtained with a temperature ramp of 10° C./min, having (1) negligible % wt loss from between 60° C. to 140° C. associated with the DTA 81° C. endotherm or (2) between 5% to 10% wt loss or more associated with the prominent endotherm at about 204° C. or (3): (1) and (2).

43F. A formulation comprising or consisting essentially of one or more pharmaceutically acceptable excipients and a solid state form of androst-5-ene-3β,7β,16α,17β-tetrol provided the solid state form is not Form Iβ 3β-tetrol.

44F. The formulation of embodiment 43F wherein the formulation is an oral, parenteral, buccal, sublingual or topical formulation.

45F. The formulation of embodiment 43F or 44F wherein the solid state form is crystalline androst-5-ene-3β,7β,16α,17β-tetrol.

46F. The formulation of embodiment 43F or 45F wherein the solid state form or crystalline form is a crystalline hydrate of androst-5-ene-3β,7β,16α,17β-tetrol.

47F. The formulation of embodiment 46F wherein the crystalline hydrate is a monohydrate or a dihydrate.

48F. The formulation of embodiment 46F wherein the crystalline hydrate is Form IIβ, Form IIIβ, Form IVβ, Form Vβ or Form VIβ 3β-tetrol.

49F. The formulation of embodiment 43F or 45F wherein the solid state form or crystalline form is a crystalline anhydrate of androst-5-ene-3β,7β,16α,17β-tetrol.

50F. The formulation of embodiment 49F wherein the crystalline anhydrate is Form VIIβ, Form VIIIβ, Form IXβ or Form Xβ 3β-tetrol.

51F. A method of preparing a liquid or suspension formulation comprising admixing a solid state form of androst-5-ene-3β,7β,16α,17β-tetrol with a pharmaceutically acceptable liquid excipient provided the solid state form is not Form Iβ 3β-tetrol.

52F. The method of embodiment 26F wherein the solid state form is crystalline androst-5-ene-3β,7β,16α,17β-tetrol.

53F. The method of embodiment 51F or 52F wherein the solid state form or crystalline form is a crystalline hydrate of androst-5-ene-3β,7β,16α,17β-tetrol.

54F. The method of embodiment 53F wherein the crystalline hydrate is a monohydrate or a dihydrate.

55F. The method of embodiment 53F wherein the crystalline hydrate is Form IIβ, Form IIIβForm IVβ, Form Vβ or Form VIβ 3β-tetrol.

54F. The method of embodiment 51F or 52F wherein the solid state form or crystalline form is a crystalline anhydrate of androst-5-ene-3β,7β,16α,17β-tetrol.

55F. The method of embodiment 54F wherein the crystalline anhydrate is Form VIIβ, Form VIIIβ, Form IXβ or Form Xβ 3β-tetrol.

56F. A method of treating unwanted inflammation, comprising administering an effective amount of a solid formulation to a subject in need thereof wherein the solid formulation comprises or consists essentially of a solid state form of androst-5-ene-3β,7β,16α,17β-tetrol, provided the solid state form is not Form Iβ 3β-tetrol, and one or more pharmaceutically acceptable excipients.

57F. The method of embodiment 56F wherein the solid state form is crystalline androst-5-ene-3β,7β,16α,17β-tetrol.

58F. The method of embodiment 56F or 57F wherein the solid state form or crystalline form is a crystalline hydrate of androst-5-ene-3β,7β,16α,17β-tetrol.

60F. The method of embodiment 59F wherein the crystalline hydrate is a monohydrate or a dihydrate.

61F. The method of embodiment 58F wherein the crystalline hydrate is Form IIβ, Form IIIβForm IVβ, Form Vβ or Form VIβ 3β-tetrol.

62F. The method of embodiment 56F or 57F wherein the solid state form or crystalline form is a crystalline anhydrate of androst-5-ene-3β,7β,16α,17β-tetrol.

63F. The method of embodiment 62F wherein the crystalline anhydrate is Form VIIβ, Form VIIIβ, Form IXβ or Form Xβ 3β-tetrol.

64F. The method of any one of embodiments 56F to 63F wherein the unwanted inflammation is a condition or disease associated with chronic, non-production inflammation.

65F. The method of embodiment 64F wherein the condition or disease is an autoimmune condition or disease.

66F. The method of embodiment 64F wherein the condition or disease is a metabolic condition or disease.

67F. The method of embodiment 66F wherein the metabolic condition or disease is type 2 diabetes, obesity, insulin resistance, hyperglycemia, impaired glucose utilization or tolerance, or impaired or reduced insulin synthesis.

68F. The method of embodiment 66F wherein the metabolic condition or disease is type 2 diabetes.

69F. The method, formulation, or 3β-tetrol crystalline form of any one of embodiments 1F-67F wherein crystalline androst-5-ene-3β,7β,16α,17β-tetrol is Form IIβ 3β-tetrol.

70F. The method, formulation, or 3β-tetrol crystalline form of any one of embodiments 1F-67F wherein crystalline androst-5-ene-3β,7β,16α,17β-tetrol is Form IIIβ 3β-tetrol.

71F. The method, formulation, or 3β-tetrol crystalline form of any one of embodiments 1F-67F wherein crystalline androst-5-ene-3β,7β,16α,17β-tetrol is Form IVβ 3β-tetrol.

72F. The method, formulation, or 3β-tetrol crystalline form of any one of embodiments 1F-67F wherein crystalline androst-5-ene-3β,7β,16α,17β-tetrol is Form IVβ 3β-tetrol.

73F. The method, formulation, or 3β-tetrol crystalline form of any one of embodiments 1F-67F wherein crystalline androst-5-ene-3β,7β,16α,17β-tetrol is Form Vβ 3β-tetrol.

74F. The method, formulation, or 3β-tetrol crystalline form of any one of embodiments 1F-67F wherein crystalline androst-5-ene-3β,7β,16α,17β-tetrol is Form VIβ 3β-tetrol.

75F. The method, formulation, or 3β-tetrol crystalline form of any one of embodiments 1F-67F wherein crystalline androst-5-ene-3β,7β,16α,17β-tetrol is Form VIIβ 3β-tetrol.

76F. The method, formulation, or 3β-tetrol crystalline form of any one of embodiments 1F-67F wherein crystalline androst-5-ene-3β,7β,16α,17β-tetrol is Form VIIIβ 3β-tetrol.

77F. The method, formulation, or 3β-tetrol crystalline form of any one of embodiments 1F-67F wherein crystalline androst-5-ene-3β,7β,16α,17β-tetrol is Form IXβ 3β-tetrol.

78F. The method, formulation, or 3β-tetrol crystalline form of any one of embodiments 1F-67F wherein crystalline androst-5-ene-3β,7β,16α,17β-tetrol is Form Xβ 3β-tetrol.

For any one of embodiments 1E to 61E and 1F to 67F where XRDP and/or solid state Raman and/or DTA thermal data is recited therein, such data typically have uncertainties of ±0.2 degrees 2-theta, ±2 cm⁻¹ and ±2° C. for prominent exotherm and prominent endotherm inflection points (i.e., peak center) not associated with decomposition, respectively. Recited shoulders and onset temperatures are typically have greater uncertainties for their recited temperatures than those recited for the prominent endotherms to which they are associated. A recited shoulder may also become impedded into its associated endotherm and its presence and associated temperature uncertainty is often highly dependent on the temperature scan rate. Embodiments reciting TGA thermal data not associated with decomposition typically have ±2° C. uncertainties for each end of the recited temperature range. Such TGA temperature ranges where a weight loss is recited are typically associated with ±2 wt % uncertainties. For broad or weak endothermic or exothermic DTA, thermal transitions uncertainties double (i.e. ±2° C.) for the inflection point defining those transitions. For prominent DTA endotherms that are usually indicative of melting but are associated with significant TGA wt % losses (i.e., 5-10 wt % or more), such endotherms are typically indicative of decomposition. A transition temperature associated with such an endotherm may be referred to as compound's decomposition temperature and may be associated with an uncertainly of ±5 wt % or more. XRDP data is preferably associated with uncertainties of ±0.10 degrees 2-theta. Solid state Raman data is preferably associated with uncertainties of ±1.0 cm⁻¹, more preferably with ±0.5 cm⁻¹. Prominent DTA endotherms not associated with decomposition or prominent exotherms preferably have uncertainties of ±1° C.

1G. Use of a solid state form of androst-5-ene-3β,7β,16α,17β-tetrol in the manufacture of a medicant, provided the solid state form is not Form Iβ 3β-tetrol.

2G. The use according to embodiment 1G wherein the solid state form is crystalline androst-5-ene-3β,7β,16α,17β-tetrol.

3G. The use according to embodiment 1G wherein the solid state form is a crystalline hydrate of androst-5-ene-3β,7β,16α,17β-tetrol.

4G. The use according to embodiment 3G wherein the crystalline hydrate is a monohydrate or a dihydrate.

5G. The use according to embodiment 3G wherein the crystalline hydrate is Form IIβ, Form IIIβ, Form IVβ, Form Vβ or Form VIβ 3β-tetrol.

6G. The use according to embodiment 1G wherein the solid state form is a crystalline anhydrate of androst-5-ene-3β,7β,16α,17β-tetrol.

7G. The use according to embodiment 6G wherein the crystalline anhydrate is Form VIIβ, Form VIIIβ, Form IXβ or Form Xβ 3β-tetrol.

8G. The use according to embodiment 1G or 2G wherein the solid state or crystalline form is Form IIβ 3β-tetrol.

9G. The use according to embodiment 1G or 2G wherein the solid state or crystalline form is Form IIIβ 3β-tetrol.

10G. The use according to embodiment 1G or 2G wherein the solid state or crystalline form is Form IVβ 3β-tetrol.

11G. The use according to embodiment 1G or 2G wherein the solid state or crystalline form is Form Vβ 3βtetrol.

12G. The use according to embodiment 1G or 2G wherein the solid state or crystalline form is Form VIβ 3β-tetrol.

13G. The use according to embodiment 1G or 2G wherein the solid state or crystalline form is Form VIIβ 3β-tetrol.

14G. The use according to embodiment 1G or 2G wherein the solid state or crystalline form is Form VIIIβ 3β-tetrol.

15G. The use according to embodiment 1G or 2G wherein the solid state or crystalline form is Form IXβ 3β-tetrol.

16G. The use according to embodiment 1G or 2G wherein the solid state or crystalline form is Form Xβ 3βtetrol.

17G. Use of a solid state form of androst-5-ene-3β,7β,16α,17β-tetrol in the manufacture of a medicant for the treatment of unwanted inflammation, provided the solid state form is not Form Iβ 3β-tetrol.

18G. The use according to embodiment 18G wherein the unwanted inflammation is a condition or disease associated with chronic, non-production inflammation.

19G. The use according to embodiment 19G wherein the condition or disease is an autoimmune condition or disease.

20G. The use according to embodiment 19G wherein the condition or disease is a metabolic condition or disease.

21G. The use according to embodiment 21G wherein the metabolic condition or disease is type 2 diabetes, obesity, insulin resistance, hyperglycemia, impaired glucose utilization or tolerance, or impaired or reduced insulin synthesis.

22G. The use according to any one of embodiments 18G-22G wherein the solid state form is Form IIβ 3β-tetrol.

23G. The use according to any one of embodiments 18G-22G wherein the solid state form is Form IIIβ 3β-tetrol.

24G. The use according to any one of embodiments 18G-22G wherein the solid state form is Form IVβ 3β-tetrol.

25G. The use according to any one of embodiments 18G-22G wherein the solid state form is Form Vβ 3βtetrol.

26G. The use according to any one of embodiments 18G-22G wherein the solid state form is Form VIβ 3β-tetrol.

27G. The use according to according to any one of embodiments 18G-22G wherein the solid state form is Form VIIβ 3β-tetrol.

28G. The use according to according to any one of embodiments 18G-22G wherein the solid state form is Form VIIIβ 3β-tetrol.

29G. The use according to according to any one of embodiments 18G-22G wherein the solid state form is Form IXβ 3β-tetrol.

30G. The use according to according to any one of embodiments 18G-22G wherein the solid state form is Form Xβ 3βtetrol.

31G. A solid state form of androst-5-ene-3β,7β,16α,17β-tetrol for treating unwanted inflammation wherein the solid state form is not Form Iβ 3β-tetrol.

32G. A solid state form of androst-5-ene-3β,7β,16α,17β-tetrol for treating an autoimmune condition or disease wherein the solid state form is not Form Iβ 3β-tetrol.

33G. A solid state form of androst-5-ene-3β,7β,16α,17β-tetrol for treating a metabolic condition wherein the solid state form is not Form Iβ 3β-tetrol.

34G. A solid state from of androst-5-ene-3β,7β,16α,17β-tetrol for treating type 2 diabetes wherein the solid state form is not Form Iβ 3β-tetrol.

35G. A solid state from of androst-5-ene-3β,7β,16α,17β-tetrol for treating a lung inflammation condition or disease wherein the solid state form is not Form Iβ 3β-tetrol.

36G. A solid state form of androst-5-ene-3β,7β,16α,17β-tetrol for treating a bowel inflammation condition or disease wherein the solid state form is not Form Iβ 3β-tetrol.

37G. A solid state form of androst-5-ene-3β,7β,16α,17β-tetrol for treating a liver inflammation condition or disease wherein the solid state form is not Form Iβ 3β-tetrol.

38G. A solid state form of A solid state form of androst-5-ene-3β,7β,16α,17β-tetrol for treating an arthritis condition or disease wherein the solid state form is not Form Iβ 3β-tetrol.

39G. The solid state form of any one of embodiments 31G-38G wherein the solid state form is crystalline androst-5-ene-3β,7β,16α,17β-tetrol.

40G. The solid state form of any one of embodiments 31G-39G wherein the solid state form or crystalline from of androst-5-ene-3β,7β,16α,17β-tetrol is Form IIβ 3β-tetrol.

41G. The solid state form of any one of embodiments 31G-39G wherein the solid state form or crystalline from of androst-5-ene-3β,7β,16α,17β-tetrol is Form IIIβ 3β-tetrol.

42G. The solid state form of any one of embodiments 31G-39G wherein the solid state form or crystalline from of androst-5-ene-3β,7β,16α,17β-tetrol is Form IVβ 3β-tetrol.

43G. The solid state form of any one of embodiments 31G-39G wherein the solid state form or crystalline from of androst-5-ene-3β,7β,16α,17β-tetrol is Form Vβ 3βtetrol.

44G. The solid state form of any one of embodiments 31G-39G wherein the solid state form or crystalline from of androst-5-ene-3β,7β,16α,17β-tetrol is Form VIβ 3β-tetrol.

45G. The solid state form of any one of embodiments 31G-39G wherein the solid state form or crystalline from of androst-5-ene-3β,7β,16α,17β-tetrol is Form VIIβ 3β-tetrol.

46G. The solid state form of any one of embodiments 31G-39G wherein the solid state form or crystalline from of androst-5-ene-3β,7β,16α,17β-tetrol is Form VIIIβ 3β-tetrol.

47G. The solid state form of any one of embodiments 31G-39G wherein the solid state form or crystalline from of androst-5-ene-3β,7β,16α,17β-tetrol is Form IXβ 3β-tetrol.

48G. The solid state form of any one of embodiments 31G-39G wherein the solid state form or crystalline from of androst-5-ene-3β,7β,16α,17β-tetrol is Form Xβ 3β-tetrol.

Variations and modifications of these embodiments and other portions of this disclosure will be apparent to the skilled artisan after a reading thereof. Such variations and modifications are within the scope of this invention. The claims in this application or in applications that claim priority from this application will more particularly describe or define the invention. All citations or references cited herein are incorporated herein by reference in their entirety at this location or in additional paragraphs that follow this paragraph. Other descriptions are found in application Ser. No. 11/941,936, filed Nov. 17, 2007, U.S. provisional application Ser. No. 60/866,395, filed Nov. 17, 2006, U.S. provisional application Ser. No. 60/866,700, filed Nov. 21, 2006, U.S. provisional application Ser. No. 60/868,042, filed Nov. 30, 2006, U.S. provisional application Ser. No. 60/885,003, filed Jan. 15, 2007, U.S. provisional application Ser. No. 60/888,058, filed Feb. 2, 2007, all of which are incorporated herein by reference.

### EXAMPLES

General Methods

Raman Spectroscopy—FT-Raman spectra were acquired on a Raman accessory module interfaced to a MAGNA 860™ Fourier transform infrared (FT-IR) spectrometer (Thermo Nicolet). The module uses an excitation wavelength of 1064 nm and an indium gallium arsenide (InGaAs) detector. Approximately 1.5 W of Nd:YVO₄ laser power was used to irradiate the sample. A total of 256 sample scans were collected from 3600-100 cm⁻¹ at a spectral resolution of 4 cm⁻¹ using Happ-Genzel apodization. Wavelength calibration was preformed using sulfur and cyclohexane.

X-ray Powder Diffraction—XRPD patterns were collected using an Intel XRG-3000 diffractometer equipped with a curved position sensitive detector with a 2-theta range of 120° C. An incident beam of Cu Kα radiation (40 kV, 30 mA) was used to collect data in real time at a resolution of 0.03° 2-theta. Prior to the analysis, a silicon standard (NIST SRM 640c) was analyzed to verify the Si 111 peak position. Samples were prepared for analysis by packing them into thin-walled glass capillaries. Each capillary was mounted onto a goniometer head and rotated during data acquisition. The monochromator slit was set at 5 mm by 160 μm, and the samples were analyzed for 5 minutes.

Differential Thermal and Thermogravimetric Analyses—Thermal data was obtained on a Seiko TG/DTA 220U instrument. A 5-8 mg sample of 3β-tetrol in solid state form was loaded into an aluminum sample pan and tapped down with a glass rod. The sample, in the aluminum sample pan that was uncovered and uncrimped or covered with another pan, was equilibrated at 25° C. and heated under nitrogen purge in at a rate of 10° C./minute, unless otherwise specified, to a final temperature of 300° C.

Abbreviations used—DCM, dichloromethane; DMF, N,N′-dimethyl-formamide; TBDMSCl, t-butyl-dimethylsilyl chloride; TMSCl, trimethylsilyl chloride; ACN, acetonitrile; EtOH, ethanol, MeOH, methanol; EtOAc, ethyl acetate; Et₂O, ethyl ether; THF, tetrahydrofuran; LDA, lithium di-isopropylamide; DHEA, dehydroepiandrosterone; m-CPBA, m-chloroperbenzoic acid.

### Example 1

### Synthesis of androst-5-ene-3β,7β,16α,17β-tetrol (3β-tetrol)

The title compound was prepared according to the following reaction scheme.

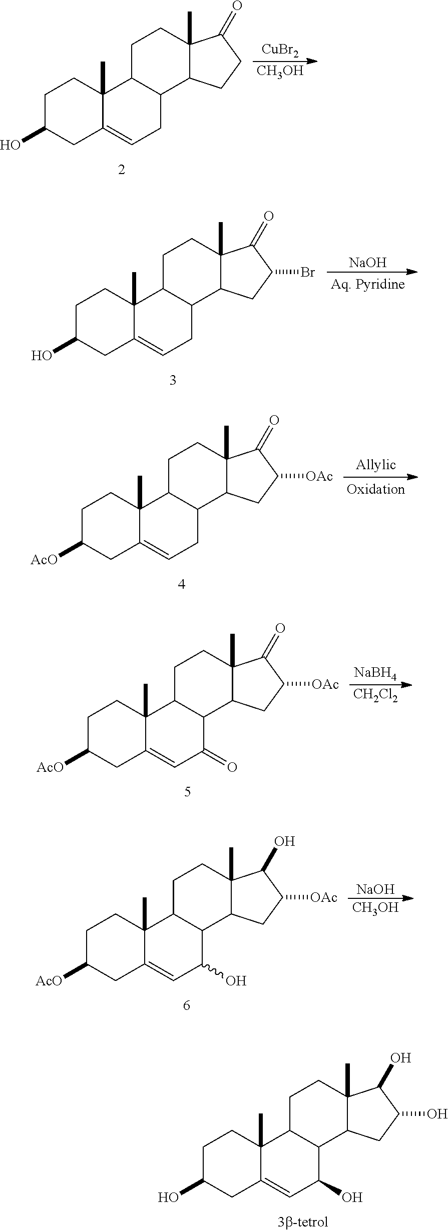

Step 1. Androst-5-en-17-one-3β,16α-diol diacetate (3). 16α-bromodehydroepiandrosterone 3 was prepared by refluxing DHEA (2) in methanol with copper (II) bromide. To 15.0 g of 2 (40.8 mmol) in pyridine (129 mL) and water (309 mL) was added 120 mL of 1N aqueous sodium hydroxide and the mixture was stirred in air for 15 minutes. The reaction mixture was poured into ice/water saturated with sodium chloride and containing excess hydrochloric acid. The crude product was filtered, washed with water until neutral and dried in vacuo over anhydrous calcium chloride at 55-60° C. Recrystallization from methanol afforded 8.21 g of 16α-hydroxy-DHEA, mp. 194.4-195.1° C. This compound was converted to the diacetate 4 by treatment with excess acetic acid in pyridine and was followed by purification with flash chromatography.

Step 2. 3β,16α-Di-acetoxy-androst-5-en-7,17-dione (5). To a solution of 4 (20.1 g, 51.7 mmol) in benzene containing celite (60 g) and pyridinium dichromate (75 g) was added 22 mL of 70% tert-butyl hydrogen peroxide. After 2 days of stirring at room temperature, diethyl ether (600 mL) was added and precipitate was filtered and washed with ether (2×100 mL). The residue was purified by flash chromatography (60% ethyl acetate in hexanes) and recrystallized to give 16.0 g (39.8 mmol, 77%) of 5 as prisms, mp. 205.6-206.2° C.

Step 3. Androst-5-ene-3β,7β,16α,17β-tetrol (3β-tetrol). To a solution of 5 (10.0 g, 24.8 mmol) in dichloromethane (75 mL) and methanol (255 mL) at 0° C. was added 1.5 g of sodium borohydride and the mixture was stirred at 0° C. for 1 hour. After quenching with acetic acid (3.5 mL) the reaction mixture was partitioned between dichloromethane and water. The organic layer was concentrated to a mixture of 7α and 7β diacetate tetrols. This mixture was purified by flash chromatography and HPLC to give 2.90 g of the 7β-diacetate epimer (9.5 mmol, 38%), mp. 216.8-220.8° C. Saponification in methanol (100 mL) with 1N sodium hydroxide (60 mL) for 2 days at room temperature provided crude 3β-tetrol.

### Example 2

### Alternative synthesis of androst-5-ene-3β,7β,16α,17β-tetrol (3β-tetrol)

The title compound was alternatively prepared according to the following reaction scheme.

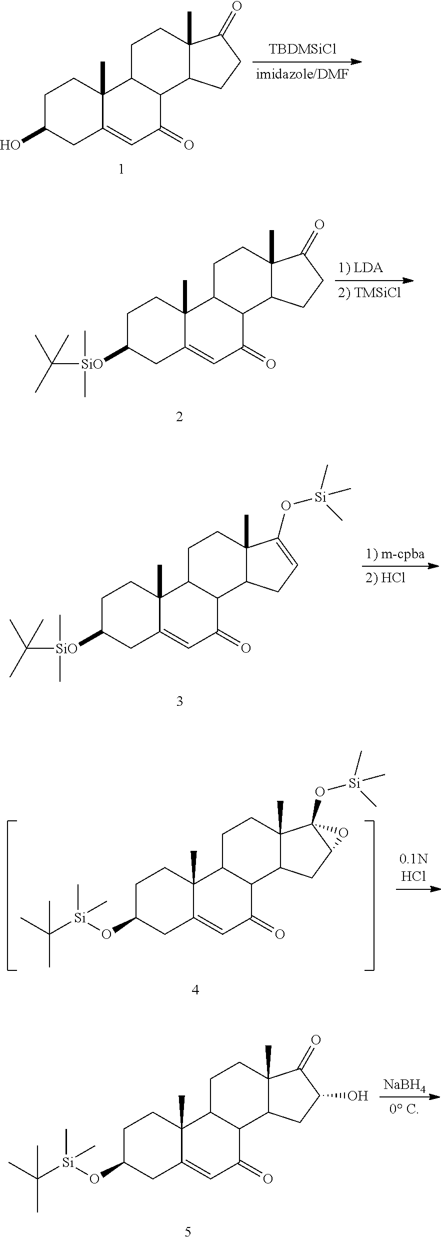

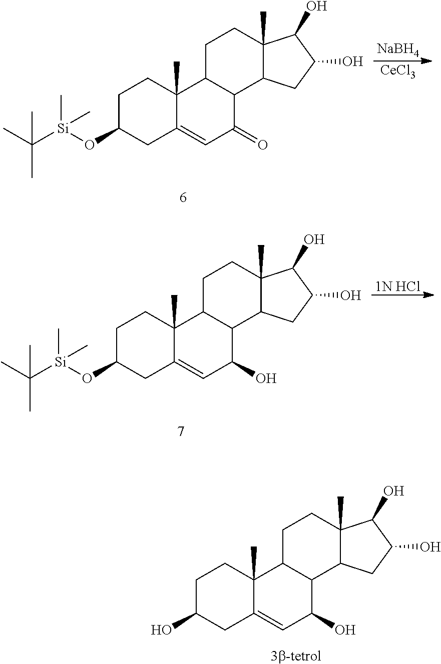

Step 1. 3β-(t-Butyl-dimethylsilyl)-oxy-androst-5-en-7,17-dione (2): To a solution of 20 g (66 mmol) androst-5-en-7,17-dione-3β-ol and 5.5 g (80 mmol) imidazole in 250 mL DMF was added 12.3 g (80 mmol) TBDMSCl. After stirring overnight at room temperature, 300 mL water was added to give a slurry whose solids were collected by vacuum filtration, washed with water and dried in vacuo to provide 27.1 g 2 (99% yield).

Step 2. 17-(Trimethylsilyl)oxy-3β-(t-Butyl-dimethylsilyl)-oxy-androst-5,16-dien-7-one (3): To a solution of 2 (10 g, 24 mmol) in anhydrous THF (250 mL) cooled to −78° C. was added LDA (2 M in THF/heptane/ethylbenzene, 18 mL, 36 mmol) over 5 min. After stirring for 30 min, TMS—Cl (4.8 mL, 36 mmol) was then added dropwise to the reaction mixture over 10 min. After stirring for 30 min, a second batch of LDA (2 M in THF/heptane/ethylbenzene, 18 mL, 36 mmol) was added to the reaction mixture over 5 min, which was then stirred for 30 min. A second batch of TMS—Cl (4.8 mL, 36 mmol) was then added dropwise over 10 min. After stirring for 30 min, the reaction mixture was allowed to warm up to room temperature and stirred for 1 hr. After quenching with water (250 mL), the reaction mixture was extracted with ether (100 mL×2), and the combined extracts were washed with water and brine and dried over Na₂SO₄ to yield 11.7 g 3 (100% yield).

Step 3. 3β-(t-Butyl-dimethylsilyl)-oxy-androst-5-en-7,17-dione-16α-ol (5): To a solution of 3 (10.5 g, 21.5 mmol) in THF (300 mL), cooled to 0° C. was added m-CPBA (77% purity, 5.3 g, 23.5 mmol) in aliquots over 10 min. The solution was warmed to room temperature and stirred for 30 min to provide 4 as an intermediate in solution. The solution was then treated with 50 mL of 0.1 N HCl and stirred for 10 min. The pH of the solution was adjusted to 7 with saturated NaHCO₃ and extracted with EtOAc (100 mL×3). The combined organic extracts were washed with water, saturated NaHCO₃ and brine, and dried over Na₂SO₄. EtOAc was removed in vacuo to yield the crude product (˜10 g). The crude was loaded onto 25 g of silica gel using methylene chloride and purified on a 100 g silica gel column by elution with 100% Hex (250 mL) to remove excess m-CPBA and other non-polar impurities followed by 50% Hexanes/EtOAc (500 mL). The solvent was then removed to afford 5 in 90% purity (7.5 g, 81% yield).

Step 4. 3β-(t-Butyl-dimethylsilyl)-oxy-androst-5-ene-7β,16α,17β-triol (7): To a solution of 5 (7.5 g, 17.3 mmol) in a mixed solvent of THF (150 ml) and MeOH (150 mL) cooled to 0° C. was added NaBH₄ (990 mg, 26.2 mmol) to form 6 in situ. After 10 min, a solution of CeCl₃ (7.5 g, 20 mmol) in MeOH (50 ml) pre-cooled to below −20° C. was added all at once. Additional NaBH₄ (990 mg, 26.2 mmol) was subsequently added. The solution was stirred for 30 min and quenched with 3.5 mL acetic acid. Water (800 ml) was added to form a precipitate. The solids were collected by filtration, washed with water and dried in vacuo to yield crude product 7 (7.0 g, 92% yield).

Step 6. Androst-5-ene-3β,7β,16α,17β-tetrol (3β-tetrol): To a solution of crude 7 (7 g, 16 mmol) in MeOH (120) was added HCl (1 N, 20 mL). After stirring for 10 min at room temperature, the pH of the solution was adjusted to 7 with saturated NaHCO₃. then concentrated in vacuo. As the solvent was being removed, an oil was formed which pooled in the flask. The clear solution was decanted and water was added to precipitate a solid that was collected by vacuum filtration and washed with water. The resulting crude product was recrystallized three times from MeOH to yield 3β-tetrol (2.4 g, 47%).

### Example 3

### Preparation of Crystalline Form Iβ 3β-tetrol

The crude 3β-tetrol from Example 1 was purified by HPLC using an acetonitrile-water gradient. Concentration in vacuo of the pooled fractions that contained purified 3β-tetrol resulted in fine needles (1.41 g, 4.4 mmol, 46%), mp. 202.1-206.4° C.; [α]D+1.35 (methanol, c=1). Selected ¹H NMR peaks (CD₃OD): δ 0.77 (s, 3H), 1.01 (s, 3H), 3.39 (d, 1H), 3.46 (m, 1H), 3.74 (t, 1H), 4.04 (m, 1H), 5.55 (dd, 1H).

TABLE 2
Observed XRPD peaks for Form Iβ 3β-tetrol

| °2θ | d space (Å) | Intensity (%) |
|---|---|---|
| 7.4 ± 0.1 | 11.930 ± 0.163 | 10 |
| 10.2 ± 0.1 | 8.698 ± 0.086 | 1 |
| 13.1 ± 0.1 | 6.753 ± 0.052 | 4 |
| 14.5 ± 0.1 | 6.101 ± 0.042 | 9 |
| 14.8 ± 0.1 | 5.978 ± 0.040 | 27 |
| 15.9 ± 0.1 | 5.574 ± 0.035 | 100 |
| 17.3 ± 0.1 | 5.123 ± 0.030 | 28 |
| 17.8 ± 0.1 | 4.969 ± 0.028 | 8 |
| 19.2 ± 0.1 | 4.616 ± 0.024 | 17 |
| 19.8 ± 0.1 | 4.477 ± 0.022 | 7 |
| 20.2 ± 0.1 | 4.405 ± 0.022 | 12 |
| 20.4 ± 0.1 | 4.360 ± 0.021 | 16 |
| 22.2 ± 0.1 | 4.004 ± 0.018 | 2 |
| 22.7 ± 0.1 | 3.911 ± 0.017 | 6 |
| 23.2 ± 0.1 | 3.826 ± 0.016 | 2 |
| 23.8 ± 0.1 | 3.740 ± 0.016 | 3 |
| 24.4 ± 0.1 | 3.654 ± 0.015 | 11 |
| 24.8 ± 0.1 | 3.584 ± 0.014 | 5 |
| 25.6 ± 0.1 | 3.485 ± 0.013 | 8 |
| 26.4 ± 0.1 | 3.380 ± 0.013 | 1 |
| 27.4 ± 0.1 | 3.260 ± 0.012 | 12 |
| 27.7 ± 0.1 | 3.222 ± 0.011 | 3 |
| 28.3 ± 0.1 | 3.155 ± 0.011 | 4 |
| 29.0 ± 0.1 | 3.075 ± 0.010 | 4 |
| 29.4 ± 0.1 | 3.038 ± 0.010 | 15 |

TABLE 3
Peak listing for absorptions for Raman spectrum of Form Iβ

| cm⁻¹ | Intensity |
|---|---|
| 183 | 1.31 |
| 216 | 2.69 |
| 235 | 1.76 |
| 241 | 1.74 |
| 287 | 1.38 |
| 301 | 1.30 |
| 345 | 2.45 |
| 386 | 2.41 |
| 445 | 3.24 |
| 472 | 2.17 |
| 492 | 0.83 |
| 517 | 1.60 |
| 536 | 1.70 |
| 561 | 0.91 |
| 573 | 1.13 |
| 598 | 2.06 |
| 615 | 0.56 |
| 634 | 1.20 |
| 661 | 2.73 |
| 700 | 2.36 |
| 735 | 0.52 |
| 779 | 2.89 |
| 810 | 1.06 |
| 848 | 1.62 |
| 864 | 1.51 |
| 881 | 1.32 |
| 901 | 1.88 |
| 926 | 1.23 |
| 956 | 1.25 |
| 987 | 1.89 |
| 1009 | 2.07 |
| 1024 | 1.45 |
| 1057 | 2.38 |
| 1091 | 1.78 |
| 1099 | 1.74 |
| 1126 | 2.54 |
| 1147 | 1.68 |
| 1174 | 2.10 |
| 1194 | 2.16 |
| 1217 | 1.42 |
| 1223 | 1.43 |
| 1234 | 1.57 |
| 1246 | 1.52 |
| 1277 | 2.03 |
| 1327 | 2.32 |
| 1344 | 2.55 |
| 1373 | 1.55 |
| 1437 | 4.54 |
| 1460 | 2.42 |
| 1670 | 4.25 |
| 2663 | 0.52 |
| 2740 | 0.52 |
| 2835 | 3.94 |
| 2856 | 6.93 |
| 2870 | 6.76 |
| 2893 | 7.44 |
| 2902 | 7.72 |
| 2937 | 11.07 |
| 2968 | 7.05 |
| 3030 | 0.83 |
| 3342 | 0.50 |
| 3352 | 0.51 |
| 3365 | 0.51 |
| 3375 | 0.53 |
| 3384 | 0.54 |
| 3398 | 0.54 |
| 3402 | 0.54 |
| 3417 | 0.50 |

The DTA thermogram of Form Iβ, obtained with the sample uncovered, exhibits a broad endotherm centered between about 98° C. to about 102° C. that is associated with a weight loss of about 5% in the thermogravimetric thermogram from between about 60° C. to about 140° C., with a temperature scan rate of 10° C./min and is consistent with Form Iβ existing as a monohydrate. The DTA thermogram additionally exhibits a prominent endotherm centered at about 204° C. (onset at about 194° C.) followed by a weaker endotherm centered at about 224° C. that is associated with additional weight loss in the thermogravimetric thermogram. These temperature transitions are consistent with partial transformation of a dehydrated pseudopolymorph to a more stable polymorphic form that subsequently melts or melting of the dehydrated pseudopolymorph which decomposes with further heating.

Form Iβ, prepared according to the forgoing method, produces crystalline material with the morphology of needles. Due to the high surface area to volume ratio of this type of crystalline material for 3β-tetrol is expected to have a favorable dissolution rate in various liquid excipients such as water and ethanol. However, needles may have unfavorable mechanical properties such as processability and flowability with respect to bulk production. Other crystalline forms of 3β-tetrol disclosed herein are more granular in morphology and thus are expected to overcome these unfavorable properties.

### Example 4

### Preparation of Crystalline Form IIβ 3β-tetrol

50 mg of crystalline Form Vβ was dissolved in 0.5 mL of reagent grade hot ethanol (90%) then 0.5 mL of acetonitrile was added to the solution. The sample vial was capped and the mixture was allowed to stand for 4 days at room temperature. The resulting crystals were collected by vacuum filtration, washed with acetonitrile and dried to provide 30 mg of Form IIβ.

The DTA thermogram of Form IIβ, obtained with the sample uncovered, exhibits a broad endotherm centered at about 102° C. that is associated with a weight loss of about 5% in the thermogravimetric thermogram from between about 60° C. to about 140° C., with a temperature scan rate of 10° C./min and is consistent with Form IIβ existing as a monohydrate. The DTA thermogram additionally exhibits a endotherm centered at about 239° C. (onset at about 235° C.) followed by an exotherm at about 242° C. and a prominent endotherm centered at about 252° C. These temperature transitions are consistent with transformation of a dehydrated pseudopolymorph to a more stable polymorphic form which then undergoes a final melt.

This crystalline hydrate form of 3β-tetrol is expected to have greater stability with respect to anhydrate crystalline forms of 3β-tetrol such as Form VIIβ, Form VIIIβ, Form IXβ and Form Xβ due to the expected greater hygroscopicity of these anhydrate forms. However, a crystalline hydrate, such as Form IIβ, Form IIIβ, Form Vβ or Form VIβ, typically will have lower dissolution rates in water or aqueous excipients as compared to these anhydrate crystalline forms, which may unfavorably impact oral bioavailability or preparation of aqueous-based formulations. However, the anhydrate crystalline forms are expected to have the higher dissolution rates in other liquid excipients in which water is miscible such as ethanol. Thus, preference for a crystalline hydrate or anhydrate of 3β-tetrol will be context dependent, i.e. dependent on route of administration or formulation process or excipient identity. Form IIβ, in addition to the therapeutic uses disclosed herein, is also useful as a precursor in preparation of other crystalline form of 3β-tetrol, e.g., Form VIIβ.

### Example 5

### Crystalline Form IIIβ 3βtetrol

500 mg of 3β-tetrol obtained during the preparation of Form VIIIβ (immediately prior to final drying of collected solids from EtOAc precipitation) was dissolved in a mixture of 5 mL of denatured ethanol and 5 mL of acetonitrile with heating in a 70° C. water bath. While gradually cooling the EtOH-ACN solution to room temperature, a seed crystal of Form VIIβ 3β-tetrol was added. The resulting crystals, formed after standing overnight at room temperature, were collected by vacuum filtration and dried in vacuo to provide 150 mg of Form IIIβ.

TABLE 4
Observed XRPD peaks for Form IIIβ 3β-tetrol

| °2θ | d space (Å) | Intensity (%) |
|---|---|---|
| 7.6 ± 0.1 | 11.602 ± 0.154 | 7 |
| 14.8 ± 0.1 | 5.978 ± 0.040 | 7 |
| 15.4 ± 0.1 | 5.769 ± 0.038 | 8 |
| 16.1 ± 0.1 | 5.502 ± 0.034 | 100 |
| 17.3 ± 0.1 | 5.114 ± 0.029 | 5 |
| 18.0 ± 0.1 | 4.928 ± 0.027 | 3 |
| 18.2 ± 0.1 | 4.880 ± 0.027 | 2 |
| 19.9 ± 0.1 | 4.471 ± 0.022 | 7 |
| 25.4 ± 0.1 | 3.514 ± 0.014 | 9 |
| 27.4 ± 0.1 | 3.260 ± 0.012 | 2 |
| 28.5 ± 0.1 | 3.135 ± 0.011 | 3 |
| 29.6 ± 0.1 | 3.020 ± 0.010 | 6 |

TABLE 5
Peak listing for absorptions for Raman spectrum of Form IIIβ

| cm⁻¹ | Intensity |
|---|---|
| 183 | 0.92 |
| 216 | 1.87 |
| 239 | 1.28 |
| 262 | 0.62 |
| 285 | 0.92 |
| 303 | 0.83 |
| 345 | 1.67 |
| 386 | 1.52 |
| 445 | 2.04 |
| 459 | 1.19 |
| 474 | 1.44 |
| 492 | 0.57 |
| 517 | 1.17 |
| 534 | 1.01 |
| 561 | 0.59 |
| 573 | 0.75 |
| 598 | 1.37 |
| 615 | 0.36 |
| 634 | 0.83 |
| 661 | 1.94 |
| 700 | 1.72 |
| 735 | 0.33 |
| 779 | 2.14 |
| 810 | 0.67 |
| 839 | 0.97 |
| 848 | 1.12 |
| 864 | 0.97 |
| 879 | 0.84 |
| 901 | 1.23 |
| 926 | 0.75 |
| 958 | 0.79 |
| 987 | 1.37 |
| 1009 | 1.25 |
| 1024 | 0.90 |
| 1057 | 1.47 |
| 1091 | 1.25 |
| 1099 | 1.13 |
| 1128 | 1.56 |
| 1147 | 1.07 |
| 1174 | 1.25 |
| 1194 | 1.49 |
| 1232 | 1.10 |
| 1246 | 0.96 |
| 1275 | 1.30 |
| 1329 | 1.52 |
| 1344 | 1.74 |
| 1371 | 1.04 |
| 1381 | 0.94 |
| 1437 | 2.83 |
| 1460 | 1.62 |
| 1670 | 2.95 |
| 2663 | 0.39 |
| 2740 | 0.39 |
| 2787 | 0.41 |
| 2858 | 4.74 |
| 2870 | 4.76 |
| 2891 | 5.29 |
| 2937 | 7.46 |
| 2964 | 4.94 |
| 3030 | 0.56 |
| 3311 | 0.34 |
| 3329 | 0.34 |
| 3354 | 0.34 |
| 3375 | 0.34 |
| 3383 | 0.34 |

The DTA thermogram of Form IIIβ, obtained with the sample covered, exhibits a broad endotherm centered at about 103° C. that is associated with a weight loss of about 5% in the thermogravimetric thermogram from between about 60° C. to about 140° C., with a temperature scan rate of 10° C./min and is consistent with Form IIIβ existing as a monohydrate. The DTA thermogram additionally exhibits a prominent endotherm centered at about 200° C. with a shoulder at about 210° C. These later temperature transitions are accompanied by weight loss in the thermogravimetric thermogram indicating that decomposition is occurring.

### Example 6

### Crystalline Form IVβ 3β-tetrol

300 mg of crystalline Form VIIIβ was dissolved in a mixture of 3 mL of denatured ethanol and 3 mL of heptanes with heating in a 70° C. water bath. The sample was set aside to cool and crystallize overnight. The resulting crystals were collected by vacuum filtration and dried in vacuo to provide 280 mg of Form IVβ.

TABLE 6
Observed XRPD peaks for Form IVβ 3β-tetrol

| °2θ | d space (Å) | Intensity (%) |
|---|---|---|
| 7.7 ± 0.1 | 11.512 ± 0.152 | 10 |
| 10.4 ± 0.1 | 8.547 ± 0.083 | 1 |
| 12.2 ± 0.1 | 7.231 ± 0.059 | 1 |
| 13.5 ± 0.1 | 6.545 ± 0.049 | 1 |
| 14.8 ± 0.1 | 6.002 ± 0.041 | 4 |
| 15.4 ± 0.1 | 5.769 ± 0.038 | 15 |
| 16.2 ± 0.1 | 5.482 ± 0.034 | 100 |
| 18.3 ± 0.1 | 4.856 ± 0.026 | 3 |
| 19.6 ± 0.1 | 4.518 ± 0.023 | 8 |
| 19.9 ± 0.1 | 4.464 ± 0.022 | 6 |
| 20.8 ± 0.1 | 4.273 ± 0.020 | 5 |
| 22.3 ± 0.1 | 3.994 ± 0.018 | 2 |
| 23.1 ± 0.1 | 3.845 ± 0.016 | 2 |
| 24.0 ± 0.1 | 3.708 ± 0.015 | 1 |
| 25.3 ± 0.1 | 3.518 ± 0.014 | 12 |
| 26.6 ± 0.1 | 3.350 ± 0.012 | 1 |
| 28.2 ± 0.1 | 3.168 ± 0.011 | 5 |
| 28.6 ± 0.1 | 3.119 ± 0.011 | 3 |
| 29.8 ± 0.1 | 2.996 ± 0.010 | 5 |

TABLE 7
Peak listing for absorptions for Raman spectrum of Form IVβ

| cm⁻¹ | Intensity |
|---|---|
| 185 | 1.60 |
| 214 | 3.70 |
| 235 | 2.36 |
| 285 | 1.82 |
| 301 | 1.66 |
| 345 | 3.00 |
| 386 | 2.54 |
| 443 | 3.88 |
| 474 | 3.00 |
| 517 | 2.02 |
| 536 | 2.06 |
| 559 | 1.10 |
| 575 | 1.36 |
| 598 | 2.44 |
| 634 | 1.43 |
| 661 | 3.36 |
| 700 | 2.77 |
| 735 | 0.58 |
| 779 | 3.39 |
| 808 | 1.23 |
| 848 | 1.91 |
| 864 | 1.62 |
| 879 | 1.58 |
| 901 | 2.03 |
| 926 | 1.40 |
| 958 | 1.36 |
| 985 | 2.14 |
| 1009 | 2.16 |
| 1024 | 1.49 |
| 1045 | 2.16 |
| 1055 | 2.26 |
| 1090 | 2.00 |
| 1099 | 1.96 |
| 1126 | 2.60 |
| 1147 | 1.72 |
| 1172 | 2.19 |
| 1194 | 2.39 |
| 1226 | 1.84 |
| 1246 | 1.66 |
| 1279 | 2.10 |
| 1304 | 1.85 |
| 1329 | 2.69 |
| 1342 | 2.98 |
| 1375 | 1.70 |
| 1437 | 5.07 |
| 1460 | 2.76 |
| 1558 | 0.48 |
| 1641 | 0.60 |
| 1670 | 5.08 |
| 2661 | 0.57 |
| 2669 | 0.56 |
| 2708 | 0.49 |
| 2713 | 0.51 |
| 2719 | 0.53 |
| 2727 | 0.53 |
| 2742 | 0.57 |
| 2748 | 0.56 |
| 2758 | 0.55 |
| 2866 | 7.80 |
| 2891 | 8.55 |
| 2900 | 8.46 |
| 2937 | 12.43 |
| 2966 | 7.81 |
| 3022 | 1.01 |
| 3032 | 1.02 |
| 3269 | 0.51 |
| 3278 | 0.51 |
| 3286 | 0.53 |
| 3290 | 0.53 |
| 3302 | 0.56 |
| 3321 | 0.62 |
| 3327 | 0.61 |
| 3332 | 0.61 |
| 3338 | 0.62 |
| 3344 | 0.61 |
| 3352 | 0.64 |
| 3365 | 0.62 |
| 3373 | 0.64 |
| 3383 | 0.64 |
| 3396 | 0.61 |
| 3404 | 0.59 |
| 3411 | 0.58 |
| 3421 | 0.55 |

The DTA thermogram of Form IVβ, obtained with the sample covered, exhibits a broad endotherm centered at about 99° C. that is associated with a weight loss of about 5% in the thermogravimetric thermogram from between about 60° C. to about 140° C., with a temperature scan rate of 10° C./min and is consistent with Form IVβ existing as a monohydrate. The DTA thermogram additionally exhibits a weak endotherm centered at about 197° C. (onset at about 189° C.) and a more prominent endotherm centered at about 233° C. (onset at about 227° C.). These later temperature transitions are consistent with partial melting of a dehydrated pseudopolymorph with subsequent reorganization to a more stable polymorphic form (whose associated exothermic is not observed) that finally melts.

### Example 7

### Crystalline Form Vβ 3βtetrol

1.9 grams of 3β-tetrol from Example 2 was dissolved in methanol resulting in a yellow insoluble solid that was removed by filtration through 11 micron filter paper. The methanol solution was then allowed to cool to room temperature and the resulting crystals were collected and dried over P₂O₅ in vacuo (<2 torr) at 80° C. overnight to provide 1.5 g of Form V.

TABLE 8
Observed XRPD peaks for Form Vβ 3β-tetrol

| °2θ | d space (Å) | Intensity (%) |
|---|---|---|
| 7.4 ± 0.1 | 11.930 ± 0.163 | 7 |
| 13.1 ± 0.1 | 6.753 ± 0.052 | 3 |
| 14.6 ± 0.1 | 6.076 ± 0.042 | 10 |
| 14.8 ± 0.1 | 5.978 ± 0.040 | 19 |
| 15.9 ± 0.1 | 5.564 ± 0.035 | 100 |
| 17.3 ± 0.1 | 5.123 ± 0.030 | 21 |
| 17.8 ± 0.1 | 4.978 ± 0.028 | 5 |
| 19.2 ± 0.1 | 4.616 ± 0.024 | 11 |
| 19.6 ± 0.1 | 4.525 ± 0.023 | 4 |
| 19.8 ± 0.1 | 4.477 ± 0.022 | 6 |
| 20.1 ± 0.1 | 4.411 ± 0.022 | 8 |
| 20.3 ± 0.1 | 4.366 ± 0.021 | 10 |
| 21.1 ± 0.1 | 4.207 ± 0.020 | 3 |
| 22.7 ± 0.1 | 3.911 ± 0.017 | 4 |
| 23.2 ± 0.1 | 3.826 ± 0.016 | 2 |
| 23.7 ± 0.1 | 3.750 ± 0.016 | 3 |
| 24.4 ± 0.1 | 3.654 ± 0.015 | 7 |
| 24.8 ± 0.1 | 3.584 ± 0.014 | 4 |
| 25.6 ± 0.1 | 3.485 ± 0.013 | 6 |
| 27.3 ± 0.1 | 3.267 ± 0.012 | 7 |
| 27.7 ± 0.1 | 3.222 ± 0.011 | 3 |
| 28.3 ± 0.1 | 3.155 ± 0.011 | 4 |
| 29.4 ± 0.1 | 3.035 ± 0.010 | 13 |

TABLE 9
Peak listing for absorptions for Raman spectrum of Form Vβ

| cm⁻¹ | Intensity |
|---|---|
| 187 | 3.54 |
| 214 | 7.34 |
| 285 | 3.94 |
| 297 | 3.72 |
| 345 | 5.59 |
| 374 | 3.79 |
| 386 | 4.61 |
| 443 | 6.98 |
| 472 | 5.93 |
| 517 | 3.51 |
| 536 | 3.83 |
| 561 | 2.32 |
| 575 | 2.73 |
| 598 | 4.13 |
| 636 | 2.96 |
| 661 | 5.55 |
| 700 | 4.23 |
| 735 | 1.37 |
| 779 | 5.26 |
| 808 | 2.32 |
| 848 | 3.40 |
| 862 | 3.39 |
| 879 | 3.12 |
| 901 | 3.47 |
| 926 | 2.65 |
| 958 | 2.56 |
| 985 | 3.68 |
| 1009 | 4.09 |
| 1055 | 4.15 |
| 1076 | 2.54 |
| 1090 | 3.48 |
| 1099 | 3.52 |
| 1126 | 4.91 |
| 1147 | 3.26 |
| 1172 | 4.12 |
| 1194 | 4.25 |
| 1228 | 3.32 |
| 1250 | 3.41 |
| 1279 | 3.85 |
| 1329 | 5.06 |
| 1344 | 5.53 |
| 1379 | 3.37 |
| 1439 | 10.13 |
| 1460 | 5.66 |
| 1597 | 1.39 |
| 1637 | 3.13 |
| 1670 | 8.86 |
| 2868 | 13.10 |
| 2893 | 14.77 |
| 2902 | 14.77 |
| 2937 | 21.54 |
| 2966 | 13.47 |
| 3028 | 2.09 |

The DTA thermogram of Form Vβ, obtained with the sample uncovered, exhibits a broad endotherm centered between about 98° C. to about 102° C. that is associated with a weight loss of about 5% in the thermogravimetric thermogram from between about 60° C. to about 140° C., with a temperature scan rate of 10° C./min and is consistent with Form Vβ existing as a monohydrate. The DTA thermogram additionally exhibits a weak exotherm centered between about 188-190° C. and a prominent endotherm centered at about 230° C. (onset at about 223° C.) These temperature transitions are consistent with of a dehydrated pseudopolymorph to a more stable polymorphic form which then finally melts. The XRPD of form Vβ is substantially identical to the XRPD of Form Iβ. However, these two forms exhibit significantly different DTA thermographic traces, thus Form Iβ and Form Vβ are unique crystalline forms that are isostructural.

As a monohydrate, crystalline Form Vβ is expected to have greater stability with exposure to atmospheric moisture due to its lower hygroscopicity as compared to the anhydrate forms. Form Vβ is also expected to have a better dissolution rate in water miscible excipients such as ethanol, but a poorer dissolution rate in water, which may unfavorably impact oral bioavailability. Thus, preference for Form Vβ over another crystalline hydrate or an anhydrate of 3β-tetrol will be context dependent, i.e. dependent on route of administration or formulation process or excipient identity. Form Vβ, in addition to the therapeutic uses disclosed herein, is also useful as a precursor in preparation of other crystalline form of 3β-tetrol, e.g., Form IIβ.

### Example 8

### Crystalline Form VIβ 3β-tetrol

2.5 grams of Form IXβ 3β-tetrol was dissolved in methanol and passed through a 0.45 micron filter, then allowed to concentrate in vacuo (20-50 torr). The resulting crystalline material was collected by vacuum filtration and dried in vacuo (<2 torr) over P₂O₅ at 80° C. overnight to provide 2.3 grams of Form VIβ.

TABLE 10
Observed XRPD peaks for Form VIβ 3β-tetrol

| °2θ | d space (Å) | Intensity (%) |
|---|---|---|
| 6.5 ± 0.1 | 13.516 ± 0.210 | 9 |
| 7.7 ± 0.1 | 11.543 ± 0.152 | 10 |
| 7.9 ± 0.1 | 11.177 ± 0.143 | 4 |
| 8.2 ± 0.1 | 10.834 ± 0.134 | 8 |
| 9.7 ± 0.1 | 9.104 ± 0.094 | 5 |
| 10.4 ± 0.1 | 8.533 ± 0.083 | 3 |
| 10.9 ± 0.1 | 8.128 ± 0.075 | 2 |
| 12.2 ± 0.1 | 7.272 ± 0.060 | 5 |
| 13.1 ± 0.1 | 6.763 ± 0.052 | 60 |
| 14.3 ± 0.1 | 6.197 ± 0.043 | 4 |
| 15.0 ± 0.1 | 5.915 ± 0.040 | 69 |
| 15.4 ± 0.1 | 5.768 ± 0.038 | 20 |
| 15.8 ± 0.1 | 5.592 ± 0.035 | 9 |
| 16.2 ± 0.1 | 5.477 ± 0.034 | 100 |
| 16.7 ± 0.1 | 5.298 ± 0.032 | 9 |
| 17.0 ± 0.1 | 5.231 ± 0.031 | 56 |
| 17.9 ± 0.1 | 4.950 ± 0.028 | 3 |
| 18.3 ± 0.1 | 4.838 ± 0.026 | 3 |
| 19.3 ± 0.1 | 4.590 ± 0.024 | 10 |
| 19.6 ± 0.1 | 4.524 ± 0.023 | 10 |
| 19.9 ± 0.1 | 4.452 ± 0.022 | 15 |
| 20.8 ± 0.1 | 4.262 ± 0.020 | 7 |
| 21.1 ± 0.1 | 4.212 ± 0.020 | 8 |
| 21.2 ± 0.1 | 4.185 ± 0.020 | 10 |
| 21.9 ± 0.1 | 4.065 ± 0.018 | 6 |
| 22.3 ± 0.1 | 3.987 ± 0.018 | 14 |
| 22.8 ± 0.1 | 3.900 ± 0.017 | 5 |
| 23.1 ± 0.1 | 3.853 ± 0.017 | 9 |
| 23.5 ± 0.1 | 3.785 ± 0.016 | 7 |
| 23.9 ± 0.1 | 3.718 ± 0.015 | 12 |
| 24.6 ± 0.1 | 3.618 ± 0.015 | 6 |
| 25.0 ± 0.1 | 3.556 ± 0.014 | 5 |
| 25.4 ± 0.1 | 3.513 ± 0.014 | 12 |
| 25.9 ± 0.1 | 3.441 ± 0.013 | 4 |
| 26.4 ± 0.1 | 3.381 ± 0.013 | 3 |
| 27.1 ± 0.1 | 3.295 ± 0.012 | 3 |
| 28.2 ± 0.1 | 3.165 ± 0.011 | 5 |
| 28.7 ± 0.1 | 3.111 ± 0.011 | 4 |
| 29.8 ± 0.1 | 2.996 ± 0.010 | 7 |

TABLE 11
Peak listing for absorptions for Raman spectrum of Form VIβ

| cm⁻¹ | Intensity |
|---|---|
| 181 | 1.51 |
| 216 | 4.07 |
| 243 | 2.53 |
| 283 | 1.74 |
| 307 | 1.32 |
| 341 | 2.88 |
| 372 | 1.43 |
| 384 | 2.26 |
| 440 | 3.24 |
| 447 | 3.52 |
| 476 | 2.88 |
| 519 | 2.31 |
| 532 | 1.62 |
| 561 | 0.86 |
| 577 | 1.26 |
| 598 | 2.73 |
| 634 | 1.58 |
| 661 | 3.16 |
| 696 | 3.01 |
| 735 | 0.75 |
| 779 | 3.88 |
| 810 | 1.22 |
| 839 | 1.59 |
| 847 | 2.29 |
| 862 | 1.97 |
| 879 | 1.68 |
| 901 | 2.54 |
| 926 | 1.36 |
| 951 | 1.2 |
| 958 | 1.46 |
| 966 | 1.18 |
| 987 | 2.49 |
| 1007 | 2.24 |
| 1022 | 1.72 |
| 1036 | 2.19 |
| 1059 | 2.59 |
| 1091 | 3.14 |
| 1126 | 3.17 |
| 1149 | 2.19 |
| 1174 | 2.31 |
| 1196 | 2.56 |
| 1215 | 1.2 |
| 1232 | 2.35 |
| 1250 | 2.02 |
| 1257 | 1.83 |
| 1273 | 2.57 |
| 1319 | 2.97 |
| 1344 | 3.73 |
| 1375 | 1.89 |
| 1439 | 6.84 |
| 1462 | 3.49 |
| 1670 | 5.16 |
| 2854 | 7.72 |
| 2871 | 7.68 |
| 2891 | 8.91 |
| 2937 | 13.95 |
| 2954 | 9.4 |
| 2970 | 8.69 |
| 3030 | 0.97 |
| 3057 | 0.84 |
| 3269 | 0.75 |
| 3278 | 0.78 |
| 3286 | 0.8 |
| 3294 | 0.8 |
| 3305 | 0.81 |
| 3311 | 0.82 |
| 3323 | 0.8 |
| 3330 | 0.76 |

The DTA thermogram of Form VIβ, obtained with the sample covered, exhibits a prominent endotherm centered at about 111° C. (onset at about 107° C.) that is associated with a weight loss of about 10% in the thermogravimetric thermogram from between about 60° C. to about 140° C., with a temperature scan rate of 10° C./min and is consistent with Form VIβ existing as a di-hydrate The DTA thermogram additionally exhibits a prominent endotherm at about 233° C. (onset at about 226° C.).

This crystalline hydrate form of 3β-tetrol is expected to have greater stability with respect to the monohydrate crystalline forms, e.g., Form IIβ, Form IIIβ, Form IVβ and Form Vβ, and anhydrate crystalline forms of 3β-tetrol, e.g., Form VIIβ, Form VIIIβ, Form IXβ and

Form Xβ due to the expected greater hygroscopicity of these monohydrate and anhydrate forms. However, this crystalline di-hydrate is expected to have the lowest dissolution rates in water or aqueous excipients, which may unfavorably impact oral bioavailability or preparation of aqueous-based formulations, but is expected to have the highest dissolution rate in water miscible solvents such as ethanol, which is a pharmaceutically acceptable liquid excipient. The anhydrate crystalline forms are expected to have the lowest dissolution rates in water or aqueous excipients with the mono-hydrates having dissolution rates intermediate to Form VIβ and the anhydrate. Thus, preference Form VIβ 3β-tetrol over a crystalline monohydrate or anhydrate of 3β-tetrol will be context dependent, i.e. dependent on route of administration or formulation process.

### Example 9

### Crystalline Form VIIβ 3β-tetrol

Form VIIβ was provided by allowing the melt obtained from DTA analysis of Form IIβ to solidify as the analyzed sample returned to room temperature. The solidified sample was then re-analyzed by DTA-TG with the sample uncovered. Unlike Form IIβ, the re-analyzed sample shows a single thermal transition, i.e., a prominent endotherm at about 252° C. (onset at about 239° C.) with negligible % weight loss in the thermogravimetric thermogram from between about 60° C. to about 280° C., with a temperature scan rate of 10° C./min. Thus, Form VIIβ is an anhydrate and is the more stable polymorphic form to which Form IIβ transitions prior to melting when analyzed thermally by DTA.

Form VIIβ exhibits the highest melting point of the 3β-tetrol crystalline forms disclosed herein without undergoing a polymorphic transition on heating. Therefore, Form VIIβ is expected to be stable with respect to polymorphic identity on heat or mechanical stressing and should have the most favorable shelf life if protected from moisture. This protection is required since an anhydrous form is expected to be hygroscopic, and thus Form VIIβ is expected to be unstable in comparison with the 3β-tetrol crystalline hydrates with exposure to atmospheric moisture. Thus, preference for Form VIIβ over another anhydrate of 3β-tetrol or a crystalline hydrate form is context dependent, i.e., dependent desired shelf life, formulation processing and packaging costs. In addition to the disclosed therapeutic uses, Form VIIβ is also useful for the preparation of other crystalline forms of 3β-tetrol, i.e., Form IIIβ.

### Example 10

### Crystalline Form VIIIβ 3β-tetrol

50 grams of 3β-tetrol prepared according to Example 6 was dissolved in 400 mL of anhydrous methanol, then filtered through 11 micron filter paper to remove insoluble impurities. The solution was then concentrated in vacuo (20-50 torr) to near dryness, whereupon 200 mL of ethyl acetate was added. Collection of the resulting precipitate by vacuum filtration provided crystalline material that was used in preparation of crystalline Forms IIIβ, Form IVβ, Form IXβ and Form Xβ. After drying under vacuum (<2 torr) at 100° C. for 3 hours, 42.2 g of Form VIIIβ as a white crystalline solid was obtained.

TABLE 12
Observed XRPD peaks for Form VIIIβ 3β-tetrol

| °2θ | d space (Å) | Intensity (%) |
|---|---|---|
| 6.1 ± 0.1 | 14.585 ± 0.244 | 26 |
| 7.6 ± 0.1 | 11.602 ± 0.154 | 8 |
| 8.1 ± 0.1 | 10.875 ± 0.135 | 6 |
| 9.1 ± 0.1 | 9.761 ± 0.109 | 3 |
| 9.9 ± 0.1 | 8.908 ± 0.090 | 4 |
| 11.7 ± 0.1 | 7.564 ± 0.065 | 3 |
| 12.2 ± 0.1 | 7.249 ± 0.060 | 8 |
| 13.1 ± 0.1 | 6.769 ± 0.052 | 5 |
| 13.6 ± 0.1 | 6.502 ± 0.048 | 5 |
| 14.0 ± 0.1 | 6.321 ± 0.045 | 5 |
| 14.7 ± 0.1 | 6.038 ± 0.041 | 5 |
| 15.4 ± 0.1 | 5.769 ± 0.038 | 14 |
| 16.2 ± 0.1 | 5.482 ± 0.034 | 100 |
| 18.2 ± 0.1 | 4.872 ± 0.027 | 12 |
| 18.7 ± 0.1 | 4.755 ± 0.025 | 5 |
| 19.6 ± 0.1 | 4.525 ± 0.023 | 8 |
| 19.9 ± 0.1 | 4.471 ± 0.022 | 9 |
| 20.8 ± 0.1 | 4.267 ± 0.020 | 8 |
| 21.8 ± 0.1 | 4.070 ± 0.018 | 3 |
| 23.1 ± 0.1 | 3.850 ± 0.017 | 3 |
| 24.5 ± 0.1 | 3.636 ± 0.015 | 3 |
| 25.3 ± 0.1 | 3.518 ± 0.014 | 11 |
| 27.2 ± 0.1 | 3.274 ± 0.012 | 2 |
| 28.2 ± 0.1 | 3.165 ± 0.011 | 5 |
| 29.7 ± 0.1 | 3.011 ± 0.010 | 6 |

TABLE 13
Peak listing for absorptions for Raman spectrum of Form VIIIβ

| cm⁻¹ | Intensity |
|---|---|
| 183 | 1.58 |
| 216 | 3.52 |
| 239 | 2.46 |
| 285 | 1.77 |
| 299 | 1.66 |
| 345 | 3.08 |
| 386 | 2.71 |
| 443 | 3.89 |
| 474 | 2.92 |
| 492 | 1.11 |
| 517 | 1.86 |
| 536 | 2.09 |
| 561 | 1.11 |
| 575 | 1.47 |
| 598 | 2.39 |
| 634 | 1.39 |
| 661 | 3.53 |
| 700 | 2.68 |
| 735 | 0.61 |
| 779 | 3.50 |
| 810 | 1.26 |
| 848 | 2.10 |
| 864 | 1.89 |
| 881 | 1.74 |
| 901 | 2.14 |
| 926 | 1.57 |
| 958 | 1.53 |
| 987 | 2.30 |
| 1010 | 2.51 |
| 1024 | 1.73 |
| 1057 | 2.90 |
| 1090 | 2.17 |
| 1099 | 2.22 |
| 1126 | 3.11 |
| 1147 | 1.86 |
| 1174 | 2.48 |
| 1194 | 2.56 |
| 1234 | 1.86 |
| 1244 | 1.82 |
| 1277 | 2.40 |
| 1329 | 2.93 |
| 1346 | 3.24 |
| 1373 | 1.87 |
| 1439 | 5.79 |
| 1460 | 3.23 |
| 1670 | 5.29 |
| 2657 | 0.63 |
| 2661 | 0.63 |
| 2669 | 0.61 |
| 2675 | 0.59 |
| 2688 | 0.55 |
| 2694 | 0.54 |
| 2713 | 0.60 |
| 2717 | 0.59 |
| 2727 | 0.61 |
| 2740 | 0.65 |
| 2746 | 0.65 |
| 2754 | 0.61 |
| 2777 | 0.61 |
| 2856 | 8.61 |
| 2870 | 8.58 |
| 2893 | 9.50 |
| 2902 | 9.44 |
| 2939 | 13.69 |
| 2968 | 9.07 |
| 3030 | 1.09 |
| 3263 | 0.51 |
| 3275 | 0.56 |
| 3284 | 0.60 |
| 3290 | 0.59 |
| 3302 | 0.61 |
| 3311 | 0.64 |
| 3321 | 0.65 |
| 3329 | 0.64 |
| 3344 | 0.68 |
| 3350 | 0.69 |
| 3359 | 0.68 |
| 3367 | 0.70 |
| 3373 | 0.71 |
| 3377 | 0.71 |
| 3383 | 0.71 |
| 3388 | 0.71 |
| 3396 | 0.69 |
| 3400 | 0.69 |
| 3415 | 0.65 |
| 3427 | 0.59 |

The DTA thermogram of Form VIIIβ, obtained with the sample uncovered, exhibits a broad endotherm centered at about 178° C. (onset at about 163° C.) followed by a prominent endotherm centered at about 233° C. (onset at about 225° C.). Also present is a broad endotherm centered at about 85° C.; however, this thermal transition is not associated with weight loss in the thermogravimetric thermogram where negligible % weight loss is observed from between about 60° C. to about 140° C., with a temperature scan rate of 10° C./min. Thus, the early endotherm most likely results from surface-absorbed water due to Form VIIIβ existing as a hygroscopic anhydrate.

Form VIIIβ as an anhydrate is expected to have favorable solubility compared to the crystalline hydrates and thus may show improved bioavailability. However, it is also expected to be unstable in comparison with the 3β-tetrol crystalline hydrates with exposure to atmospheric moisture and thus require protection from atmospheric moisture. Thus, preference for Form VIIIβ over another anhydrate of 3β-tetrol or a crystalline hydrate form is context dependent, i.e., dependent on route of administration, formulation processing and packaging costs. In addition to the disclosed therapeutic uses, Form VIIIβ is also useful for the preparation of other crystalline forms of 3β-tetrol, i.e., Form IIIβ, Form IVβ, Form IXβ and Form Xβ.

### Example 11

### Crystalline Form IXβ 3βtetrol

7 grams of 3β-tetrol obtained during the preparation of Form VIIIβ (immediately prior to final drying of collected solids from EtOAc precipitation) was dissolved in 20 mL of ethanol (denatured) to which 20 mL of water was added with swirling. The solution was then allowed to stand overnight at room temperature, and the resulting solids were collected by vacuum, washed with water and dried in vacuo (<2 torr) for 6 hours at 100° C. for 6 hrs to provide 5.78 g of Form IXβ.

TABLE 14
Observed XRPD peaks for Form IXβ 3β-tetrol

| °2θ | d space (Å) | Intensity (%) |
|---|---|---|
| 7.4 ± 0.1 | 11.930 ± 0.163 | 10 |
| 13.1 ± 0.1 | 6.769 ± 0.052 | 2 |
| 14.6 ± 0.1 | 6.063 ± 0.042 | 9 |
| 14.8 ± 0.1 | 5.966 ± 0.040 | 22 |
| 15.9 ± 0.1 | 5.574 ± 0.035 | 100 |
| 17.3 ± 0.1 | 5.123 ± 0.030 | 19 |
| 17.8 ± 0.1 | 4.969 ± 0.028 | 6 |
| 19.2 ± 0.1 | 4.616 ± 0.024 | 11 |
| 19.8 ± 0.1 | 4.484 ± 0.023 | 6 |
| 20.2 ± 0.1 | 4.405 ± 0.022 | 9 |
| 20.4 ± 0.1 | 4.353 ± 0.021 | 11 |
| 21.1 ± 0.1 | 4.207 ± 0.020 | 3 |
| 22.7 ± 0.1 | 3.911 ± 0.017 | 4 |
| 23.7 ± 0.1 | 3.754 ± 0.016 | 2 |
| 24.4 ± 0.1 | 3.654 ± 0.015 | 7 |
| 24.8 ± 0.1 | 3.584 ± 0.014 | 4 |
| 25.6 ± 0.1 | 3.485 ± 0.013 | 5 |
| 27.4 ± 0.1 | 3.260 ± 0.012 | 8 |
| 28.3 ± 0.1 | 3.158 ± 0.011 | 3 |
| 29.4 ± 0.1 | 3.035 ± 0.010 | 10 |

TABLE 15
Peak listing for absorptions for Raman spectrum of Form IXβ

| cm⁻¹ | Intensity |
|---|---|
| 183 | 2.16 |
| 216 | 4.63 |
| 285 | 2.26 |
| 299 | 2.22 |
| 345 | 4.02 |
| 386 | 3.78 |
| 443 | 5.29 |
| 472 | 3.75 |
| 517 | 2.49 |
| 536 | 2.91 |
| 561 | 1.49 |
| 575 | 1.83 |
| 598 | 3.19 |
| 634 | 1.78 |
| 661 | 4.20 |
| 700 | 3.50 |
| 735 | 0.78 |
| 779 | 4.16 |
| 810 | 1.61 |
| 848 | 2.44 |
| 864 | 2.25 |
| 881 | 2.02 |
| 901 | 2.67 |
| 926 | 1.93 |
| 956 | 1.87 |
| 985 | 2.67 |
| 1009 | 3.05 |
| 1024 | 2.12 |
| 1057 | 3.42 |
| 1090 | 2.53 |
| 1099 | 2.62 |
| 1126 | 3.65 |
| 1147 | 2.34 |
| 1174 | 3.11 |
| 1194 | 3.03 |
| 1221 | 2.18 |
| 1234 | 2.17 |
| 1244 | 2.30 |
| 1277 | 2.93 |
| 1304 | 2.36 |
| 1327 | 3.47 |
| 1346 | 3.75 |
| 1373 | 2.31 |
| 1379 | 2.30 |
| 1406 | 1.20 |
| 1437 | 6.80 |
| 1460 | 3.58 |
| 1670 | 6.35 |
| 2661 | 0.69 |
| 2679 | 0.64 |
| 2698 | 0.60 |
| 2704 | 0.60 |
| 2721 | 0.68 |
| 2746 | 0.71 |
| 2762 | 0.66 |
| 2835 | 5.31 |
| 2856 | 9.87 |
| 2868 | 9.72 |
| 2902 | 11.33 |
| 2931 | 16.05 |
| 2937 | 15.99 |
| 2968 | 10.04 |
| 3030 | 1.26 |
| 3286 | 0.60 |
| 3302 | 0.65 |
| 3311 | 0.68 |
| 3330 | 0.71 |
| 3340 | 0.72 |
| 3348 | 0.75 |
| 3352 | 0.75 |
| 3359 | 0.77 |
| 3369 | 0.79 |
| 3377 | 0.82 |
| 3384 | 0.84 |
| 3392 | 0.85 |
| 3402 | 0.83 |
| 3413 | 0.81 |
| 3421 | 0.77 |

The DTA thermogram of Form IXβ, obtained with the sample uncovered, exhibits a broad endotherm centered at about 180 (onset at about 165° C.) with a shoulder at about 188° C. Also present is a broad endotherm centered at about 81° C.; however, this thermal transition is not associated with weight loss in the thermogravimetric thermogram where negligible % weight loss is observed from between about 60° C. to about 140° C., with a temperature scan rate of 10° C./min. Thus, the earlier endotherm most likely results from surface-absorbed water due to Form IXβ existing as a hygroscopic anhydrate. The latter endothermic thermal transitions are associated with about 10% weight loss in the thermogravimetric thermogram and are most likely due to thermal decomposition with melting of the sample. An additional very broad endotherm may be observable between about 220° C. to 260° C. that is associated with significant weight loss in the thermogravimetric thermogram indicating further decomposition of the melt is occurring with heating.

Form IXβ as an anhydrate is expected to have favorable solubility compared to the crystalline hydrates and thus may show improved bioavailability. However, it is also expected to be unstable in comparison with the 3β-tetrol crystalline hydrates with exposure to atmospheric moisture and thus require protection from atmospheric moisture. In addition, crystalline Form IXβ has the lowest melting-decomposition points of the other anhydrates without undergoing a polymorphic transition. The weaker crystalline lattice forces in Form IXβ represented by this relatively low melting point is expected to translate to a favorable dissolution rate in water in comparison to the other anhydrates crystalline forms disclosed herein and thus Form IXβ may exhibit favorable oral bioavailability. This lower thermal stability, however is also expected to manifest itself as a shortened shelf life that may require refrigeration of formulations comprising Form IXβ. Thus, preference of Form IXβ over other anhydrates of 3β-tetrol will depend upon desired shelf life, storage costs and bioavailability considerations.

Thus, preference for Form IXβ over another anhydrate of 3β-tetrol or a crystalline hydrate form is context dependent, i.e., dependent on route of administration, formulation processing and packaging costs. In addition to the disclosed therapeutic uses, Form IXβ is also useful for the preparation of other crystalline forms of 3β-tetrol, i.e., Form VIβ.

### Example 12

### Crystalline Form Xβ 3β-tetrol

300 mg of 3β-tetrol obtained during the preparation of Form VIIIβ (immediately prior to final drying of collected solids from EtOAc precipitation) was dissolved in 3 mL of 200 proof ethanol. To this was slowly added 6 mL of ethyl acetate at room temperature. The solution was allowed to reach room temperature and allowed to stand for 60 hours. The resulting solids were collected by vacuum filtration and dried in vacuo (<2 torr) for 12 hours at room temperature to provide 226 mg of Form Xβ.

TABLE 16
Observed XRPD peaks for Form Xβ 3β-tetrol

| °2θ | Intensity (%) |
|---|---|
| 6.1 ± 0.1 | 100 |
| 8.1 ± 0.1 | 18 |
| 9.9 ± 0.1 | 10 |
| 12.1 ± 0.1 | 44 |
| 13.0 ± 0.1 | 17 |
| 13.6 ± 0.1 | 18 |
| 14.0 ± 0.1 | 19 |
| 15.8 ± 0.1 | 48 |
| 16.3 ± 0.1 | 13 |
| 16.8 ± 0.1 | 27 |
| 18.2 ± 0.1 | 86 |
| 18.6 ± 0.1 | 26 |
| 19.8 ± 0.1 | 17 |
| 20.8 ± 0.1 | 19 |
| 21.8 ± 0.1 | 23 |
| 24.3 ± 0.1 | 14 |
| 29.8 ± 0.1 | 12 |

The DTA thermogram of Form Xβ, obtained with the sample uncovered, exhibits a broad endotherm centered at about 204 (onset at about 165° C.) with a shoulder at about 217° C. Also present is a broad endotherm centered at about 81° C.; however, this thermal transition is not associated with weight loss in the thermogravimetric thermogram where negligible % weight loss is observed from between about 60° C. to about 140° C., with a temperature scan rate of 10° C./min. Thus, the earlier endotherm most likely results from surface-absorbed water due to Form Xβ existing as a hygroscopic anhydrate. The latter endothermic thermal transitions are associated with about 8% weight loss in the thermogravimetric thermogram and is most likely due to some thermal decomposition of the sample as it melts.

Form Xβ as an anhydrate is expected to have favorable solubility compared to the crystalline hydrates and thus may show improved bioavailability. However, it is also expected to be unstable in comparison with the 3β-tetrol crystalline hydrates with exposure to atmospheric moisture and thus require protection from atmospheric moisture. In addition, crystalline Form Xβ has the one of the lower melting-decomposition points of the anhydrates without undergoing a polymorphic transition. The weaker crystalline lattice forces in Form Xβ represented by this relatively low melting point is expected to translate to a favorable dissolution rate in water and thus Form Xβ may exhibit favorable oral bioavailability. This lower thermal stability, however, may manifest itself as a shortened shelf life, but is not expected to be as problematic in this regard as Form IXβ, which has the lowest melting-decomposition point. Thus, Form Xβ is expected to exhibit intermediate behavior with lower expected bioavailability compensated by an improved shelf life. Thus, preference of Form Xβ over other anhydrates of 3β-tetrol will depend upon desired shelf life, storage costs and bioavailability considerations.

### Example 13

### Treatment of Inflammation—Metabolic Conditions

Glucose lowering in 8 week old db/db diabetic mice: The hyperinsulinemic-euglycemic clamp protocol was conducted to measure insulin sensitivity in vivo. In this procedure, insulin was administered to raise the insulin concentration while glucose was infused to maintain euglycemia or a fixed, normal blood glucose level (about 180 mg/dL). The glucose infusion rate (GIR) needed to maintain euglycemia showed insulin action in these animals. The objective of this protocol was to investigate characterize the capacity of 17α-ethynylandrost-5-ene-3β,7β,17β-triol and androst-5-ene-3β,7β,16α,17β-tetrol to ameliorate systemic insulin resistance and improve whole body glucose disposal in the hyperinsulinemic-euglycemic clamp model. The degree of skeletal muscle and hepatic insulin sensitivity and tissue specific glucose uptake were also assessed. The animals were dosed daily by oral gavage for 14 days. On Day 10 of treatment catheters were implanted in the carotid artery and jugular vein. On the day of the clamp (day 14) the compound was administered at 7:30 am.

Body weight and glucose concentration were assessed on day 0, 7 and day 14 of treatment. On day 14 a euglycemic-hyperinsulinemic clamp was performed. Food was removed at 7:30 am and at 10:30 a primed continuous infusion of [3-³H]-glucose (0.05 μCi/min) was administered. A baseline blood sample was taken at 12:50 (−10 min) and at 1:00 (0 min) a euglycemic-hyperinsulinemic clamp was initiated by administering 10 mU/kg/min of insulin. Glucose was infused at a variable rate to clamp the glucose concentration at about 180 mg/dl. A bolus of [¹⁴C]-2deoxyglucose was given at the end of the study to assess tissue specific glucose uptake. Plasma ¹⁴C 2-deoxyglucose was assessed at 122, 125, 130, 135, 145 min. The animals were then anesthetized with an intravenous infusion of sodium pentobarbital and selected tissues were removed, immediately frozen in liquid nitrogen and stored at −70° C. until analysis.

Analysis was conducted as follows. Plasma samples were deproteinized with Ba(OH)₂ (0.3 N) and ZnSO₄ (0.3 N), dried and radioactivity was assessed on scintillation counter (Packard TRICARB 2900 TR, Meriden, Conn.). Frozen tissue samples were homogenized in 0.5% perchloric acid, centrifuged and neutralized. One supernatant was directly counted to determine radioactivity from both [₁₄C] DG and DGP. A second aliquot was treated with Ba(OH)₂ and ZnSO₄ to remove ¹⁴C DGP and any tracer incorporated into glycogen and then counted to determine radioactivity from free [¹⁴C]DG(2). [¹⁴C]DGP was calculated as the difference between the two aliquots. The accumulation of [¹⁴C]DGP was normalized to tissue weight and tracer bolus. Rg, an index of tissue specific glucose uptake was calculated as previously described (E. W. Kraegen et al., Am. J. Physiol., 248: E353-E362 (1985)). Whole body glucose turnover was calculated as the ratio of the ³H glucose infusion rate (dpm/kg/min) and arterial plasma glucose specific activity (dpm/mg). Endogenous glucose production was calculated as the difference between the whole body glucose turnover and the exogenous glucose infusion rate (R. N. Bergman et al., Endocr. Rev., 6:45-86, (1985)). Treatment groups are summarized in the table shown below.

| | | Dosing volume and | |
|---|---|---|---|
| | | dosing solution | |
| Group | Treatment | concentration | N |
| A—vehicle control* | vehicle 8 mL/kg, po, | 8 mL/kg | 10 |
| | bid for 13 days, | | |
| | qd on day 14 | | |
| B—compound 1** | 40 mg/kg, po, | 4 mL/kg of 10 mg/mL | 10 |
| | bid for 13 days, | stock in vehicle | |
| | qd on day 14 | | |
| C—compound 1** | 80 mg/kg, po, | 8 ml/kg of 10 mg/ml | 10 |
| | bid for 13 days, | stock in vehicle | |
| | qd on day 14 | | |
| D—compound 2** | 40 mg/kg, po, | 4 mL/kg of 10 mg/mL | 10 |
| (3β-tetrol) | bid for 13 days, | in vehicle | |
| | qd on day 14 | | |
| E—positive*** | 25 mg/kg, po, | 5 mL/kg of 5 mg/mL | 10 |
| control | bid for 13 days, | in water + 1% CMC | |
| | qd on day 14 | | |

*vehicle: 30% sulfobutylether in water (20 mg/mL of drug in solution for groups B-D)
**compound 1: 17α-ethynylandrost-5-ene-3β,7β,17β-triol compound 2: androst-5-ene-3β,7β,16α,17β-tetrol
***rosiglitazone maleate (31493r, AApin Chemicals Limited (UK), CMC—Carboxymethyl cellulose (medium grade, C4888, Sigma)

The insulin dose was 10 mU/kg/min. In a normal animal, this dose of insulin would require infusion of about 90 mg/kg/min of glucose to keep the glucose level clamped at about 150 mg/dl. The average glucose requirement in all treatment groups was about 50% of normal. The results showed that both 17α-ethynylandrost-5-ene-3β,7β,17β-triol and androst-5-ene-3β,7β,16α,17β-tetrol (3β-tetrol) increased the glucose infusion rate compared to the vehicle control, which means insulin action was improved in the groups B, C, D and E.

Using the 3-³H glucose tracer, the rate of liver glucose production was calculated during the basal period and the ability of insulin to suppress liver glucose production during the clamp. In severe insulin resistant animals endogenous glucose production would decrease by about 50% with the insulin dose that was used. In groups C, D and E, insulin completely suppressed endogenous glucose production (p<0.05), which showed an improvement in hepatic insulin action.

To assess peripheral insulin action, tissue specific glucose uptake during the euglycemic-hyperinsulinemic clamp was assessed using ¹⁴C-2-deoxyglucose. A bolus of ¹⁴C-2-deoxyglucose was given at 120 min. Tissues were collected 25 minutes later. Tissues were analyzed for total accumulation of ¹⁴C-2-deoxyglucose phosphate. In this protocol, brain glucose uptake is unaffected by most treatment regimens and it thus serves as an internal control. The results showed that brain glucose uptake was comparable between all of the groups. In the heart and diaphragm, glucose uptake was higher in the treated groups compared to the vehicle control group. Both androst-5-ene-3β,7β,16α,17β-tetrol (3β-tetrol) and rosiglitazone were more effective (p<0.05) in augmenting muscle glucose uptake in the gastrocnemius muscle. In white vastus muscle, which is a non oxidative muscle group, differences were not detected except between androst-5-ene-3β,7β,16α,17β-tetrol and rosiglitazone.

### Example 14

### Treatment of Inflammation—Bowel Inflammation Conditions

The capacity of androst-5-ene-3β,7β,16α,17β-tetrol (3β-tetrol) to limit or inhibit inflammation or symptoms of inflammation is shown using an animal model for inflammatory bowel disease using the following protocol.

Groups of 3 male Wistar rats (180±20 grams) fasted for 24 hours before 2,4-dinitrobenzene sulfonic acid (DNBS) or saline challenge are used. Distal colitis is induced by intra-colonic instillation of 0.5 mL of an ethanolic solution of DNBS (30 mg in 0.5 mL of a 30% ethanol in saline solution) after which 2 mL of air was injected through the cannula to ensure that the solution remained in the colon. The volume used was 0.1 mL per injection of 2 and 20 mg/mL of compound such as androst-5-ene-3β,7β,17β-triol or 3β-tetrol in a liquid formulation, which was administered by subcutaneous injection once a day for 6 days (0.2 mg/animal/day or 2.0 mg/animal/day). The formulation contains 100 mg/mL of compound. Concentrations of 2 mg/mL and 20 mg/mL are obtained by diluting the 20 mg/mL formulation with vehicle that lacked compound.

The first dose is given 30 minutes after DNBS challenge. Sulfasalazine (30 mg/mL in 2% Tween 80 in distilled water) was administered orally (PO) once a day (10 mL/kg/day) for 7 days, the first two doses beginning 24 hours and 2 hours before DNBS challenge. The presence of diarrhea is recorded daily by examining the anal area. Animals are fasted for 24 hours prior to being sacrificed. Animals are sacrificed on day 7 or day 8 and their colons are removed and weighed. Before removal of the colon, signs of adhesion between the colon and other organs are recorded. Also, the presence of ulcerations is noted after weighing of each colon. The “net” change of colon-to-body weight (BW) ratio is normalized relative to saline-challenged baseline group. A 25-30% decrease in “net” colon-to-body weight ratio is considered significant. The results showed that androst-5-ene-3β,7β,17β-triol had a modest effect on the course of disease (about 15-20% decrease in net colon-to-body weight ratio), while treatments with androst-5-ene-3β,7β,16α,17β-tetrol is effective (about 25-35% decrease in net colon-to-body weight ratio).

Variations of this protocol include administration of compounds in an aqueous solution with or without 30% sulfobutylether-cyclodextrin in water using dose levels described above and/or one or more of 0.05 mg/animal/day, 0.1 mg/animal/day, 0.5 mg/animal/day and 1.0 mg/animal/day.

### Example 15

The capacity of 5-androstene-3β,7β,17β-triol, androst-5-ene-3β,7β,16α,17β-tetrol (3β-tetrol) and other compounds to reverse adverse effects of glucocorticoids in bone growth was shown in the human MG-63 osteosarcoma cell line. MG-63 cells are osteoblasts, which are cells that mediate bone growth. This cell line has been used extensively to study bone biology and to characterize the biological activity of compounds for treatment of bone loss conditions (e.g., B. D. Boyan et al., J. Biol. Chem., 264(20):11879-11886 (1989); L. C. Hofbauer et al., Endocrinology, 140(10):4382-4389 (1999)). Adverse toxicities associated with elevated glucocorticoid levels include a decrease in the production of IL-6 and IL-8 by osteoblasts, including the MG-63 cell line, and an increase in the expression of the 11β-hydroxysteroid dehydrogenase type 1 enzyme (11β-HSD). Increased 11β-hydroxysteroid dehydrogenase type 1 enzyme results in increased levels of endogenous glucocorticoid activity by converting endogenous cortisone to the active cortisol, which inhibits bone growth. The 11β-HSD enzyme is expressed in liver, adipose tissue, brain and bone tissues. Cortisol generated by 11β-HSD-1 contributes to osteoporosis, insulin resistance, type 2 diabetes, dyslipidemia, obesity, central nervous system disorders such as stroke, neuron death, depression and Parkinson Disease. Decreases in IL-6, IL-8 and osteoprotegerin are associated with decreased bone growth by osteoblasts. Pilot studies showed that the IC₅₀ for inhibition of IL-6 from MG-63 cells by dexamethasone was 10 nM and the IC₅₀ for inhibition of growth of MG-63 cells by dexamethasone was 15.3 nM. In this protocol, MG-63 cells are grown in the presence or absence of the synthetic glucocorticoid dexamethasone at a 30 nM concentration and in the presence or absence of test compound.

These results showed that the test compounds at 10 nM partially reversed the adverse effects of dexamethasone at 30 nM, which shows that the compounds can reverse multiple toxicities associated with elevated glucocorticoid levels in osteoblasts, which are the cells that mediate bone growth. Osteoprotegerin is a factor associated with bone growth and decreased osteoprotegerin synthesis is associated with bone loss. Compound 1B (3β-tetrol) completely or partially reversed the decrease in osteoprotegerin synthesis by MG-63 cells in the presence of 30 nM dexamethasone (normal osteoprotegerin levels at 0.1 μM).

To show that relevant effects could be obtained in vivo, 3β-tetrol is administered to mice that were also treated daily with dexamethasone for 23 days to reduce levels of osteoprotegerin in the animals. Osteoprotegerin levels in mice that are treated with vehicle and dexamethasone at 10 μg/day (positive control group) typically show 3.3 pMol/L osteoprotegerin,

The degree of apoptosis of osteoblasts and osteocytes in murine vertebral bone as a function of estrogen deficiency was examined. Swiss Webster mice (four months old) were ovariectomized. Twenty-eight days later, the animals were sacrificed, vertebrae were isolated, fixed and embedded, and then un-decalcified in methacrylate. The prevalence of osteoblast and osteocyte apoptosis was determined by the TUNEL method with CuSO₄ enhancement, and was found to be increased following loss of estrogen.

Collectively, the results described in this example are evidence that compounds such as 3β-tetrol affect bone tissue by both increasing bone growth and by inhibiting bone loss. In addition, 3β-tetrol does not interact with androgen receptor, estrogen receptor-α or estrogen receptor-β, which is consistent with their capacity to treat bone loss conditions without exerting unwanted sex hormone activity.

What is claimed is:

1. Crystalline androst-5-ene-3β,7β,16α,17β-tetrol provided that crystalline androst-5-ene-3β,7β,16α,17β-tetrol is not Form Iβ 3β-tetrol.

2. The crystalline androst-5-ene-3β,7β,16α,17β-tetrol of claim 1 wherein crystalline androst-5-ene-3β,7β,16α,17β-tetrol is Form IIβ, Form IIIβ, Form IVβ, Form Vβ, Form VIβ, Form VIIβ, Form VIIIβ, Form IXβ or Form Xβ 3βtetrol.

3. The crystalline androst-5-ene-3β,7β,16α,17β-tetrol of claim 1 wherein crystalline androst-5-ene-3β,7β,16α,17β-tetrol is Form IIβ 3β-tetrol characterized by DTA thermogram, obtained with a temperature ramp of 10° C./min, having a prominent endotherm centered at about 252° C., an endotherm centered at 239° C. with onset temperature of about 235° C. and an exotherm centered at about 242° C.

4. The crystalline androst-5-ene-3β,7β,16α,17β-tetrol of claim 3 wherein crystalline androst-5-ene-3β,7β,16α,17β-tetrol is further characterized a TGA thermogram, obtained with a temperature ramp of 10° C./min, having 5% wt loss from between about 60° C. to about 140° C. associated with a broad endotherm in the DTA thermogram centered at about 102° C.

5. The crystalline androst-5-ene-3β,7β,16α,17β-tetrol of claim 1 wherein crystalline androst-5-ene-3β,7β,16α,17β-tetrol is Form IIIβ 3β-tetrol characterized by (1) XRPD pattern having three or more peaks selected from the group consisting of about 7.6, 14.9, 25.4 and 29.6 degree 2-theta and one or more peaks selected from the group consisting of about 15.4, 16.1, 17.3 and 19.9 degree 2-theta or (2) solid state Raman spectrum with absorbances at about 1275, 1329, 1344 and 1437 cm⁻¹ and one or more absorbances selected from the group consisting of about 445, 474, 987, 1057, 1091 and 1128 cm⁻¹ or (1) and (2).

6. The crystalline androst-5-ene-3β,7β,16α,17β-tetrol of claim 5 wherein Form IIIβ 3β-tetrol is further characterized by (1) DTA thermogram, obtained with a temperature ramp of 10° C./min, having a prominent endotherm centered at about 200° C. having an onset temperature of about 191° C. and a shoulder at about 210° C. or (2) TGA thermogram, obtained with a temperature ramp of 10° C./min, having about 5% wt loss from between about 60° C. to about 140° C. associated with a broad endotherm in the DTA thermogram centered at about 103° C. and between about 5 to about 10% wt loss or more associated with the DTA 200° C. endotherm or (1) and (2).

7. The crystalline androst-5-ene-3β,7β,16α,17β-tetrol of claim 1 wherein crystalline androst-5-ene-3β,7β,16α,17β-tetrol is Form IVβ 3β-tetrol characterized by (1) XRPD pattern having three or more peaks selected from the group consisting of about 7.7, 15.4, 16.2 and 25.3 degree 2-theta and one or more peaks selected from the group consisting of about 14.8, 19.7, 20.8 and 29.9 degree 2-theta or (2) solid state Raman spectrum with absorbances at about 1279, 1329, 1342 and 1437 cm⁻¹ and one or more absorbances selected from the group consisting of about 443, 474, 517, 536, 901, 985, 1009, 1045, 1090, 1099 and 1172 cm⁻¹ or (1) and (2).

8. The crystalline androst-5-ene-3β,7β,16α,17β-tetrol of claim 7 wherein crystalline androst-5-ene-3β,7β,16α,17β-tetrol is further characterized by (1) DTA thermogram, obtained with a temperature ramp of 10° C./min, having a prominent endotherm centered at about 233° C. with an onset temperature of about 228° C. and a weak, broad endotherm centered at about 197° C. with an onset temperature of about 189° C. or (2) TGA thermogram, obtained with a temperature ramp of 10° C./min, having about 5% wt loss from between about 60° C. to about 140° C. associated with a broad endotherm in the DTA thermogram centered at about 99° C. or (1) and (2).

9. The crystalline androst-5-ene-3β,7β,16α,17β-tetrol of claim 1 wherein crystalline androst-5-ene-3β,7β,16α,17β-tetrol is Form Vβ 3β-tetrol characterized by (1) XRPD pattern having three or more peaks selected from the group consisting of about 7.4, 14.8, 15.9, 17.3, 19.2, 20.2, 24.4 and 29.4 degree 2-theta and one or more peaks selected from the group consisting of about 14.6, 17.8, 19.8, 20.3, 21.1, 22.7, 25.5 and 27.3 degree 2-theta or (2) solid state Raman spectrum with absorbances at about 1279, 1329, 1344 and 1439 cm⁻¹ and one or more absorbances selected from the group consisting of about 443, 472, 536, 985, 1009, 1055, 1099 and 1172 cm⁻¹ or (1) and (2).

10. The crystalline androst-5-ene-3β,7β,16α,17β-tetrol of claim 9 wherein crystalline androst-5-ene-3β,7β,16α,17β-tetrol is further characterized by (1) DTA thermogram, obtained with a temperature ramp of 10° C./min, having a prominent endotherm centered at about 230° C. with an onset temperature of about 223° C. and a weak exotherm at about 188° C. or (2) TGA thermogram, obtained with a temperature ramp of 10° C./min, having about 5% wt loss from between about 60° C. to about 140° C. associated with a broad endotherm in the DTA thermogram centered at about 102° C. or (1) and (2).

11. The crystalline androst-5-ene-3β,7β,16α,17β-tetrol of claim 1 wherein crystalline androst-5-ene-3β,7β,16α,17β-tetrol is Form VIβ 3β-tetrol characterized by (1) XRPD pattern having three or more peaks selected from the group consisting of about 6.5, 7.7, 8.2, 13.1, 15.0, 15.4, 16.2, 17.0, 19.9, 22.3 and 25.4 degree 2-theta and one or more peaks selected from the group consisting of about 9.7, 14.8, 15.9, 16.7, 19.3, 19.6, 20.8, 20.9, 21.1, 21.2, 21.9, 23.1, 23.5, 23.9, 24.6, 25.3 and 29.8 degree 2-theta or (2) solid state Raman spectrum with four or more absorbances selected from the group consisting of about 1196, 1232, 1250, 1273, 1319, 1344, 1439 and 1462 cm⁻¹ and one or more absorbances selected from the group consisting of about 440, 447, 476, 519, 696, 901, 987, 1007, 1036, 1059 and 1091 cm⁻¹ or (1) and (2).

12. The crystalline androst-5-ene-3β,7β,16α,17β-tetrol of claim 11 wherein crystalline androst-5-ene-3β,7β,16α,17β-tetrol is further characterized by (1) DTA thermogram, obtained with a temperature ramp of 10° C./min, having a prominent endotherm centered at about 233° C. with an onset temperature of about 226° C. and no endotherm between about 140° C. to about 200° C. or (2) TGA thermogram, obtained with a temperature ramp of 10° C./min, having about 10% wt loss from between about 60° C. to about 140° C. associated with a broad endotherm in the DTA thermogram centered at about 111° C. or (1) and (2).

13. The crystalline androst-5-ene-3β,7β,16α,17β-tetrol of claim 1 wherein crystalline androst-5-ene-3β,7β,16α,17β-tetrol is Form VIIβ 3β-tetrol characterized by DTA thermogram, obtained with a temperature ramp of 10° C./min, having a prominent endotherm centered at about 252° C. with an onset temperature of about 239° C., and no thermal transitions from between about 60° C. to about the onset temperature of the DTA 252° C. endotherm.

14. The crystalline androst-5-ene-3β,7β,16α,17β-tetrol of claim 1 wherein crystalline androst-5-ene-3β,7β,16α,17β-tetrol is Form VIIIβ 3β-tetrol characterized by (1) XRPD pattern having three or more peaks selected from the group consisting of about 6.1, 12.2, 16.2 and 25.3 degree 2-theta and one or more peaks selected from the group consisting of about 7.6, 8.1, 15.4, 18.2, 19.6, 19.9, 20.8 and 29.8 degree 2-theta or (2) solid state Raman spectrum with absorbances at about 1277, 1329, 1346 and 1439 cm⁻¹ and one four or more absorbances selected from the group consisting of about 443, 474, 987, 1010, 1057 and 1099 cm⁻¹ or (1) and (2).

15. The crystalline androst-5-ene-3β,7β,16α,17β-tetrol of claim 14 wherein crystalline androst-5-ene-3β,7β,16α,17β-tetrol is further characterized by (1) DTA thermogram, obtained with a temperature ramp of 10° C./min, having a prominent endotherm centered at about 233° C. with an onset temperature of about 239° C. and a broad endotherm centered at about 178° C. with an onset temperature of about 163° C. or (2) TGA thermogram, obtained with a temperature ramp of 10° C./min, having negligible % wt loss from between about 60° C. to about 140° C. associated with a broad endotherm in the DTA thermogram centered at about 85° C. of variable intensity or (1) and (2).

16. The crystalline androst-5-ene-3β,7β,16α,17β-tetrol of claim 1 wherein crystalline androst-5-ene-3β,7β,16α,17β-tetrol is Form IXβ 3β-tetrol characterized by (1) XRPD pattern having three or more peaks selected from the group consisting of about 7.4, 14.9, 15.9, 17.3, 20.4 and 24.4 degree 2-theta and one or more peaks selected from the group consisting of about 14.6, 17.9, 19.2, 19.8, 20.2, 25.6, 27.4 and 29.4 degree 2-theta or (2) solid state Raman spectrum with absorbances at about 1277, 1329, 1346 and 1439 cm⁻¹ and one or more absorbances selected from the group consisting of about 443, 472, 536, 598, 901, 985, 1009, 1057 and 1099 cm⁻¹.

17. The crystalline androst-5-ene-3β,7β,16α,17β-tetrol of claim 16 wherein crystalline androst-5-ene-3β,7β,16α,17β-tetrol is further characterized by (1) DTA thermogram, obtained with a temperature ramp of 10° C./min, having a prominent endotherm centered at about 180° C. with an onset temperature of about 165° C. and a shoulder at about 187° C. or (2) TGA thermogram, obtained with a temperature ramp of 10° C./min, having about 5 to about 10% wt loss or more associated with the DTA 180° C. endotherm and about 5 to about 10% wt loss or more associated with a very broad endotherm in the DTA thermogram between about 220° C. to about 260° C. or (1) and (2).

18. The crystalline androst-5-ene-3β,7β,16α,17β-tetrol of claim 1 wherein crystalline androst-5-ene-3β,7β,16α,17β-tetrol is Form Xβ 3βtetrol characterized by (1) XRPD pattern having three or more XRPD peaks selected from the group consisting of about 6.1, 12.1, 13.0, 13.6, 14.0, 15.8, 18.2 and 18.6 degree 2-theta and one or more peaks selected from the group consisting of about 8.1, 9.9, 16.8, 19.8, 20.8, 21.8, 24.3 and 29.8 degree 2-theta.

19. The crystalline androst-5-ene-3β,7β,16α,17β-tetrol of claim 18 wherein crystalline androst-5-ene-3β,7β,16α,17β-tetrol is further characterized by (1) DTA thermogram, obtained with a temperature ramp of 10° C./min, having a prominent endotherm centered at about 204° C. with an onset temperature of about 190° C. or a shoulder at 217° C. or (2) TGA thermogram, obtained with a temperature ramp of 10° C./min, having negligible % wt loss from between about 60° C. to about 140° C. associated with a broad endotherm in the DTA thermogram centered at about 81° C. of variable intensity and about 5 to about 10% wt loss or more associated with the DTA 204° C. endotherm or (1) and (2).

20. A crystalline hydrate of androst-5-ene-3β,7β,16α,17β-tetrol provided that the crystalline hydrate is not Form Iβ 3β-tetrol.

21. The crystalline hydrate of androst-5-ene-3β,7β,16α,17β-tetrol wherein the crystalline hydrate is a monohydrate or a dihydrate.

22. The crystalline hydrate of claim 20 wherein the hydrate is Form IIβ, Form IIIβ, Form IVβ, Form Vβ or Form VIβ 3β-tetrol.

23. A crystalline anhydrate of androst-5-ene-3β,7β,16α,17β-tetrol.

24. The crystalline anhydrate of claim 23 where the anhydrate is Form VIIβ, Form VIIIβ, Form IXβ or Form Xβ 3βtetrol.

25. A formulation comprising or consisting essentially of one or more pharmaceutically acceptable excipients and a solid state form of androst-5-ene-3β,7β,16α,17β-tetrol provided the solid state form is not Form Iβ 3β-tetrol.

26. The formulation of claim 25 wherein the formulation is an oral, parenteral, buccal, sublingual or topical formulation.

27. The formulation of claim 25 wherein the solid state form is crystalline androst-5-ene-3β,7β,16α,17β-tetrol.

28. The formulation of claim 25 wherein the solid state form is a crystalline hydrate of androst-5-ene-3β,7β,16α,17β-tetrol.

29. The formulation of claim 28 wherein the crystalline hydrate is a monohydrate or a dihydrate.

30. The formulation of claim 28 wherein the crystalline hydrate is Form IIβ, Form IIIβForm IVβ, Form Vβ or Form VIβ 3β-tetrol.

31. The formulation of claim 25 wherein the solid state form is a crystalline anhydrate of androst-5-ene-3β,7β,16α,17β-tetrol.

32. The formulation of claim 31 wherein the crystalline anhydrate is Form VIIβ, Form VIIIβ, Form IXβ or Form Xβ 3β-tetrol.

33. A method of preparing a liquid or suspension formulation comprising admixing a solid state form of androst-5-ene-3β,7β,16α,17β-tetrol with a pharmaceutically acceptable liquid excipient provided the solid state form is not Form Iβ 3β-tetrol.

34. The method of claim 33 wherein the solid state form is crystalline androst-5-ene-3β,7β,16α,17β-tetrol.

35. The method of claim 33 wherein the solid state form is a crystalline hydrate of androst-5-ene-3β,7β,16α,17β-tetrol.

36. The method of claim 35 wherein the crystalline hydrate is a monohydrate or a dihydrate.

37. The method of claim 35 wherein the crystalline hydrate is Form IIβ, Form IIIβ, Form IVβ, Form Vβ or Form VIβ 3β-tetrol.

38. The method of claim 33 wherein the solid state form is a crystalline anhydrate of androst-5-ene-3β,7β,16α,17β-tetrol.

39. The method of claim 38 wherein the crystalline anhydrate is Form VIIβ, Form VIIIβ, Form IXβ or Form Xβ 3βtetrol.

40. A method of treating unwanted inflammation, comprising administering an effective amount of a solid formulation to a subject in need thereof wherein the solid formulation comprises or consists essentially of a solid state form of androst-5-ene-3β,7β,16α,17β-tetrol, provided the solid state form is not Form Iβ 3β-tetrol, and one or more pharmaceutically acceptable excipients.

41. The method of claim 40 wherein the solid state form is crystalline androst-5-ene-3β,7β,16α,17β-tetrol.

42. The method of claim 40 wherein the solid state form is a crystalline hydrate of androst-5-ene-3β,7β,16α,17β-tetrol.

43. The method of claim 42 wherein the crystalline hydrate is a monohydrate or a dihydrate.

44. The method of claim 42 wherein the crystalline hydrate is Form IIβ, Form IIIβ, Form IVβ, Form Vβ or Form VIβ 3β-tetrol.

45. The method of claim 40 wherein the solid state form is a crystalline anhydrate of androst-5-ene-3β,7β,16α,17β-tetrol.

46. The method of claim 45 wherein the crystalline anhydrate is Form VIIβ, Form VIIIβ, Form IXβ or Form Xβ 3β-tetrol.

47. The method of claim 40 wherein the unwanted inflammation is a condition or disease associated with chronic, non-production inflammation.

48. The method of claim 40 wherein the condition or disease is an autoimmune condition or disease.

49. The method of claim 40 wherein the condition or disease is a metabolic condition or disease.

50. The method of claim 49 wherein the metabolic condition or disease is type 2 diabetes, obesity, insulin resistance, hyperglycemia, impaired glucose utilization or tolerance, or impaired or reduced insulin synthesis.
